(19) United States
(12) Patent Application Publication
(10) Pub. No.: US 2023/0111145 A1
(43) Pub. Date: Apr. 13, 2023

(54) METHODS FOR DETECTING PROTEINS ASSOCIATED WITH AD

(71) Applicant: Carlos Cruchaga, St. Louis, MO (US)

(72) Inventor: Carlos Cruchaga, St. Louis, MO (US)

(73) Assignee: Washington University, St. Louis, MO (US)

(21) Appl. No.: 17/964,714

(22) Filed: Oct. 12, 2022

Related U.S. Application Data
(60) Provisional application No. 63/254,732, filed on Oct. 12, 2021.

(51) Int. Cl. G01N 33/68

(52) U.S. Cl. CPC G01N 33/6896; G01N 2800/2821; G01N 2800/52

(57) ABSTRACT

Among the various aspects of the present disclosure is the provision of detecting proteins associated with Alzheimer's disease (AD) or risk variant thereof; diagnosis, prognosis, and monitoring of disease progression; or monitoring treatment.

### CROSS-REFERENCE TO RELATED APPLICATIONS

This application claims priority from U.S. Provisional Application Ser. No. 63/254,732 filed on 12 Oct. 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to detecting proteins associated with Alzheimer's disease (AD) or risk variant thereof; diagnosis, prognosis, and monitoring of disease progression; or monitoring treatment.

### SUMMARY OF THE INVENTION

Among the various aspects of the present disclosure is the provision of detecting proteins associated with Alzheimer's disease (AD) or risk variant thereof; diagnosis, prognosis, and monitoring of disease progression; or monitoring treatment.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 shows an exemplary embodiment of a study outline in accordance with the present disclosure. In discovery stage, protein measures with SOMAscan targeting 1,305 proteins were obtained in brain, CSF, and plasma tissues from well-characterized Knight ADRC and DIAN participants comprehensive clinical information about AD pathology and cognition. This discovery cohort contained sporadic AD (290 in brain; 176 in CSF; 105 in plasma), TREM2 risk variant carriers (21 in brain; 47 in CSF; 131 in plasma), autosomal dominant AD (24 in brain), and healthy controls (25 in brain; 494 in CSF; 254 in plasma). Using this large number of samples, differential abundance analyses were performed for sporadic AD status, TREM2 risk variant status and autosomal dominant AD status. Several publicly available external proteomics data were then used to replicate findings. In addition, quantitative analyses using several quantitative neuropathology measures, including CDR and Braak scores in brain and age at onset in three tissues, were performed. Finally, replication proteins were used for creating a tissue-specific prediction models and pathway enrichment analysis.

FIG. 2(A-B) shows an exemplary embodiment of multi-tissue proteomics profiling of sporadic AD in accordance with the present disclosure. FIG. 2A shows volcano plots for brain, CSF, and plasma tissue. Multiple proteins (12 in brain; 117 in CSF; 26 in plasma; shown in black) are differentially abundant in AD (compared to healthy controls) at the Bonferroni adjusted significance. A subset of those identified proteins also showed differential abundance levels in the other tissue. FIG. 2B shows tissue-specific prediction models for discovery data and externally replicated data set. For example, in CSF, among 117 proteins showing differential abundance levels in AD, 39 proteins (including SMOC1, Calcineurin, and ERK-1) were externally replicated showing nominal significance (P<0.05) and same direction of effects. The prediction model using these 39 proteins provided an AUC of 0.89 in the Knight ADRC data and 0.9 in the Emory-ADRC data. In addition, the 12 proteins selected based on stepwise discriminant analysis (14-3-3 protein zeta/delta, EphA5, Calcineurin, Somatostatin-28, Cyclophilin A, Contactin-5, GFAP, Corticotropin-lipotropin, Spondin-1, TCTP, PolyUbiquitin K48, and Peroxiredoxin-6, Supplementary Table 14 in FIG. 19) led to an AUC of 0.88 in discovery and 0.999 in replication data. This showed that this predication model outperformed the gold standard CSF Aβ/tau181 ratio (with P=2.4×10-6).

FIG. 3(A-B) shows an exemplary embodiment of multi-tissue proteomics profiling of TREM2 variant carrier status in accordance with the present disclosure. FIG. 3A shows volcano plots for brain, CSF, and plasma tissue. Multiple proteins showed differential abundance levels in TREM2 variant carriers (compared to controls or other sporadic AD cases) in at least one of the three tissues. A subset of those identified proteins were replicated in the other tissues. For example, among 38 proteins associated with the TREM2 variant carrier status, 7 proteins (Supplementary Table 14 in FIG. 19) were replicated in brain and plasma. FIG. 3B shows a prediction model based on the across-tissue replicated proteins (the upper protein curve (present for CSF and Plasma models) with proteins validated by the other two tissues and the lower protein curve (present for Plasma models only) based on the subset chosen through the discriminant analysis) showed higher accuracy than the well-accepted p-Tau/Aβ42 ratio (shown in black), while including age and sex as covariates. In plasma, out of 26 proteins, the 9 proteins selected based on stepwise discriminant analysis are Bone proteoglycan II, STAT3, uPA, ERK-1, VCAM-1, PAPP-A, BSSP4, XTP3A, and S100A4. The prediction models of identified proteins while including age, sex and APOE status as covariates provided similar performance.

FIG. 4(A-B) shows an exemplary embodiment of proteomic profiling of autosomal dominant AD (ADAD) abundance in accordance with the present disclosure. FIG. 4A shows 109 proteins associated with the ADAD mutation carrier status at Bonferroni corrected threshold (volcano plot described above) and the 17 proteins (Supplementary Table 12 in FIG. 17; Supplementary Table 14 in FIG. 19) were replicated in CSF and in the same direction. The model with these 17 proteins provided significantly higher AUC than the age alone (AUC=1 vs 0.76; P=9.9×10⁻³ in brain and AUC=0.87 vs 0.53, P<2.2×10⁻¹⁶ in CSF). FIG. 4B shows 12 proteins associated with sporadic AD brains displayed even stronger effect size in the ADAD mutation carrier brains. The effect of ADAD status on log-transformed protein levels (y-axis) roughly corresponded to 1.4 times the effect of AD status (x-axis) among the 12 identified proteins. This slope estimate (1.39, standard error=0.21, P=3.8×10⁻⁵) was obtained by fitting a regression line going through the origin, which explained the scatterplot better than a regression line allowing the intercept (Multiple R-squared value=0.80 without intercept vs. 0.65 with non-zero intercept). The box plots for the select 5 proteins are displayed.

FIG. 5(A-B) shows an exemplary embodiment of pathway enrichments for multi-tissue findings in accordance with the present disclosure. The dot chart (FIG. 5A)(size corresponding to the number of identified genes and shade corresponding to the FDR corrected significance) presents that several identified proteins (FIG. 5B)(Calcinuerin, APOE and α-synuclein) enrich in several pathways including Alzheimer's disease, Parkinson's disease and several immune related pathways (Supplementary Table 15 in FIG. 20A (20A-1 through 20A-9), 20B (20B-1 through 20B-9), and 20C (20C-1 through 20C-6)).

FIG. 6(A-D) shows Supplementary Table 1 in accordance with the present disclosure. FIG. 6A, FIG. 6B, FIG. 6C, and FIG. 6D are consecutive sheets of the table.

FIG. 7(A-C) shows Supplementary Table 2 in accordance with the present disclosure. FIG. 7A, FIG. 7B, and FIG. 7C are consecutive sheets of the table.

FIG. 8(A-C) shows Supplementary Table 3 in accordance with the present disclosure. FIG. 8A (8A-1 through 8A-4) shows the first section of Supplementary Table 3. FIG. 8B (8B-1 through 8B-4) shows the second section of Supplementary Table 3. FIG. 8C shows the third section of Supplementary Table 3.

FIG. 9(A-B) shows Supplementary Table 4 in accordance with the present disclosure. FIG. 9A and FIG. 9B are consecutive sheets of the table.

FIG. 10 shows Supplementary Table 5 in accordance with the present disclosure.

FIG. 11(A-B) shows Supplementary Table 6 in accordance with the present disclosure. FIG. 11A and FIG. 11B are consecutive sheets of the table.

FIG. 12(A-B) shows Supplementary Table 7 in accordance with the present disclosure. FIG. 12A and FIG. 12B are consecutive sheets of the table.

FIG. 13(A-C) shows Supplementary Table 8 in accordance with the present disclosure. FIG. 13A, 13B, and FIG. 13C are consecutive sheets of the table.

FIG. 14(A-B) shows Supplementary Table 9 in accordance with the present disclosure. FIG. 14A and FIG. 14B are consecutive sheets of the table.

FIG. 15(A-B) shows Supplementary Table 10 in accordance with the present disclosure. FIG. 15A (15A-1 through 15A-4) shows the first section of Supplementary Table 10. FIG. 15B shows the second section of Supplementary Table 10.

FIG. 16(A-B) shows Supplementary Table 11 in accordance with the present disclosure. FIG. 16A and FIG. 16B are consecutive sheets of the table.

FIG. 17 shows Supplementary Table 12 in accordance with the present disclosure.

FIG. 18 shows Supplementary Table 13 in accordance with the present disclosure.

FIG. 19 shows Supplementary Table 14 in accordance with the present disclosure.

FIG. 20(A-C) shows Supplementary Table 15 in accordance with the present disclosure. FIG. 20A (20A-1 through 20A-9) shows the first section of Supplementary Table 15. FIG. 20B (20B-1 through 20B-9) shows the second section of Supplementary Table 15. FIG. 20C (20C-1 through 20C-6) shows the third section of Supplementary Table 15.

FIG. 21 shows Supplementary Table 16 in accordance with the present disclosure.

### DETAILED DESCRIPTION OF THE INVENTION

The present disclosure is based, at least in part, on the discovery of proteomic profiles predictive of Alzheimer's disease (AD) status. As shown herein, inventors not only identified proteomic profiles for sporadic AD, but also for individuals with AD-risk variants in TREM2 and pathogenic variants in APP and PSEN1/2. These proteomic profiles enabled the creation of tissue-specific prediction models and the identification of causal proteins and pathways for sporadic and genetically defined AD subtypes.

CSF and plasma molecular signatures of AD phenotypes have been identified by analyzing a range of AD phenotypes using well-characterized datasets containing. These identified panels of proteins from CSF and plasma are capable of predicting AD status with sensitivity and selectivity as well as, or better than, the well accepted and validated CSF As and tau biomarkers.

A set of 9 proteins in CSF and 12 in plasma have been identified that predict sporadic disease status.

A set of 7 proteins in CSF and 9 in plasma have been identified in a larger and longitudinal data set that predict TREM2 risk variant carrier status.

A set of 17 proteins in CSF and plasma has been identified that predicts autosomal dominant AD.

These biomarker panels contain enough proteins to provide good predictive power, yet are simple enough to be clinically manageable and not prohibitively expensive to measure. The CSF panel provides superior predictive power compared to existing biomarkers, and the plasma panel provides similar power from a much more accessible biospecimen that can allow for frequent testing or population-level screening. Since they are not tied to pTau/Aβ42, they will provide a method of tracking disease progress taking medications targeting Tau and/or As and may potentially detect disease earlier.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes.

(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to, samples or reagents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit, instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Multi-Tissue Proteomics Identifies Molecular Signatures for Sporadic and Genetically Defined Alzheimer's Disease Cases

This Example describes multi-tissue proteomic characterization of sporadic and genetically defined Alzheimer's disease (AD) cases.

Abstract

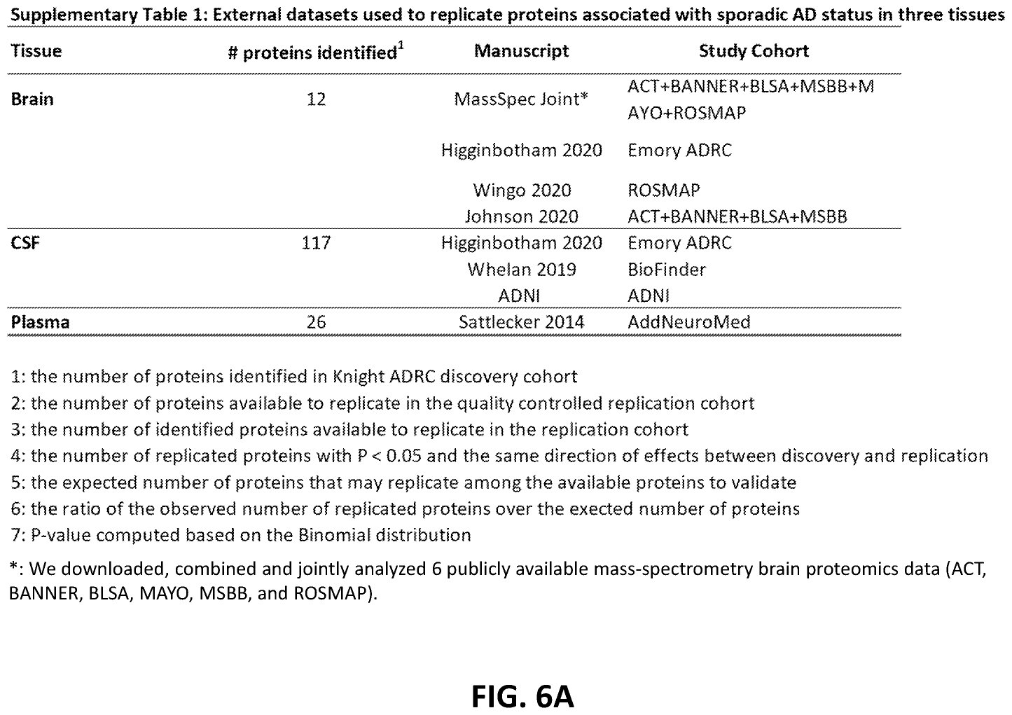

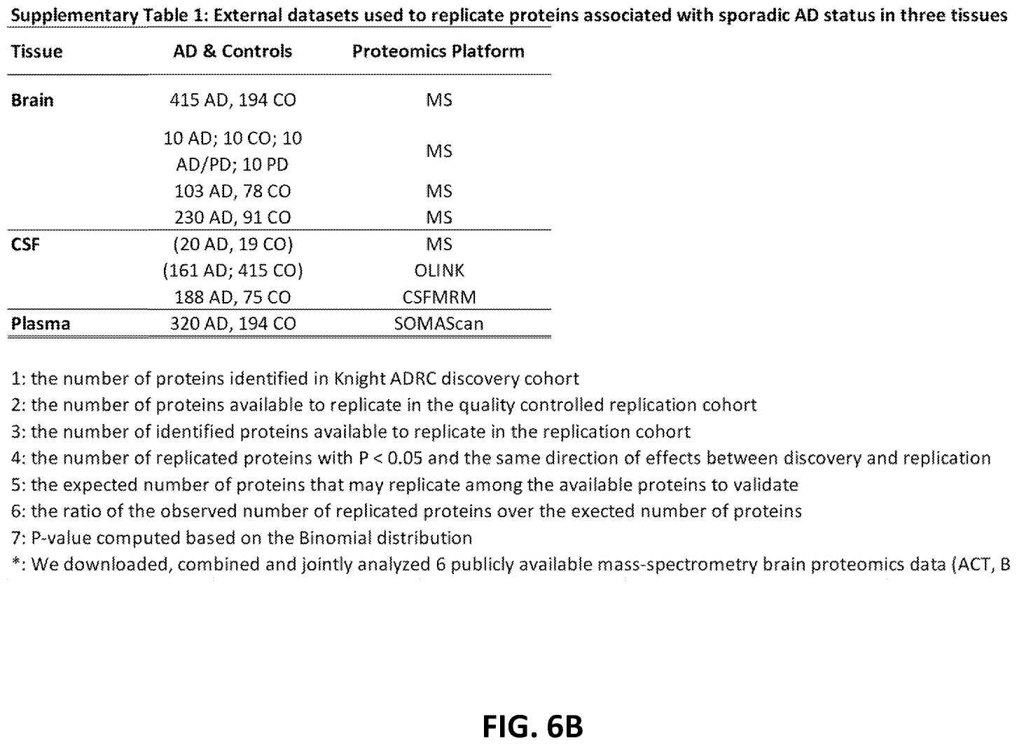

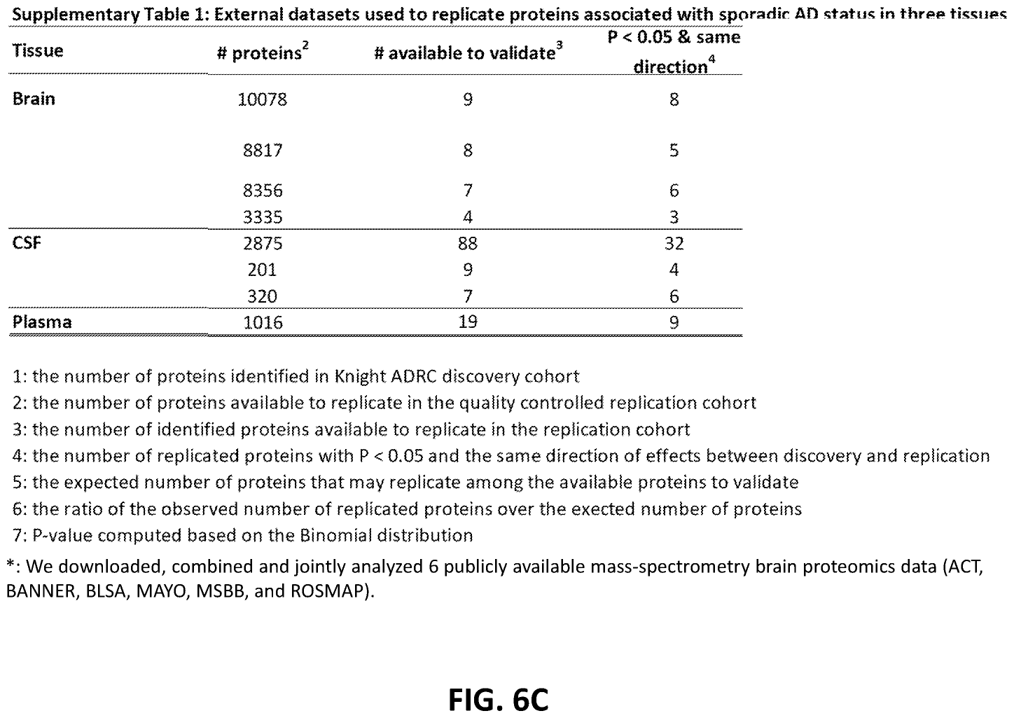

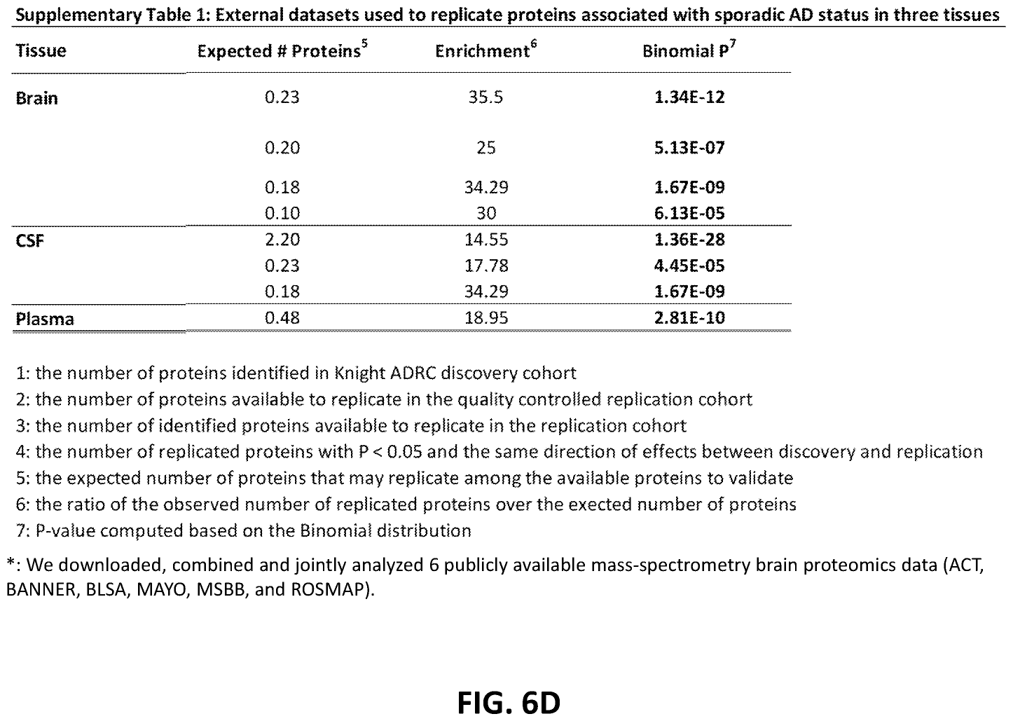

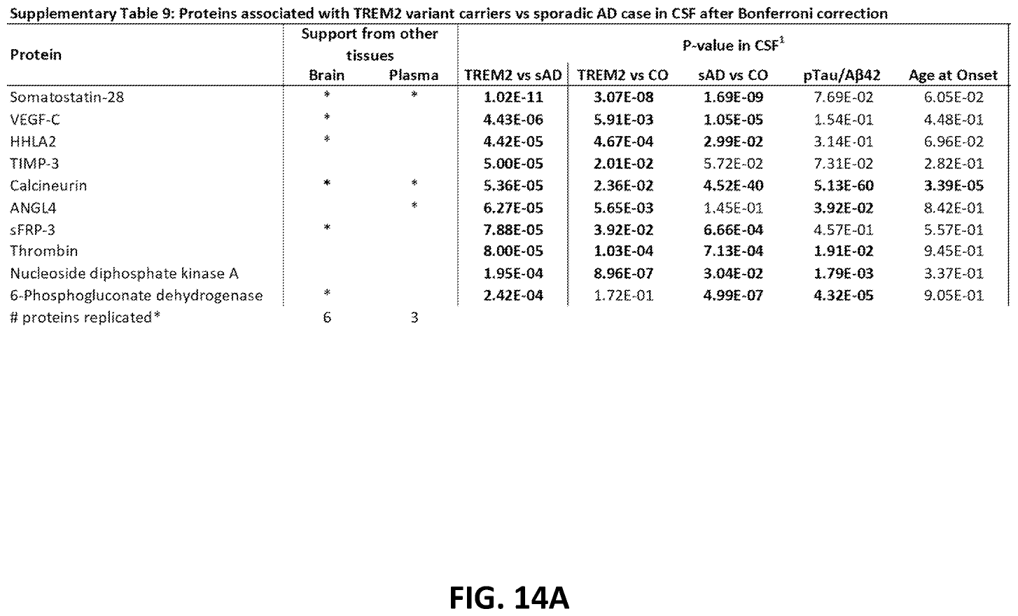

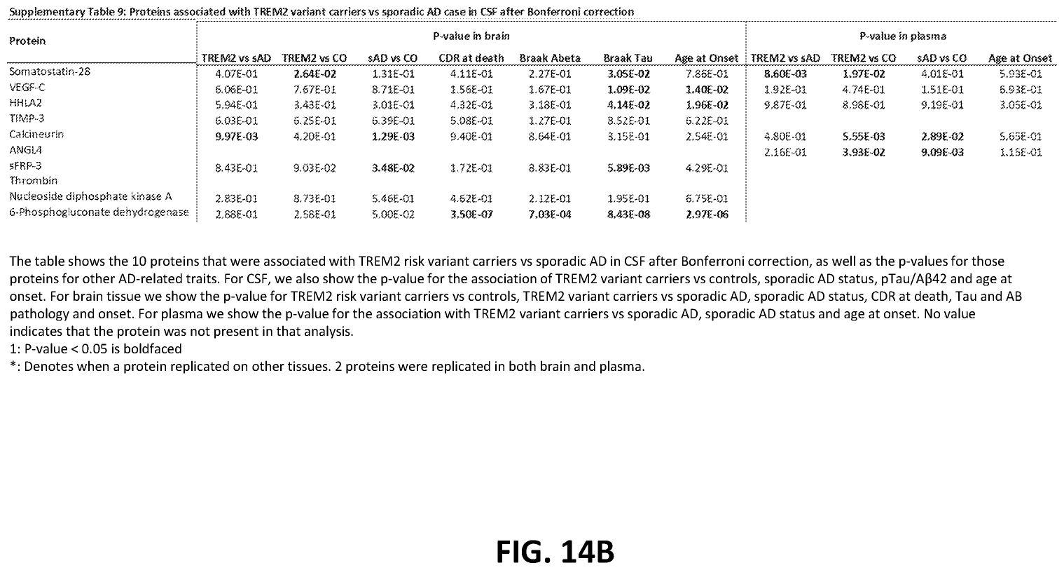

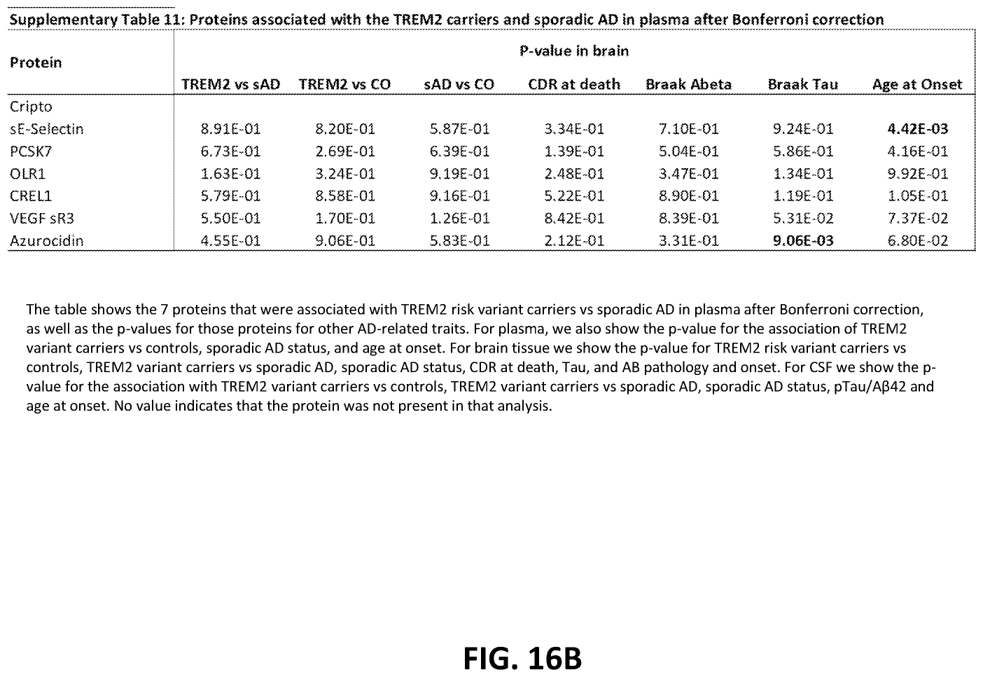

Alzheimer's disease (AD) is a heterogeneous disease, and many genes are associated with AD risk. Most proteomic studies, while instrumental in identifying AD pathways and genes, focus on single tissues and sporadic AD cases. Multi-tissue proteomic signatures for sporadic and genetically defined AD (e.g., pathogenic variant carriers in APP and PSEN1/2 and risk variant carriers in TREM2) will illuminate the biology of this heterogeneous disease. Described herein is one of the largest multi-tissue proteomic profiles to date, based on 1,305 proteins in brain (n=360), cerebrospinal fluid (CSF; n=717), and plasma (n=490) from the Knight Alzheimer Disease Research Center (Knight ADRC) and Dominantly Inherited Alzheimer Network (DIAN) cohorts. Proteomic signatures in brain, CSF, and plasma were identified for sporadic AD status, and these findings were replicated in multiple, independent datasets (see Supplementary Tables 1-16 in FIGS. 6-21). The area under the curve (AUC) for CSF proteins was 0.89 in discovery and 0.90 in the replication dataset, which was significantly higher than the AUC for CSF p-tau181/Aβ42 (AUC=0.81; P=2.4×10⁻⁶). A specific proteomic signature was also identified for TREM2 variant carriers that differentiated TREM2 variant carriers from sporadic AD cases and controls with high sensitivity and specificity (AUC=0.81-1). In addition, the proteins that showed differential levels in sporadic AD were also altered in autosomal dominant AD, but with greater effect size (1.4 times, P=3.8×10⁻⁵), and proteins associated with autosomal dominant AD, in brain tissue also replicated on CSF (p=1.36×10⁻⁹). Enrichment analyses highlighted several pathways including AD (calcineurin, APOE, GRN), Parkinson's disease (α-synuclein, LRRK2), and innate immune response (SHC1, MAPK3, SPP1) for the sporadic AD or TREM2 variant carriers. These findings show the power of multi-tissue proteomics' contribution to the understanding of AD biology and to the creation of tissue-specific prediction models for individuals with specific genetic profiles, ultimately supporting its utility in creating individualized disease risk evaluation and treatment.

Introduction

Alzheimer's disease (AD), the most common cause of dementia, is a heterogeneous neurodegenerative disease characterized by neuronal loss, neuroinflammation, and memory decline. Clinical sequelae reduce quality of life and cost the healthcare system up to $244 billion annually in the US, with additional impact on caregivers' emotional distress. Proteomic studies have been instrumental in identifying biomarkers and pathways implicated in AD, but most have been limited to single tissues and only differentiate between sporadic AD cases and controls. Deep molecular characterization of controls, sporadic AD cases and genetically defined AD subtypes, such as individuals carrying pathogenic mutations in amyloid-beta precursor protein (APP) and presenilin genes (PSEN1 and PSEN2) or high-effect risk variants in triggering receptor expressed on myeloid cells 2 (TREM2), is critical for fully understanding the biology of this heterogeneous disease and for identifying novel molecular biomarkers and therapeutic targets. Herein is described the results of multi-tissue, high-throughput deep proteomic profiling. Proteins associated with AD status that replicated in external datasets were identified. Not only were proteomic profiles identified for sporadic AD, but also for individuals with AD-risk variants in TREM2 and pathogenic variants in APP and PSEN1/2. These proteomic profiles enabled the creation of tissue-specific prediction models and the identification of causal proteins and pathways for sporadic and genetically defined AD subtypes.

Study Design

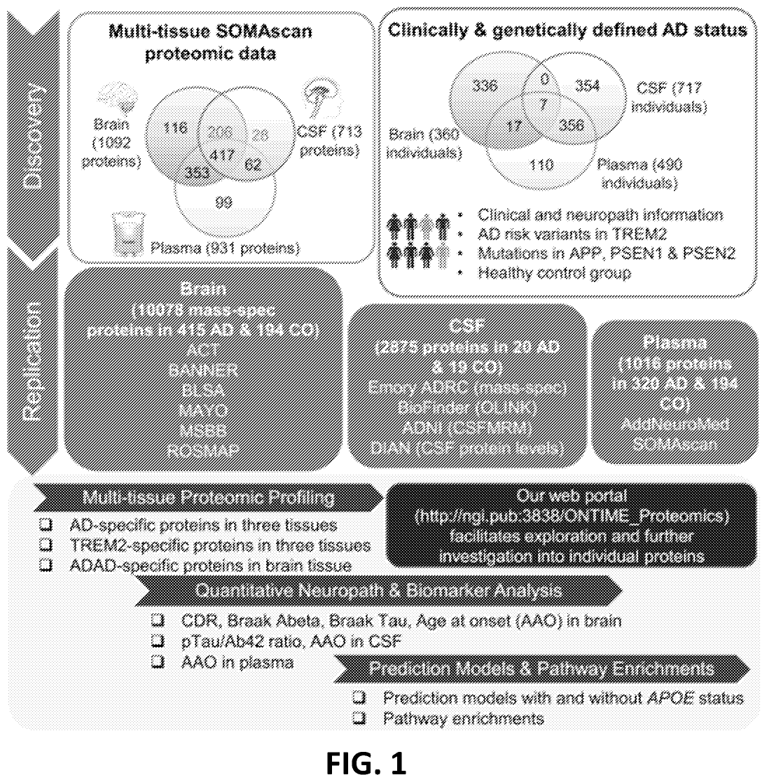

To elucidate the downstream effects of genes and the functional mechanisms associated with AD, high-throughput, deep proteomic profiles were generated using SOMAscan targeting 1,305 proteins in brain tissue, cerebrospinal fluid (CSF), and plasma (FIG. 1). These neurologically relevant tissues were obtained from well-characterized individuals with comprehensive clinical information about AD pathology and cognition in the Knight ADRC and DIAN. After stringent quality control (QC) and data cleaning, a total of 1,092 proteins from 360 brain tissues remained. These brain proteomic data include 24 individuals carrying autosomal dominant AD (ADAD) mutations in APP and PSEN1/2, 290 individuals with autopsy-confirmed AD, 21 TREM2 variant carriers, and 25 cognitively normal individuals with no significant brain pathology (Table 1, below). CSF data contained 713 proteins from 176 individuals with a clinical diagnosis of AD, 47 TREM2 variant carriers, and 494 cognitively normal individuals. Plasma data contain 931 proteins from 105 individuals with a clinical diagnosis of AD, 131 TREM2 variant carriers, and 254 cognitively normal individuals (FIG. 1).

TABLE 1
Summary characteristics of participants with proteomic
measures in the Knight ADRC and DIAN cohorts.

| Tissue | Status | Sample size (N) | % Female | Age (mean ± SD) |
|---|---|---|---|---|
| Brain | CO | 25 | 61.72 | 88.24 ± 8.85 |
| | AD | 290 | 33.33 | 83.98 ± 8.83 |
| | ADAD | 24 | 76.00 | 55.67 ± 14.58 |
| | TREM2 | 21 | 57.14 | 82.57 ± 7.62 |
| CSF | CO | 494 | 55.26 | 73.15 ± 6.43 |
| | AD | 176 | 46.02 | 74.60 ± 7.02 |
| | TREM2 | 47 | 44.68 | 74.00 ± 6.48 |
| Plasma | CO | 254 | 57.48 | 71.53 ± 7.31 |
| | AD | 105 | 37.14 | 72.59 ± 7.67 |
| | TREM2 | 131 | 64.89 | 74.98 ± 8.17 |

CO = healthy control; AD = sporadic AD cases; ADAD = Autosomal dominant AD; TREM2 = AD-risk variant (p.E151K, p.H157Y, p.L211P, p.R136Q, p.R163Q, p.R47H, p.R62H, p.T96K) carriers in TREM2; CSF = cerebrospinal fluid.

AD status was defined based on neuropathological examination for those samples with brain autopsy and clinical examination for those with CSF and plasma tissue. In this study, proteins were identified with different levels in clinical AD cases vs. controls and not based on biomarker levels or the ATN framework, which combines the amyloid-3 pathway (A), tau-mediated pathophysiology (T), and neurodegeneration (N), because one of the goals of this study was to compare the performance of the prediction models generated in this study with these well-accepted and validated CSF biomarkers (Aβ and p-tau181).

To validate and replicate proteins that were associated with AD, TREM2 risk variant carriers or ADAD mutation carrier status, two approaches were followed: first, for sporadic AD and TREM2 risk variant carriers, the common set of proteins dysregulated in the three tissues (brain, CSF, and plasma) were identified. For ADAD, only high-throughput proteomic screening was performed on brain tissue. Those proteins that were associated with ADAD status in brain were analyzed in CSF from 289 ADAD mutation carriers and 184 non carriers from the DIAN study. Second, for sporadic AD, seven publicly available datasets were used to replicate findings (Supplementary Table 1 in FIG. 6(A-D)). For brain, the mass-spectrometry data for the following 6 studies was downloaded: the Adult Changes in Thought (ACT), Banner Sun Health Research Institute (BANNER), Baltimore longitudinal study of aging (BLSA), Mayo Clinic (MAYO), Mount Sinai Brain Bank (MSBB), the Religious Orders Study and the Memory and Aging Project (ROSMAP). Differential abundance analysis was then performed jointly for a total of 10,078 proteins measured in 415 AD patients and 194 controls, called hereafter MassSpec Joint. For CSF, Alzheimer's Disease Neuroimaging Initiative (ADNI) multiple reaction monitoring (MRM) proteomic data containing 320 proteins in 263 samples was obtained and analyzed. Results based on BioFinder OLINK data and Emory-ADRC mass-spectrometry data were also used. For plasma, differential analysis was performed on the AddNeuroMed SOMAscan 1.1K proteomic data. Public datasets were not used to replicate the proteins dysregulated in TREM2 or ADAD mutation carriers because there were not enough carriers in public datasets. Finally, the replicated proteins were used to generate prediction models and run pathway analyses. The results from this study were combined with recent pQTL, colocalization, and Mendelian randomization findings to identify causal proteins.

Multi-Tissue Proteomic Signatures of AD

Sporadic AD Cases

To identify multi-tissue proteomic signatures for clinical AD, differential analysis were performed with a subgroup of sporadic AD patients and healthy individuals in each of the three tissues, independently. Specifically, a surrogate variable analysis (SVA)14 was performed to remove batch effects and other unmeasured heterogeneity in all three proteomic datasets. Regression analysis were then performed of log-transformed protein abundance levels as a dependent variable and sporadicAD status as an independent variable while considering age, sex, and SVA as covariates.

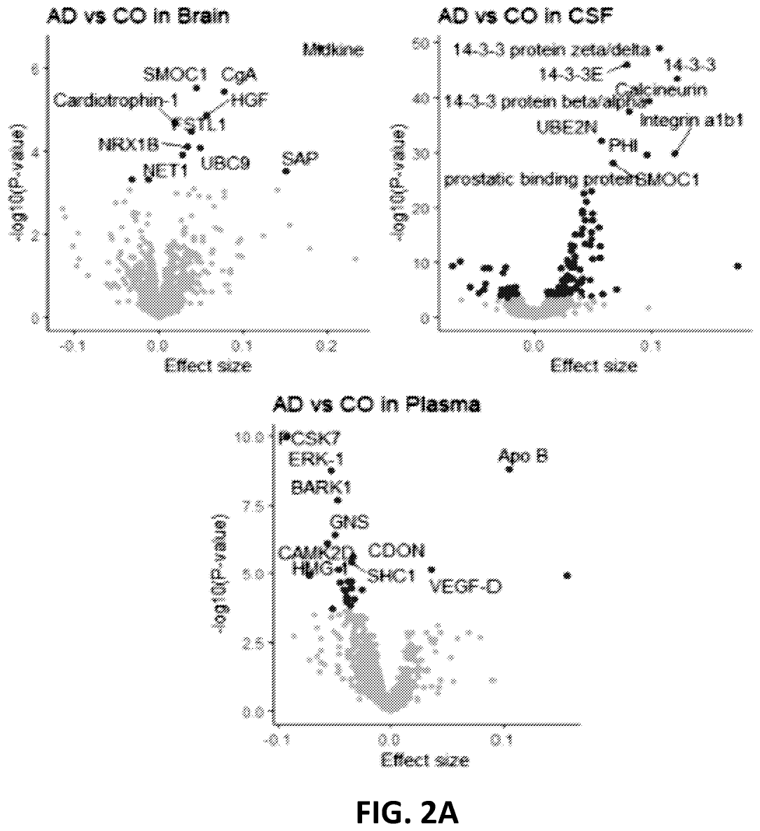

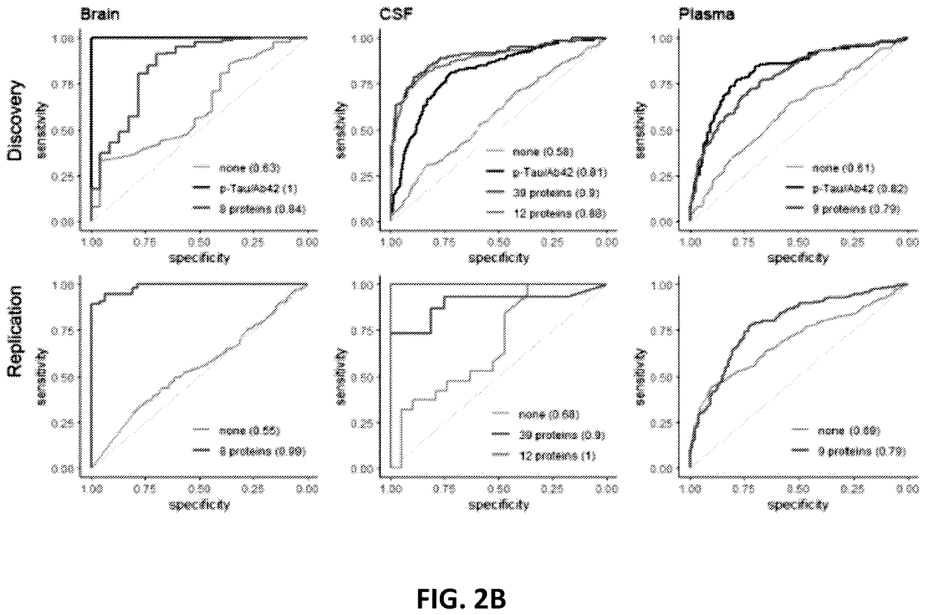

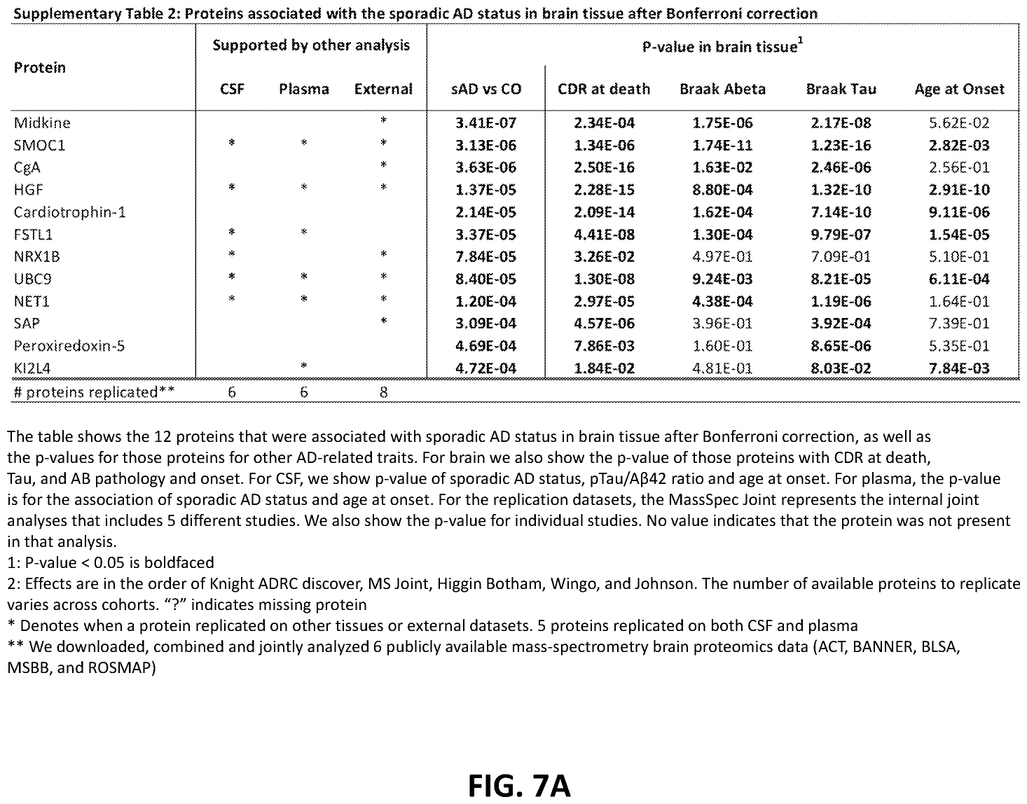

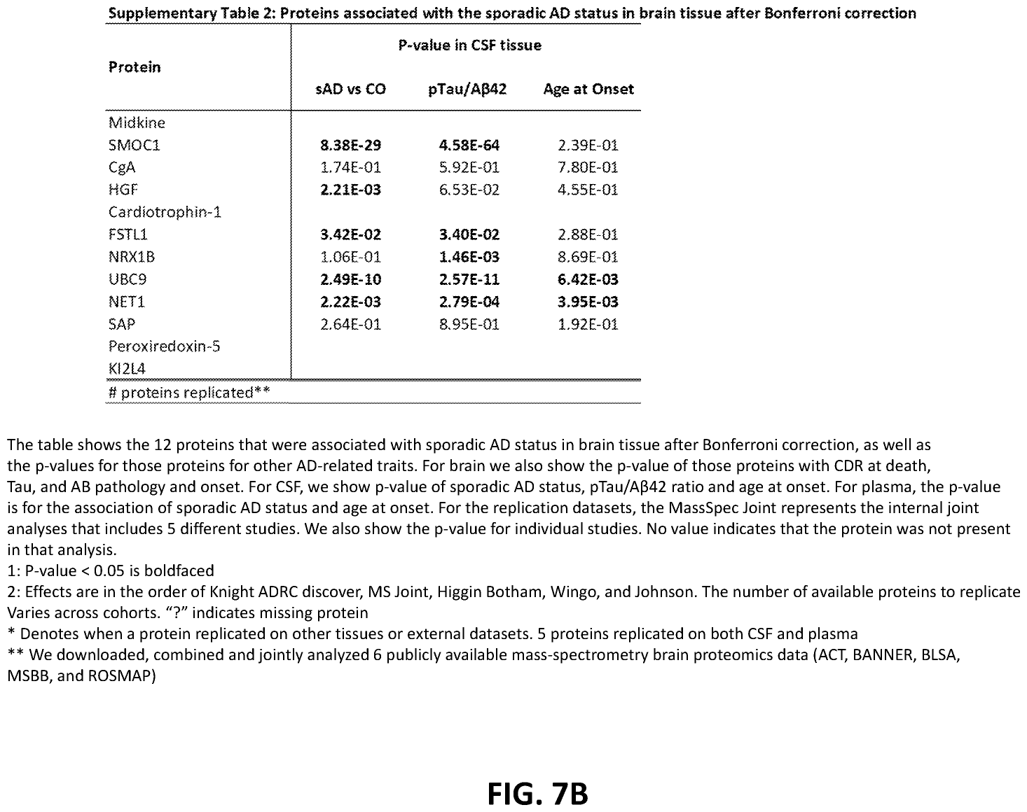

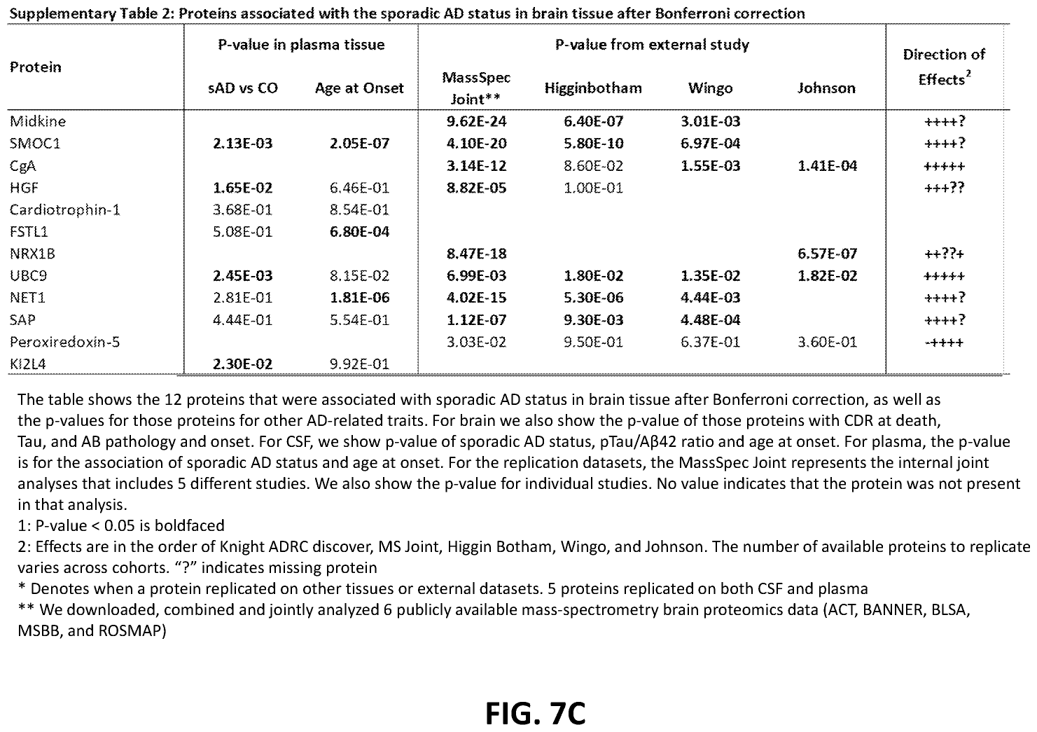

Brain proteomic profiles for sporadic AD. In the brain, proteins showed significant association for AD status after Bonferroni correction (FIG. 2A and Supplementary Table 2 in FIG. 7(A-C)). The Bonferroni-corrected threshold was chosen as it is more conservative than false discovery rate (FDR). All 12 proteins were nominally significant (P<0.05) with other AD-related phenotypes including age at onset and AD neuropathology characteristics such as Braak scores and CDR at death (FIGS. 1, 2A, 2B, and Supplementary Table 2 in FIG. 7(A-C)). As proteomic data from CSF and plasma was available, which proteins are also associated with AD risk or onset were determined in these other two tissues. Given low overlap (FIG. 1) in individuals who have proteomic data across tissues, this was used as an internal validation. By leveraging across-tissue data, any tissue-specific signal will not replicate. One caveat of using the multi-tissue data is that not all proteins passed QC across all three tissues. Among the 12 proteins associated with AD status in brain, only 6 were found in both CSF and plasma. Of these, 5 proteins (SMOC1, HGF, FSTL1, UBC9, and NET1) were associated with AD status or age at onset in both CSF and plasma data (P<0.05, Supplementary Table 2 in FIG. 7(A-C)), which represents an enrichment of 333-fold (P=5.8×10⁻¹³) to what would be expected by chance.

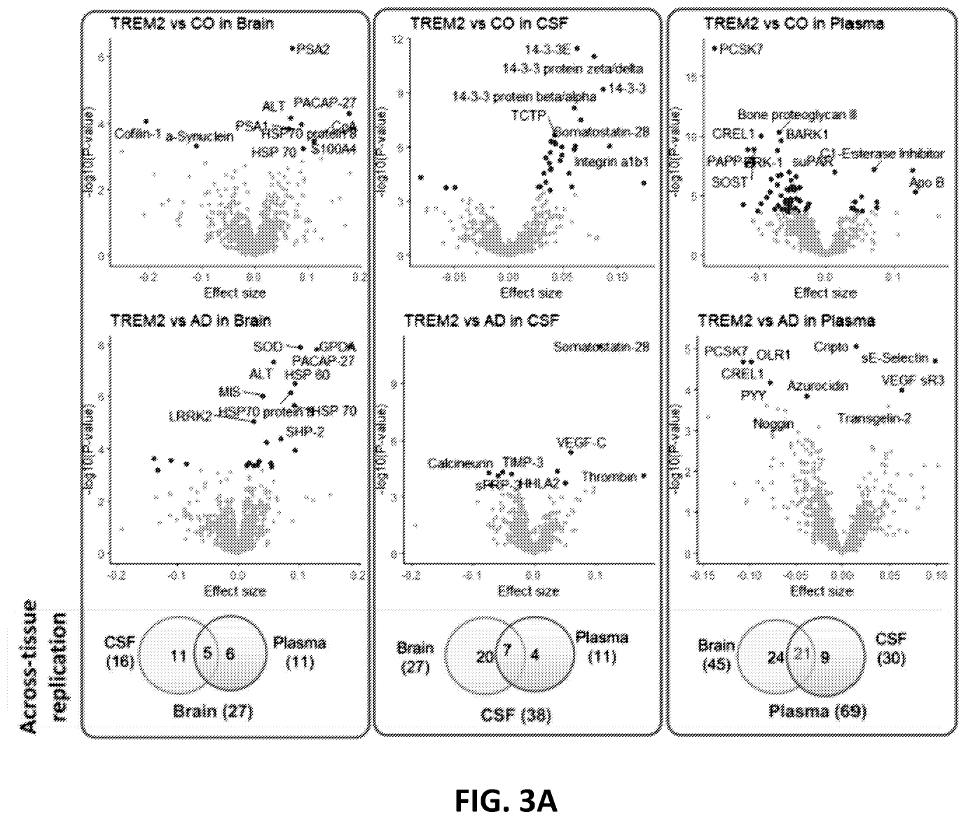

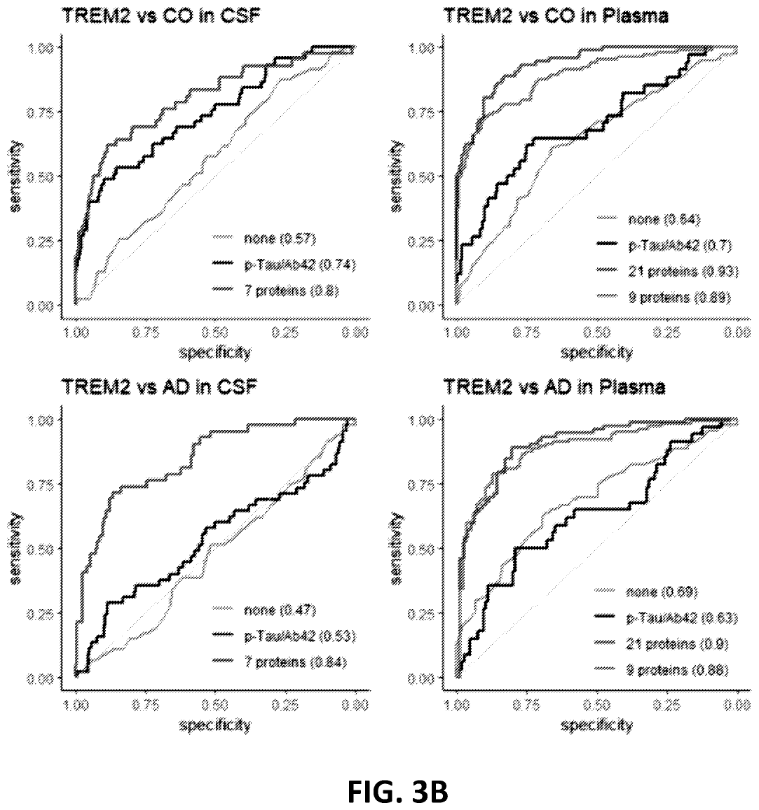

To externally replicate these findings, the merged mass-spectrometry brain data (MassSpec Joint) that includes 10,078 proteins from 415 AD patients and 194 controls was used, and association analyses with AD status were performed. As the proteomic data available in these studies were generated using a different platform, all 12 proteins that were significant in the discovery data were not able to be tested. Of the nine proteins that were present in these datasets, 8 replicated (Midkine, SMOC1, CgA, HGF, NRX1B, UBC9, NET1, and SAP) with P<0.05 and in the same direction of effect. This represents an enrichment of 35-fold to what would be expected by chance (P=1.3×10⁻¹²). In addition, to confirm that the results were not false positives due to the joint analysis that included all 6 studies, additional analyses were performed in each study (Johnson et al., Higginbotham et al., and Wingo et al.). Individual study analyses also provided enrichments of 25-34-fold (Supplementary Table 1 in FIG. 6(A-D)). A significant correlation was also found in the effect size for the association of the proteins with AD status between the discovery results and the merged replication results (MassSpec Joint) (P<3.6×10⁻³; FIG. 3A). Together, these results indicate that the brain proteomic signature identified herein replicates in external independent samples and is extremely robust across orthogonal proteomics platforms.

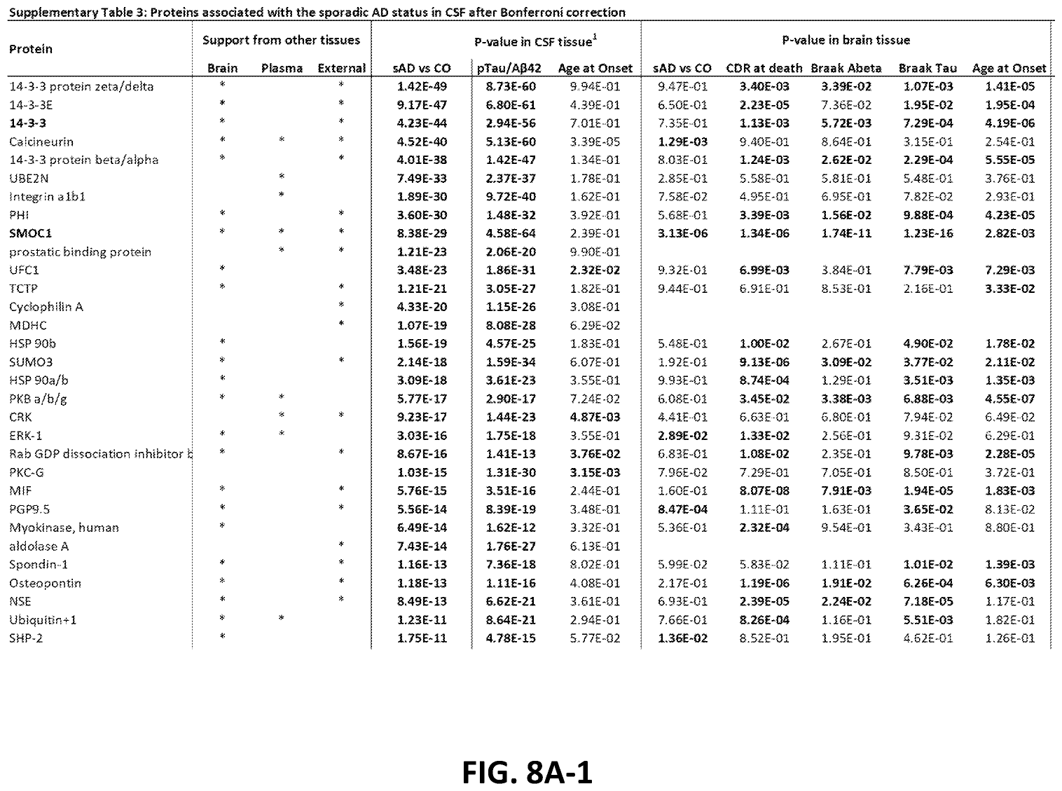

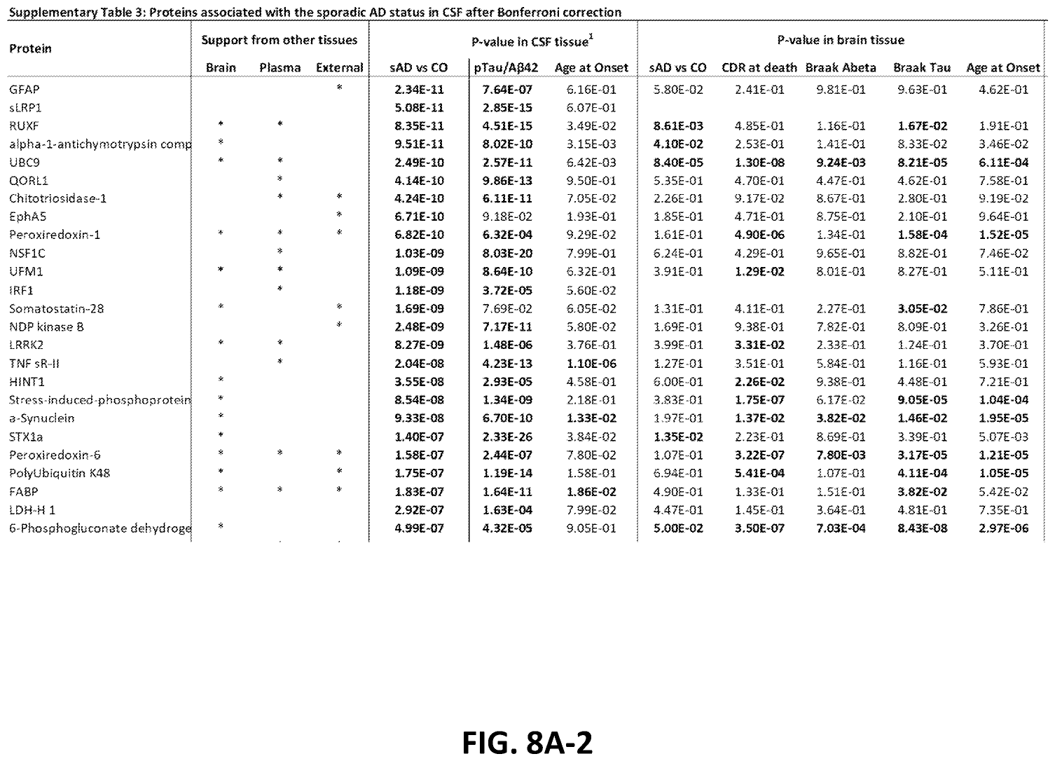

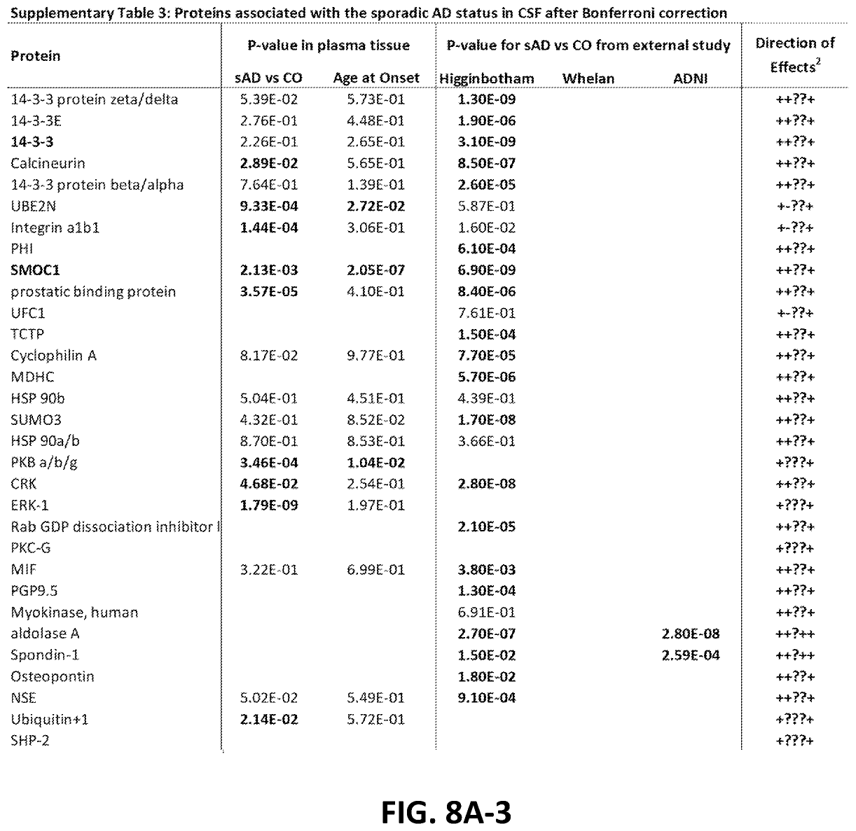

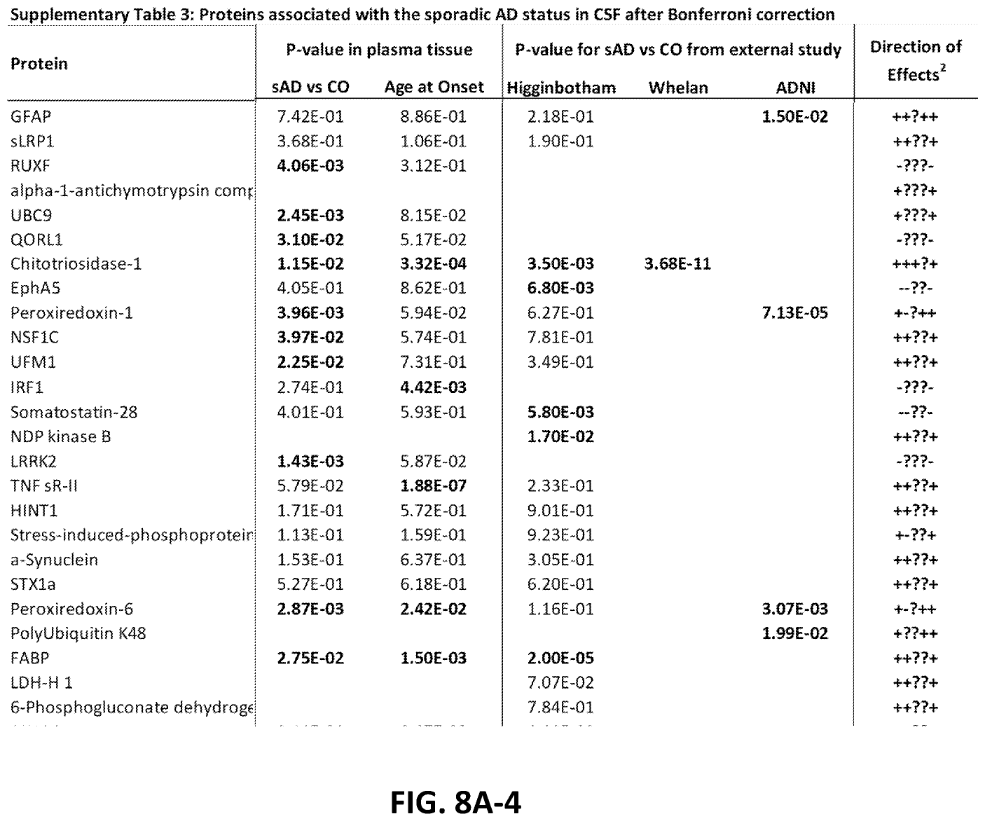

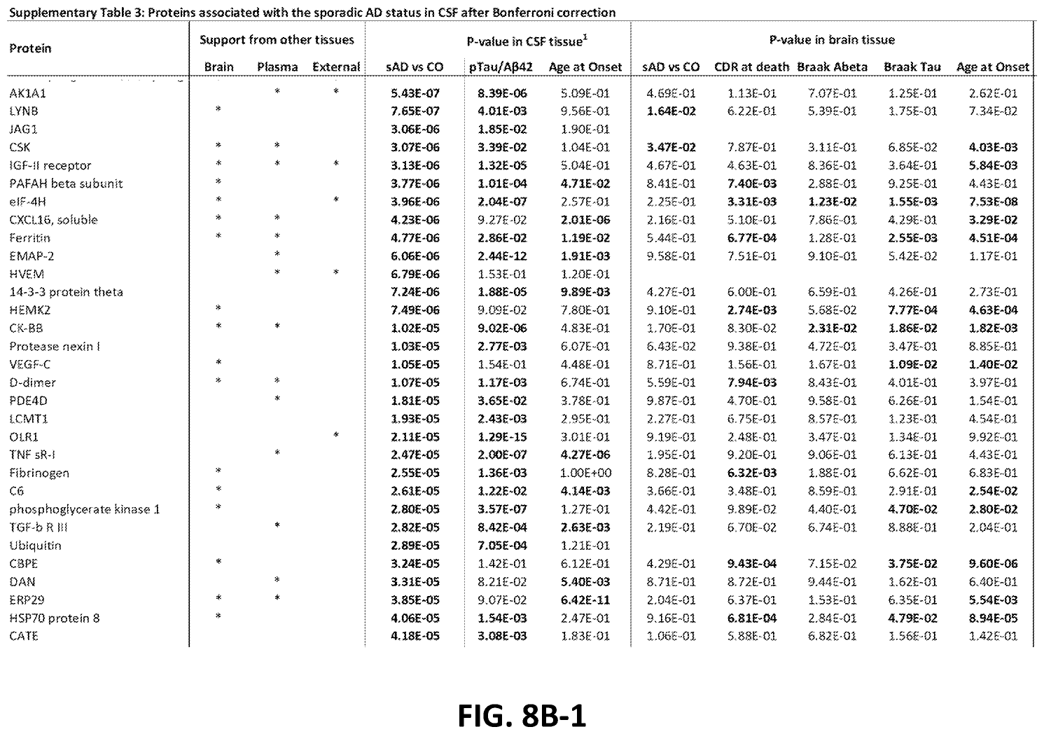

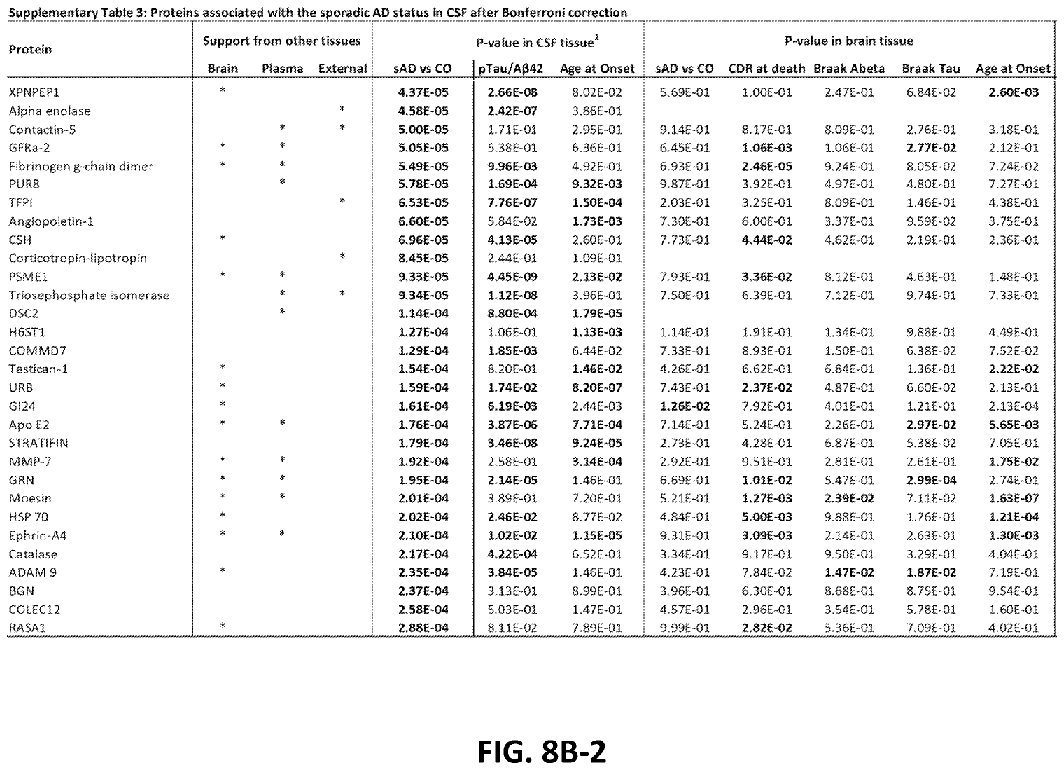

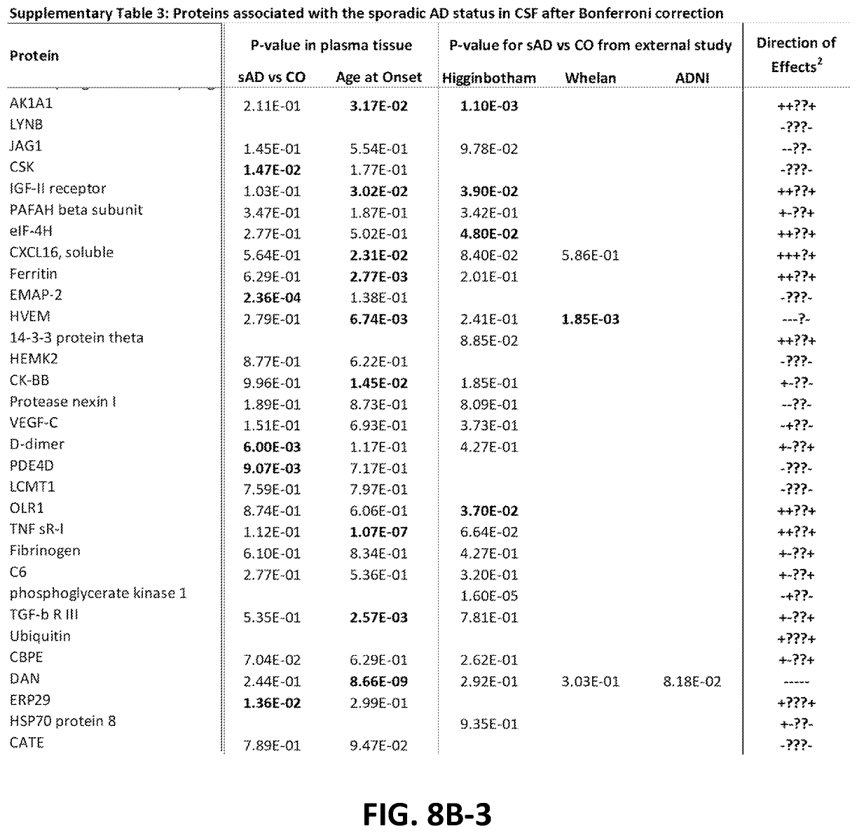

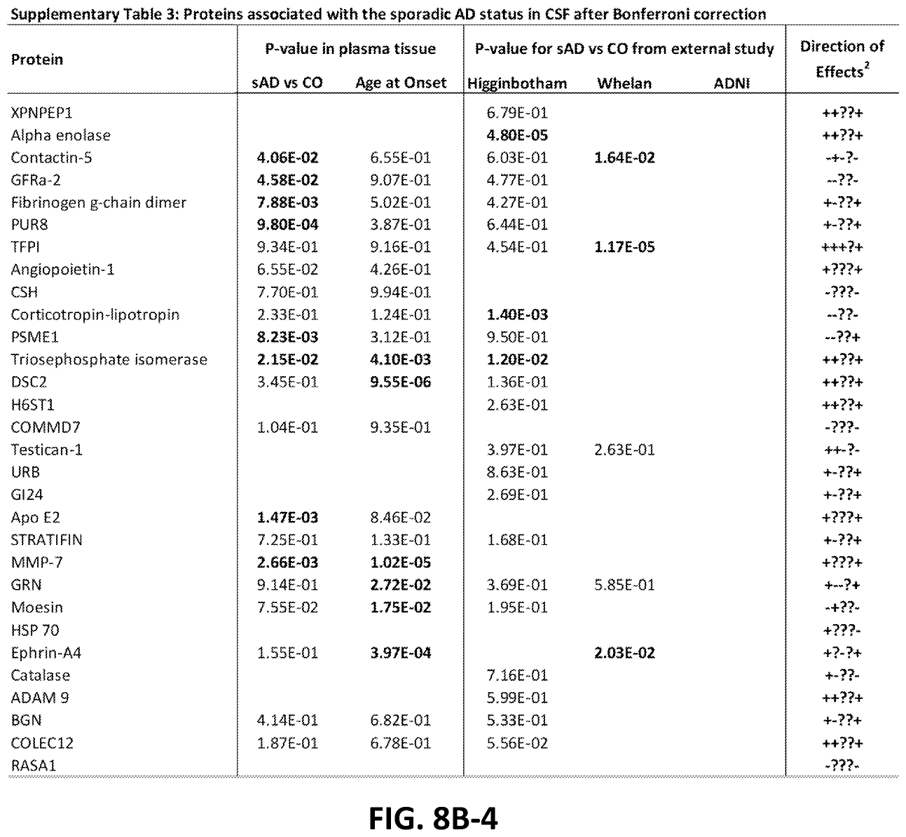

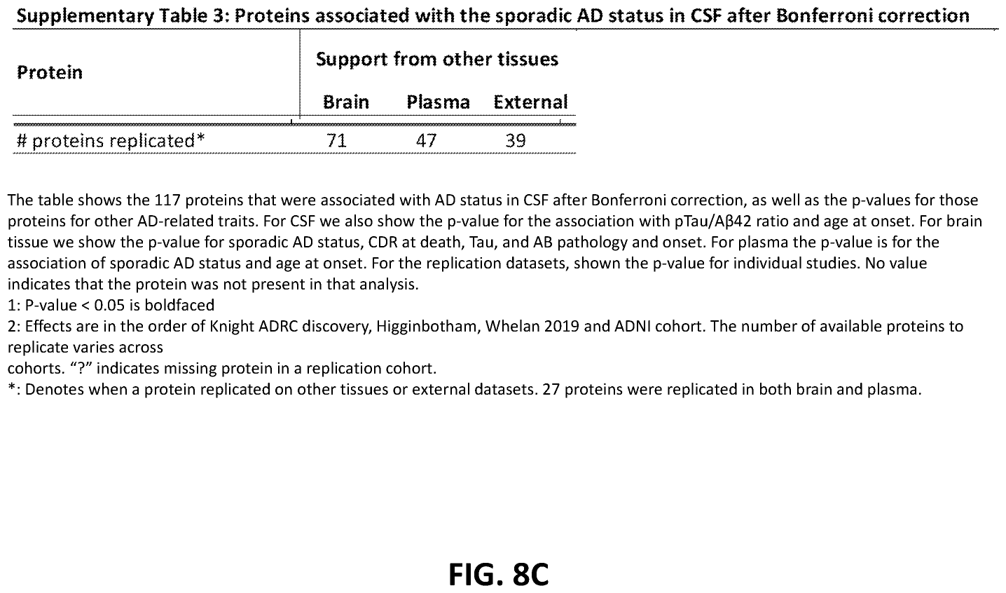

CSF proteomic profiles for sporadic AD. In CSF, 117 proteins were associated with clinical AD status after Bonferroni correction (FIG. 2A, Supplementary Table 3 in FIG. 8(A-C)). Of these 117 proteins, 78 passed QC in brain and plasma tissues, and 27 proteins (including ERK-1 and LRRK2) replicated in both tissues (138-fold enrichment, P=3.3×10⁻⁵). An additional 44 proteins replicated in brain and 16 in plasma. To externally replicate the identified proteins in CSF, Alzheimer's Disease Neuroimaging Initiative (ADNI) multiple reaction monitoring (MRM) proteomic data containing 320 proteins in 263 samples was analyzed. In addition, results based on BioFinder OLINK data of 201 proteins in 576 samples presented by Whelan et al. and from the mass-spectrometry-based Emory-ADRC study that includes 2,875 proteins in just 40 samples presented by Higginbotham et al were obtained. Of the 117 CSF proteins identified in this study, 90 were present in these external datasets. Of these, 39 proteins (including 14-3-3, Calcineurin, SMOC1, GFAP, SPP1, and Peroxiredoxin-1) replicated in the same direction (14- to 34-fold enrichments, P≤4.4×10⁻⁵). The major overlap in the number of proteins with the data herein is the Emory-ADRC study, which only includes 40 samples. Therefore, the power to replicate the initial findings is limited. A larger number of proteins is expected to replicate in larger studies.

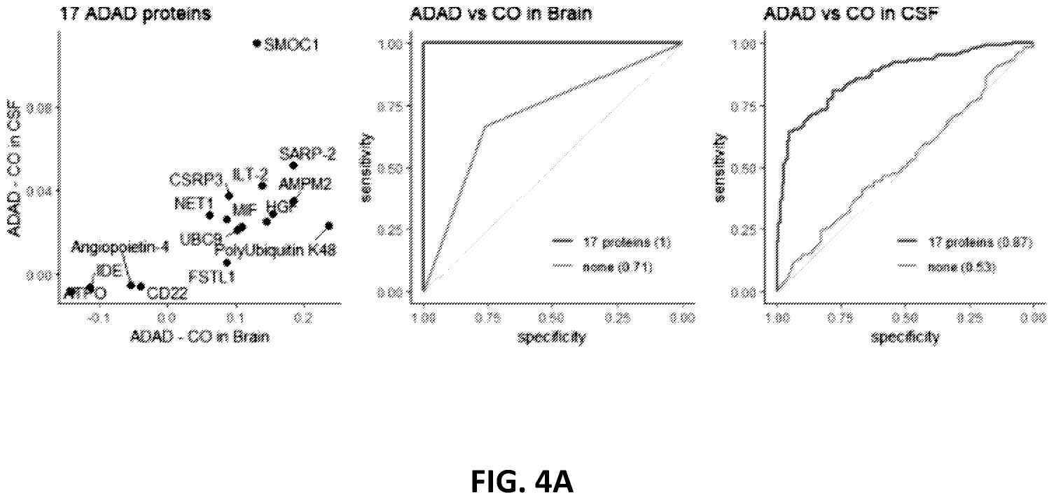

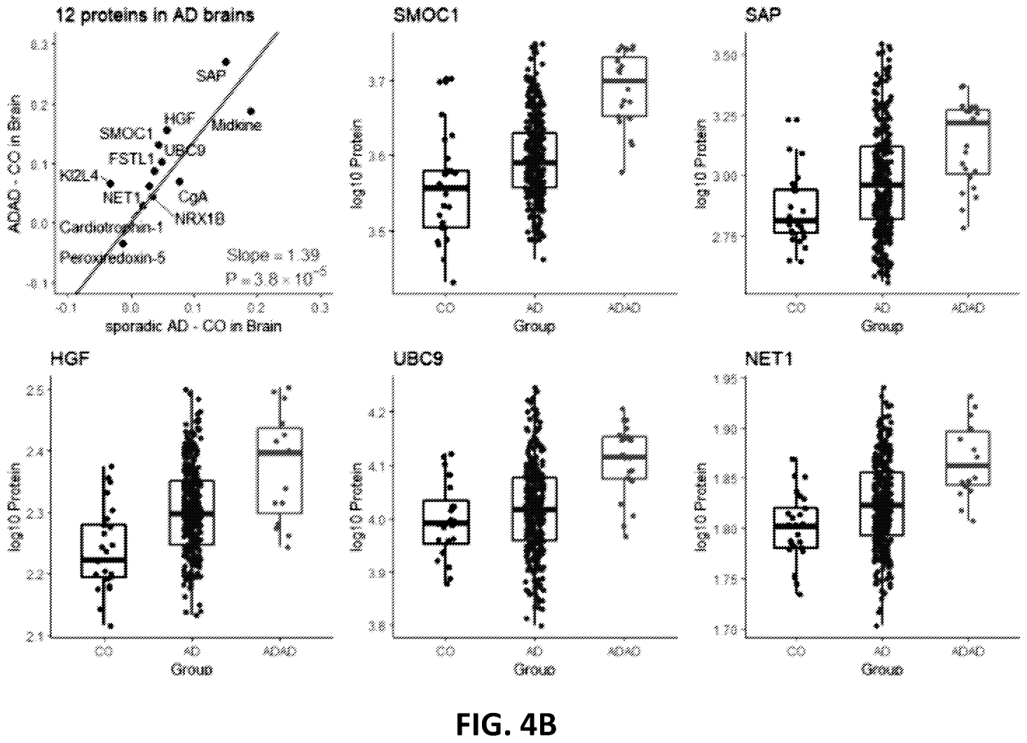

Several studies have demonstrated that up to 30% of cognitively normal elderly individuals could be pre-symptomatic for AD and that other neurodegenerative diseases can masquerade, clinically, as AD dementia. Therefore, clinically defined case-control status may not be the best phenotype for novel biomarker discovery. It has been proposed that biomarker-based categorization provides a more powerful approach to identify proteins altered in AD. CSF Aβ42 and p-tau levels are one of the best fluid biomarkers identified to date for distinguishing pathology-free controls from AD dementia and several studies have demonstrated that CSF p-tau/Aβ42 ratio is a marker not only for AD status but also for predicting AD progression from normal to dementia within 5 years. As CSF p-tau/Aβ42 was available for most samples with CSF (689 out of 720), a regression analysis of protein levels was also performed considering p-tau/Aβ42 ratio as a predictor. 92 proteins were found that were significant for p-tau/Aβ42 ratio at Bonferroni-corrected threshold. Of the 117 proteins associated with clinical AD status, 74 were significant for CSF p-tau/Aβ42 at Bonferroni-corrected threshold and the remaining were nominally significant. In fact, a very strong correlation (R²=0.86 and P<1.0×10⁻¹⁶; FIG. 4A and FIG. 4B) of the effect was found across all 713 QCed proteins between the two analyses. This indicates that using case-control status for the Knight ADRC is highly accurate and leads to the similar results as using biomarker-defined case-control status.

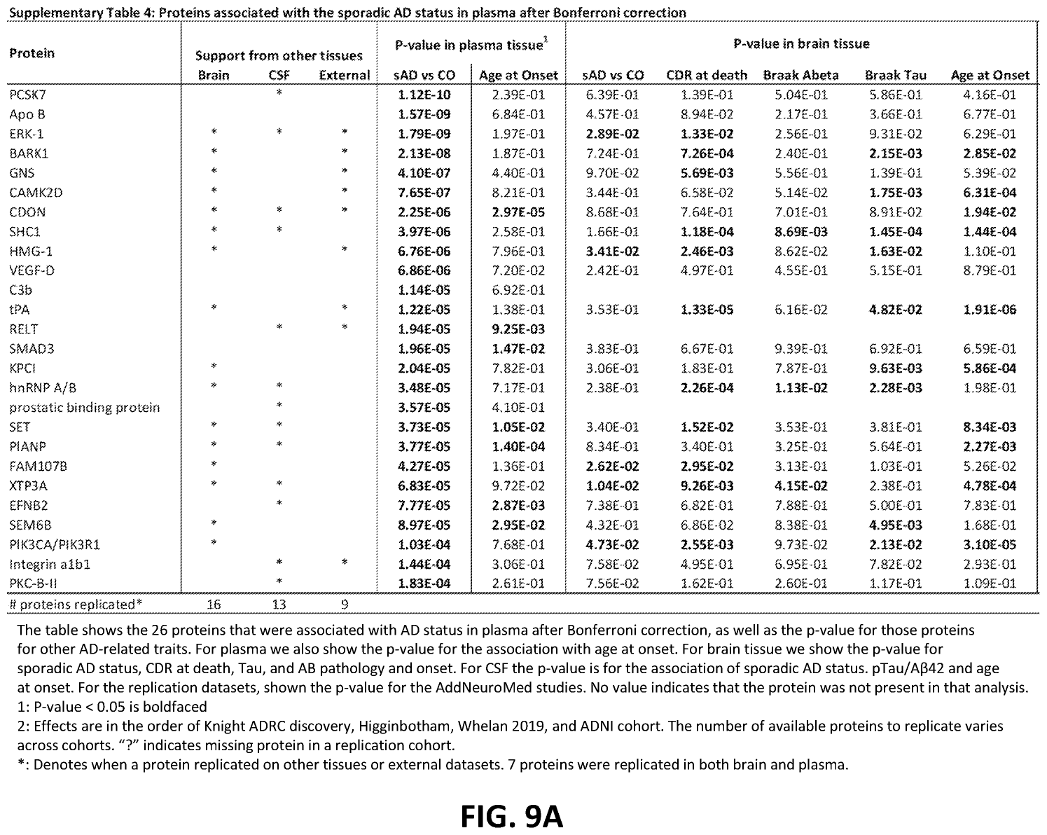

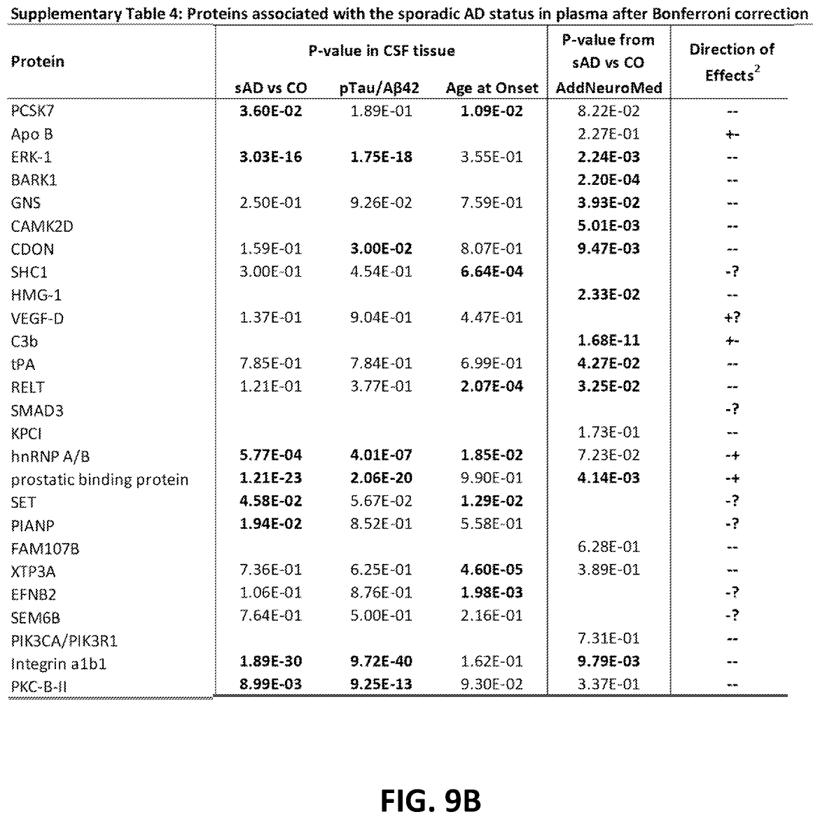

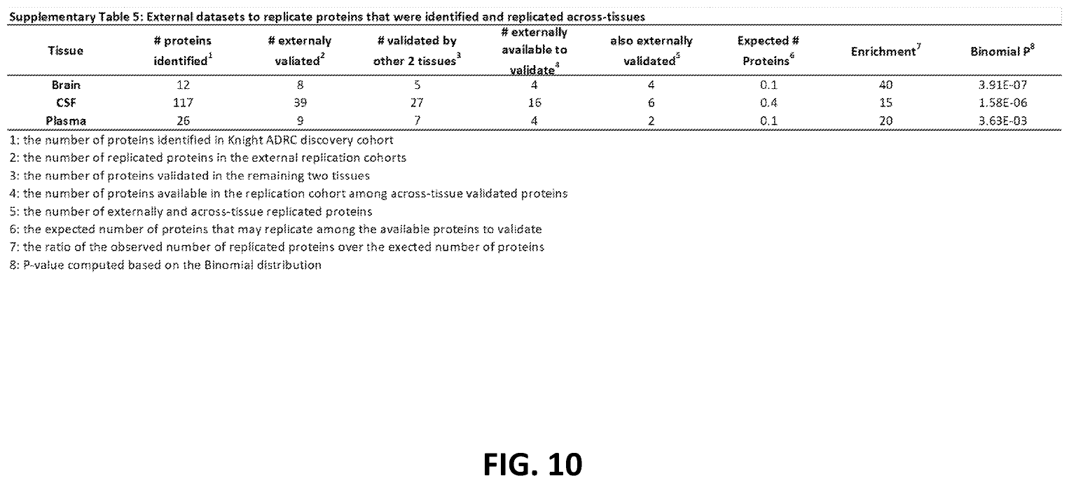

Plasma proteomic profiles for sporadic AD. In plasma, 26 proteins were associated with sporadic AD status after Bonferroni correction (FIG. 2A, Supplementary Table 4 in FIG. 9(A-B)). Similar to previous analyses, the multi-tissue data was leveraged to replicate these findings. Of the 26 plasma proteins associated with AD status, 16 passed QC in brain and CSF and seven proteins (including ERK-1, CDON, and SHC1) replicated (175-fold enrichment, P=6.8×10⁻¹⁵). To externally replicate the findings, the AddNeuroMed SOMAscan 1.1K proteomic data that was processed and deposited by Sattlenecker et al. was obtained and differential analysis was performed in 320 individuals with AD and 194 controls. Out of 26 proteins, 19 were tested in this dataset and 9 proteins (including CAMK2D and HMG-1) replicated (18.9-fold enrichment, p=2.8×10⁻¹⁰). In summary, 8, 39, and 9 proteins have been identified herein that are associated with AD status and replicated in several independent cohorts using orthogonal technologies in brain, CSF, and plasma, respectively. These proteins likely represent only a subset of proteins that could be associated with AD status, as not all proteins identified in this study were assayed in the replication datasets and most of the replication datasets had smaller sample sizes than the discovery data, providing limited power. Multi-tissue data was also leveraged to replicate the single-tissue findings. Sometimes, it may not be possible to use external datasets for replication, therefore an enrichment test was performed to determine whether the proteins that showed an internal cross-tissue replication would also replicate in other studies. The analyses indicate that proteins identified in each tissue and supported by the two remaining tissues were more likely to replicate in external independent datasets (15- to 40-fold enrichments, P≤3.63×10⁻³, Supplementary Table 5 in FIG. 10), suggesting that multi-tissue proteomic data may be used as a viable replication strategy.

TREM2 Risk Variant Carriers.

Several rare coding variants in TREM2 that increase risk of AD by almost two-fold have been previously identified, making TREM2 the second strongest genetic risk factor for sporadic AD after APOE. Multiple TREM2 risk variants have been identified, but it has been proposed that all TREM2 AD-risk variants cause a partial loss of function. Given the low frequency of these variants, performing separate analysis for each specific variant would not provide enough statistical power. For these reasons, all TREM2 variant carriers were combined in these analyses. Proteomic data was generated from 21, 47, and 131 TREM2 variant carriers in brain, CSF, and plasma, respectively (Table 1). To identify multi-tissue proteomic signatures of individuals carrying AD-risk variants in TREM2, the protein levels of TREM2 variant carriers were compared with both cognitively normal individuals and individuals who were diagnosed with AD dementia, but did not carry any TREM2 or autosomal dominant variant. This is the first time a proteomic profile for TREM2 variant carriers has been generated.

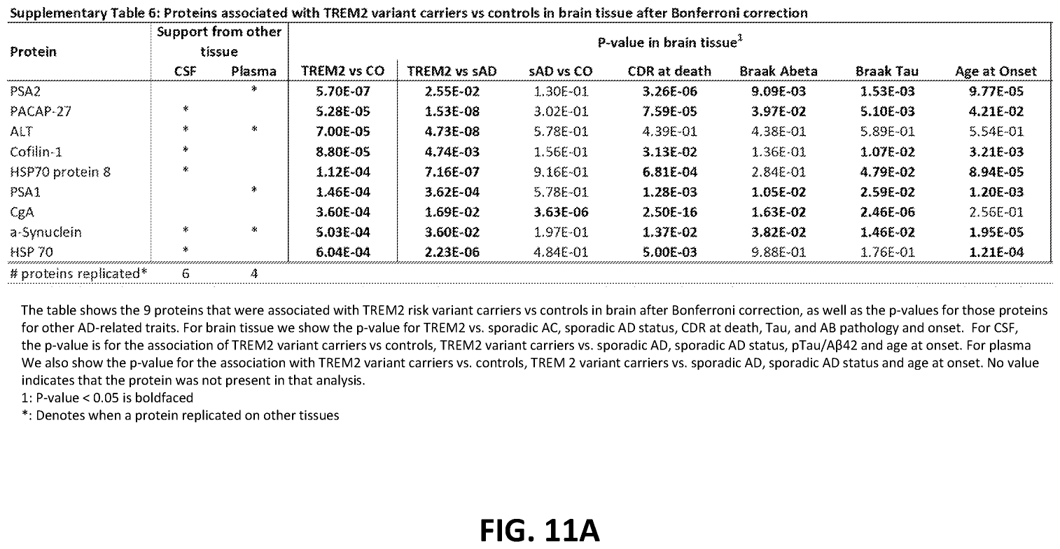

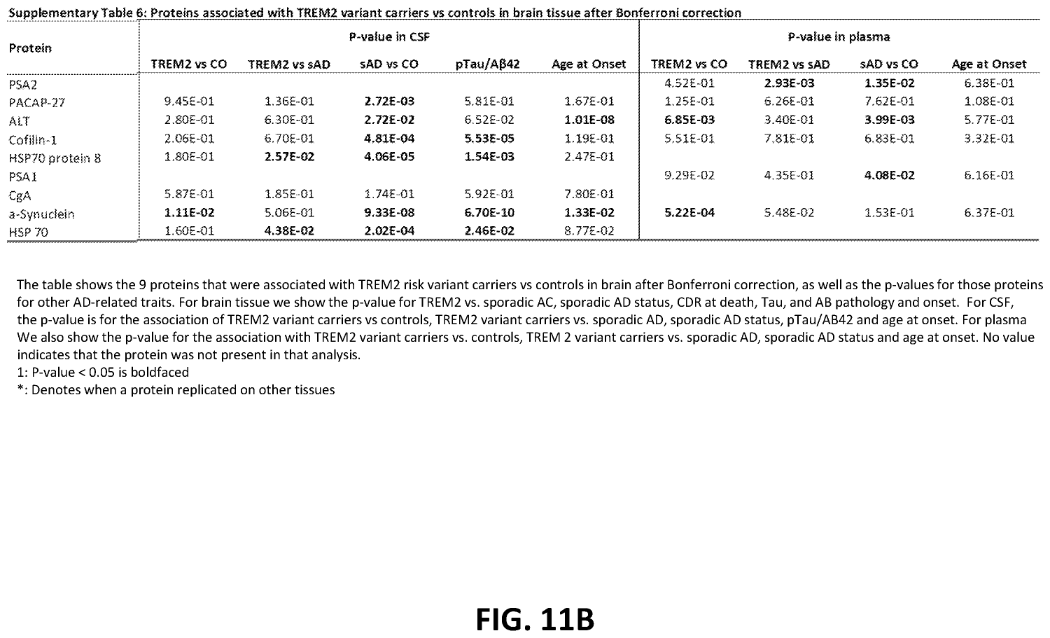

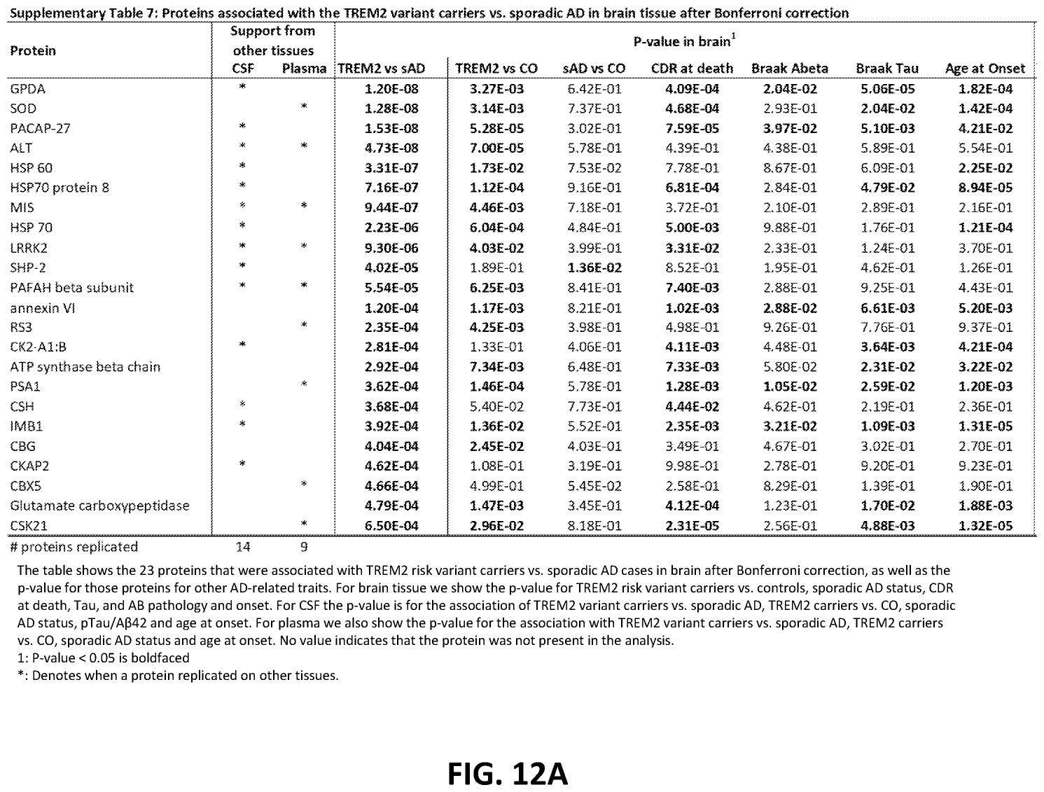

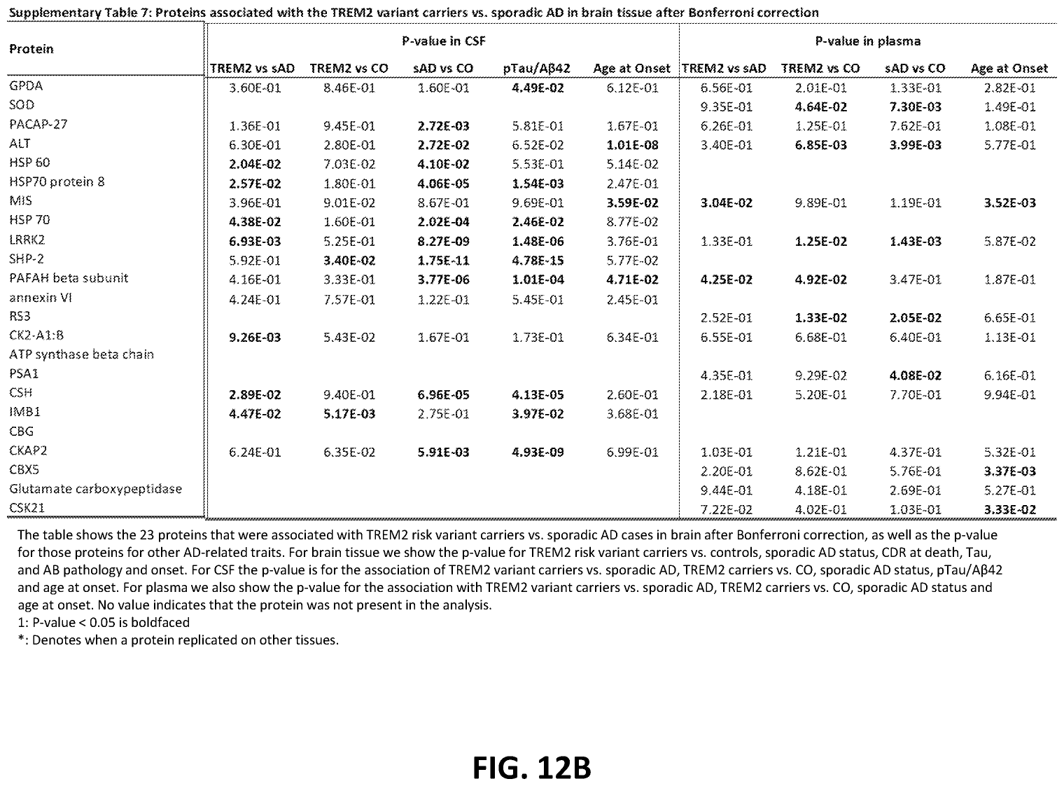

In the brain, 9 proteins (including α-Synuclein) showed differential abundance levels in TREM2 variant carriers compared to cognitively normal individuals at Bonferroni-corrected threshold (FIG. 3A, and Supplementary Table 6 in FIG. 11(A-B)). In addition, 23 proteins (including LRRK2) were associated with AD status after multi-test correction for TREM2 risk variant carriers vs. AD (Supplementary Table 7 in FIG. 12(A-B)). From the genetic data available for the replication datasets, 4 TREM2 variant carriers were found in Mayo, 7 in MSBB, and 8 in ROSMAP. This low number did not provide any statistical power to support a replication analysis. As demonstrated, the multi-tissue study design described herein is a viable alternative approach to identify proteins that would replicate in external datasets, and the data was leveraged to identify those proteins that replicate across tissues. Out of these 27 unique TREM2-associated proteins (combining 9 and 23 proteins), 11 passed QC in both CSF and plasma, and 5 (ALT, α-Synuclein, MIS, LRRK2, and PAFAH beta subunit) replicated in both tissues. This represents a 74-fold enrichment (p=7.53×10⁻⁹) to what would be expected by chance.

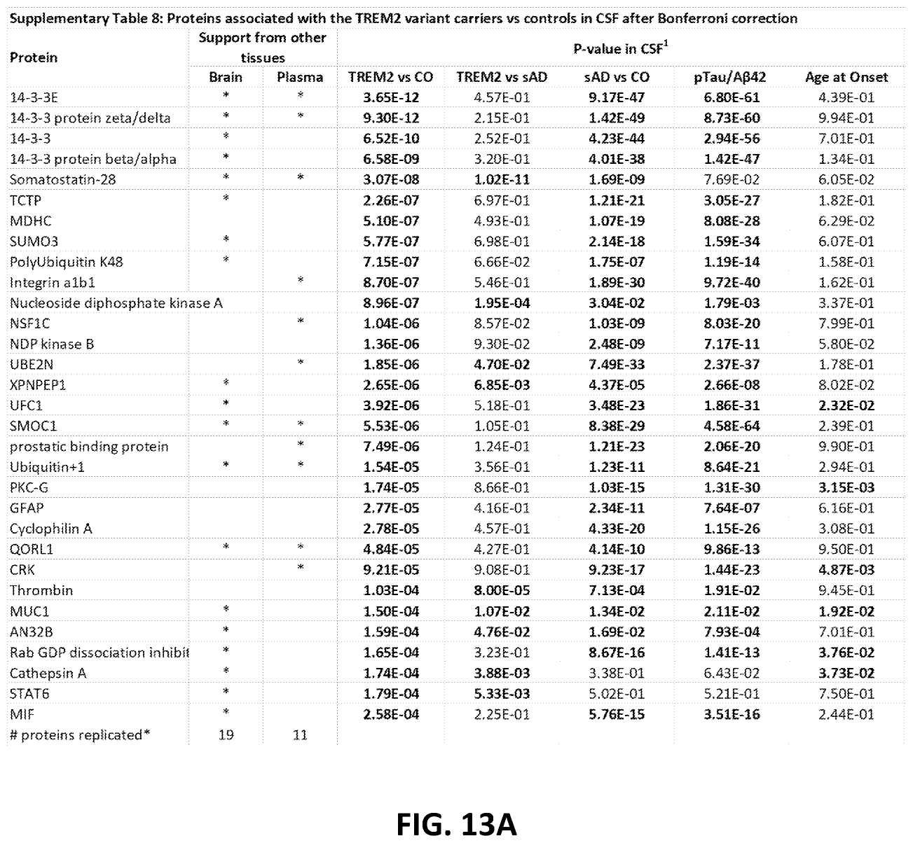

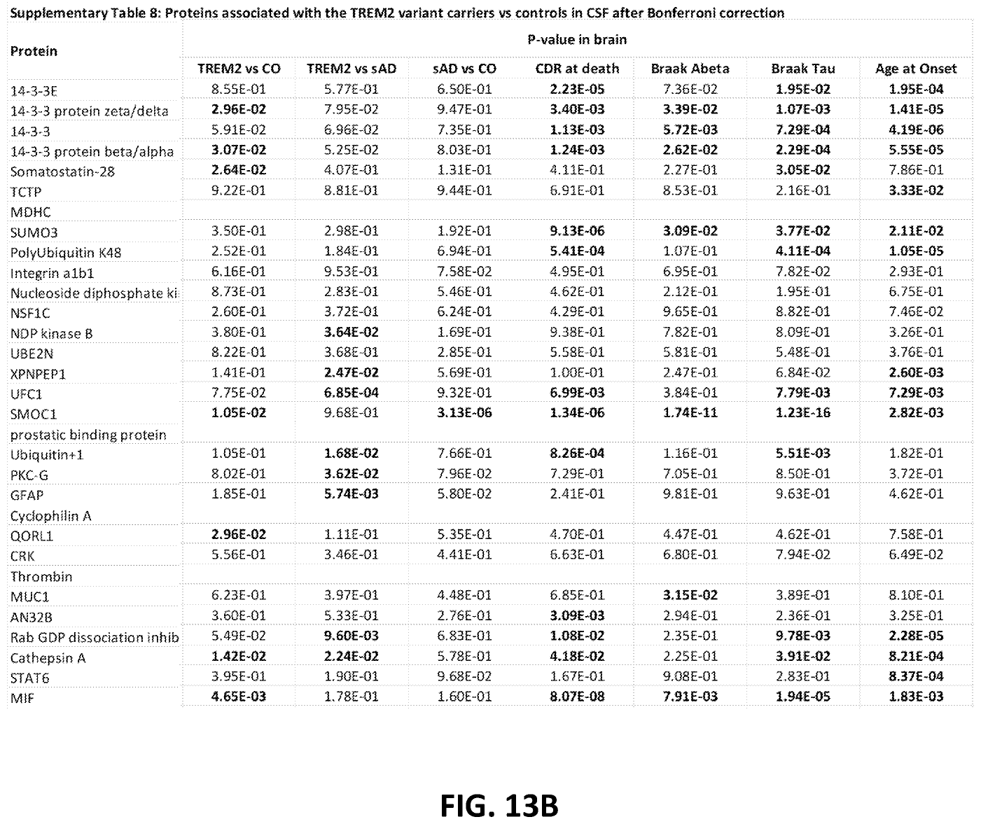

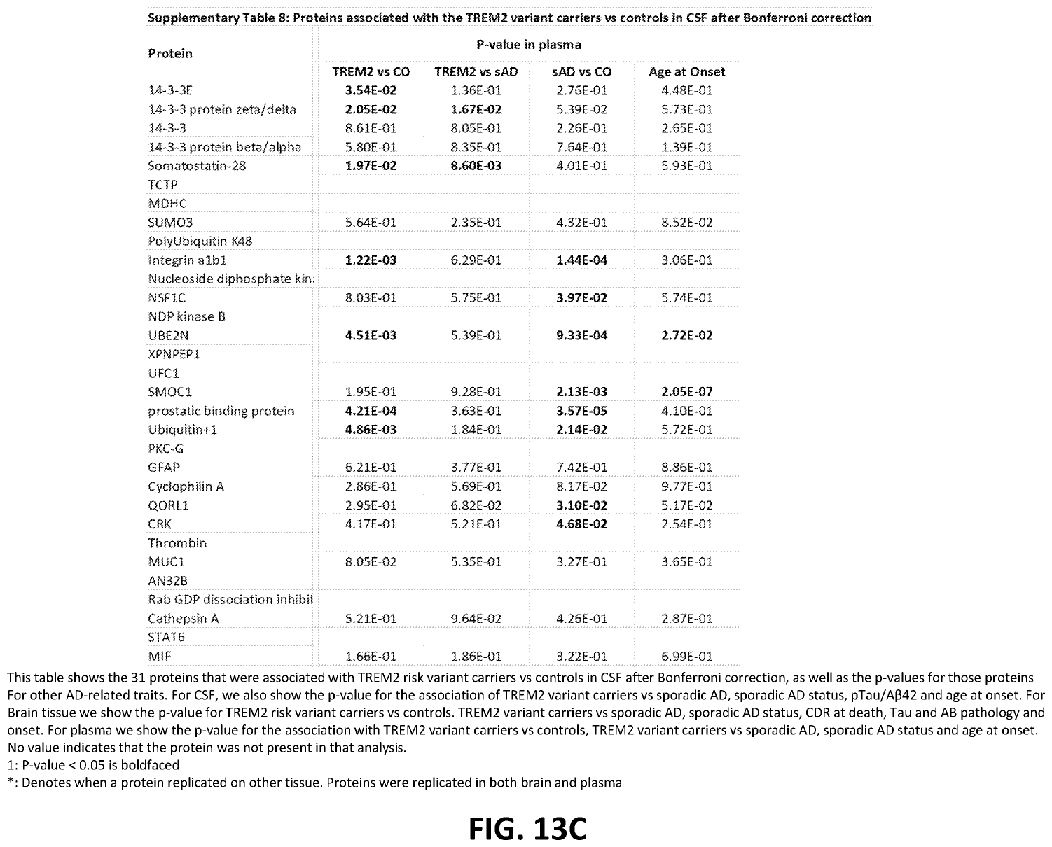

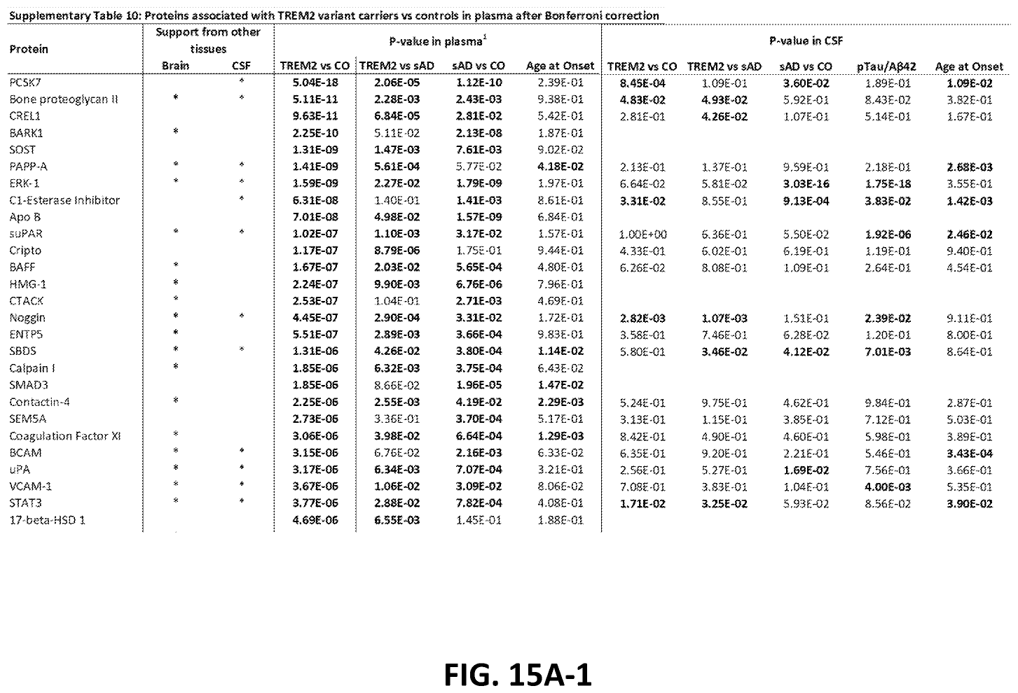

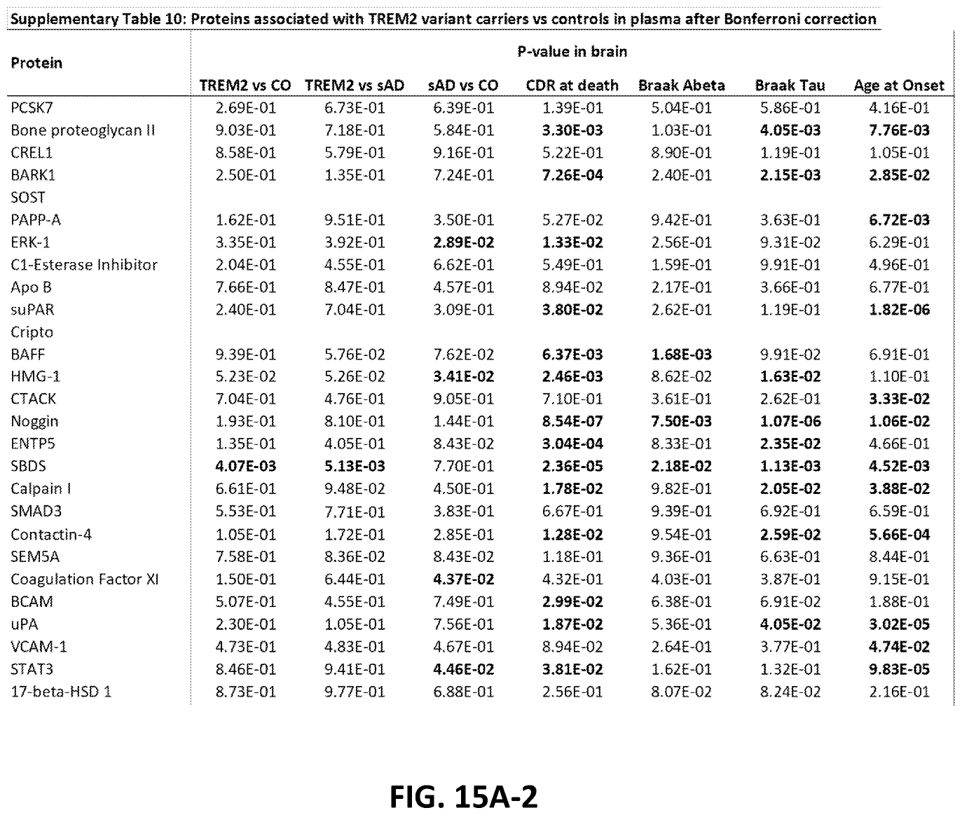

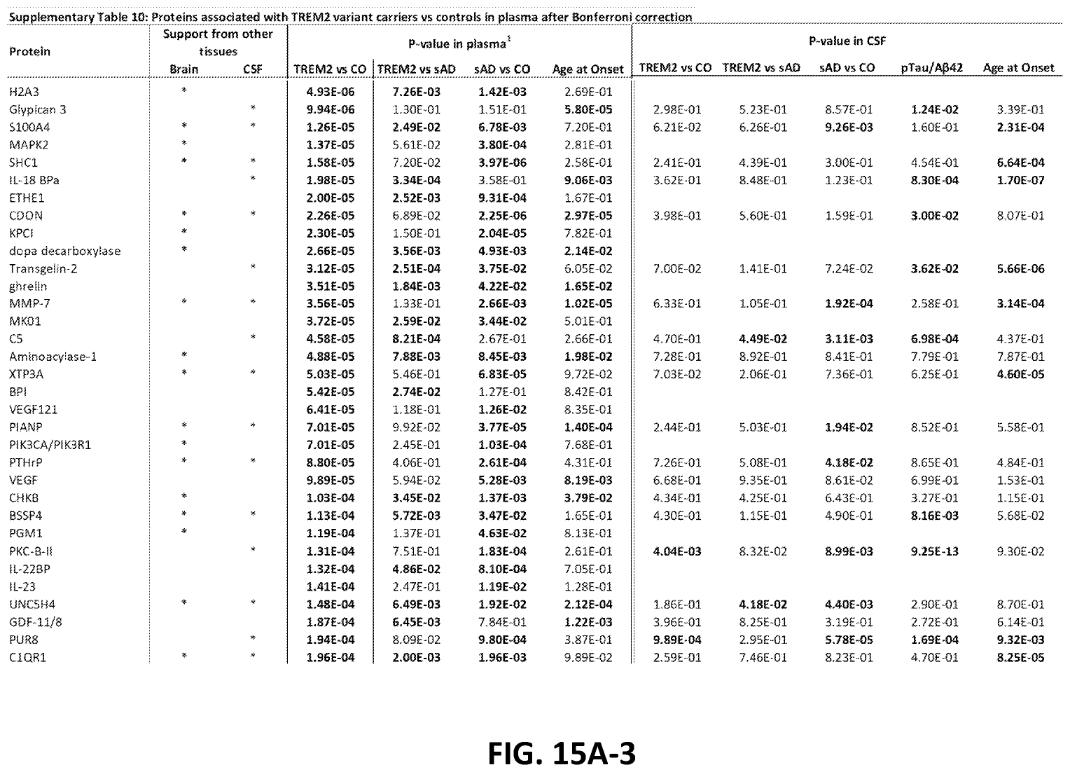

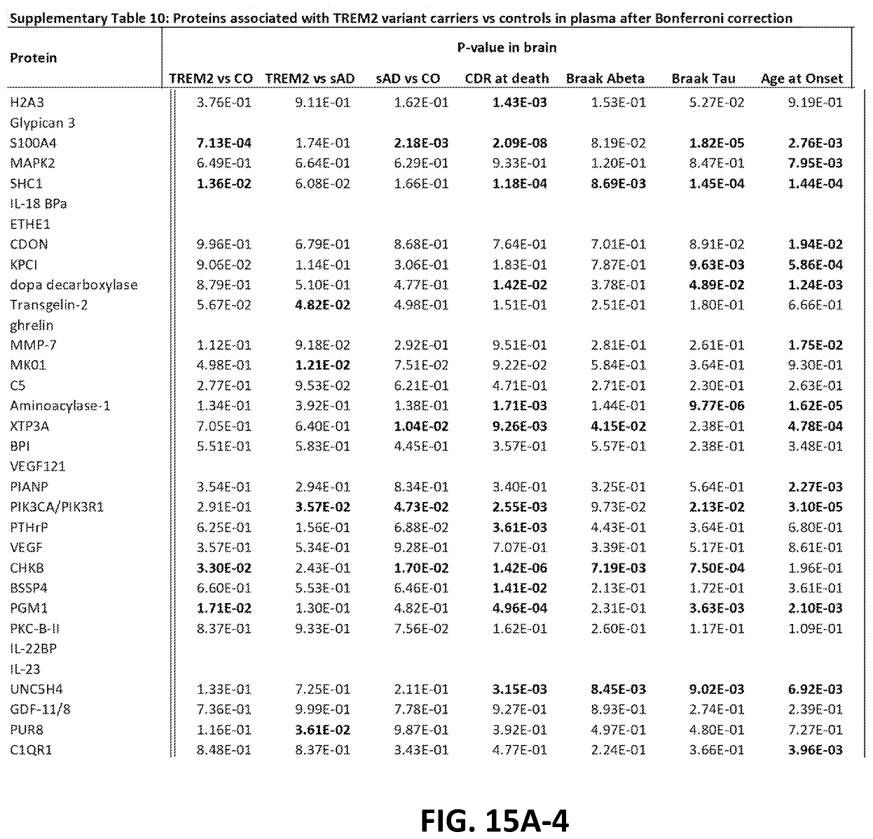

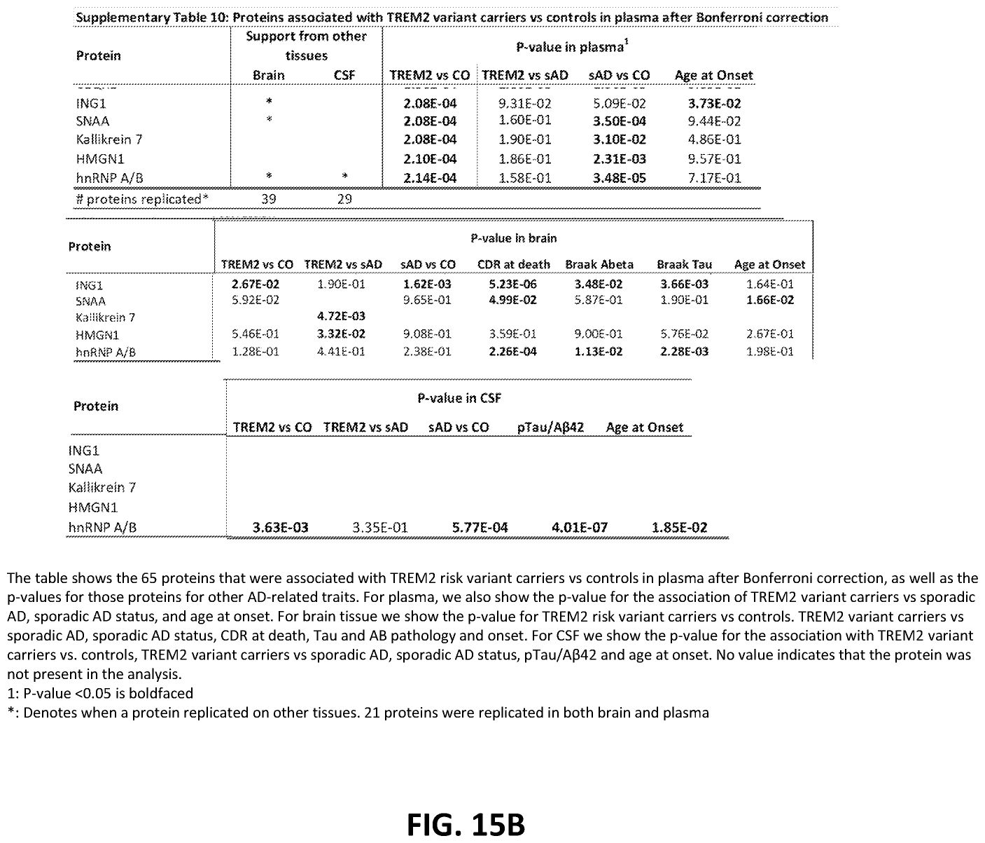

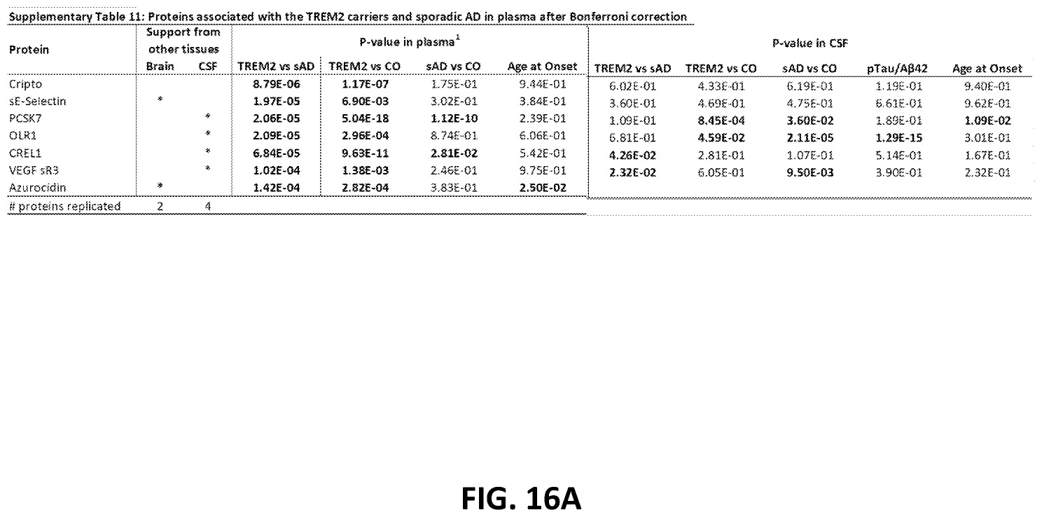

In CSF, these analyses identified a total of 38 unique proteins, among which 31 were associated with TREM2 risk variant carriers vs. cognitively normal individuals and 10 for the TREM2 vs. AD, after multiple test correction (Supplementary Tables 8-9 in FIGS. 13(A-C) and 14(A-B), respectively). Out of these 38 proteins, 20 passed QC in the other tissues, and 7 (14-3-3E, 14-3-3 protein zeta/delta, Somatostatin-28, SMOC1, Ubiquitin+1, QORL1 and calcineurin) replicated across tissues (Supplementary Tables 8-9 in FIGS. 13(A-C) and 14(A-B), respectively). This represents a 73-fold enrichment (p=7.19×10⁻¹²) to what would be expected by chance. In the plasma proteomic data, a total of 69 proteins was identified, among which 65 and 7 showed differential abundance levels in TREM2 variant carriers compared to cognitively normal individuals and to individuals who were diagnosed with AD dementia, respectively (Supplementary Tables 10-11 in FIGS. 15(A-B) and 16(A-B), respectively). Among the 41 proteins that passed QC in the brain and CSF, 21 proteins (including bone proteoglycan II, PAPP-A, ERK-1, suPAR and VCAM-1) replicated, which represents a 122-fold enrichment (p=5.47×10⁻³) to what would be expected by chance.

Autosomal Dominant AD Status

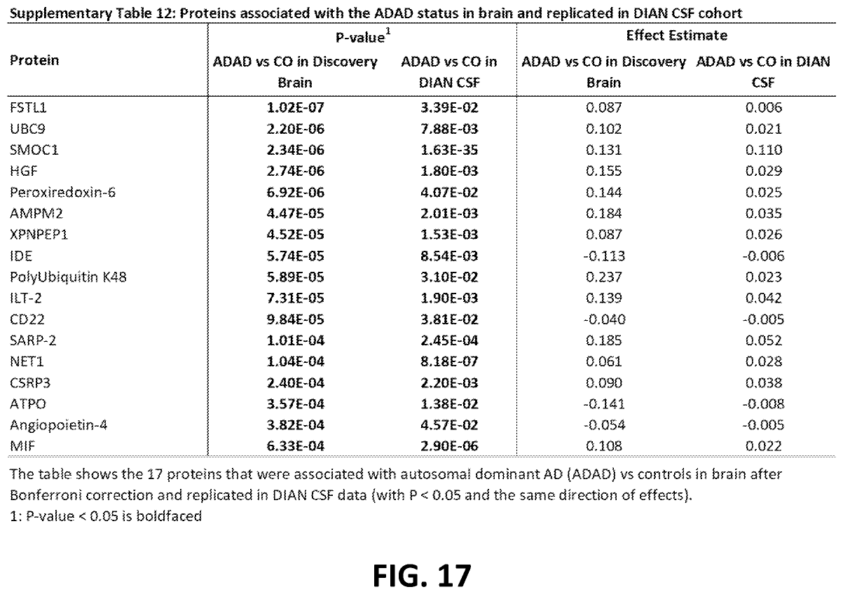

Although most AD cases are considered sporadic and manifest after the age of 65, around 1-3% of AD cases show an autosomal dominant (ADAD) inheritance pattern, often with onset before age 65. Pathogenic variants in APP, PSEN1 and PSEN2 have been identified as the cause of ADAD. Proteomic data was generated from the parietal cortex of 24 ADAD gene variant carriers (19 individuals with PSEN1, 1 with PSEN2, and 4 with APP variants) recruited from the DIAN and the Knight ADRC studies. 109 proteins were identified with differential abundance in ADAD mutation carriers compared to cognitively normal individuals with no significant brain pathology, at Bonferroni corrected threshold. In order to validate these findings, whether these 109 proteins were also associated with ADAD status in CSF from 289 carriers and 184 non-carriers from the DIAN study was analyzed. Due to the limited amount of CSF samples for these subjects, proteomic discovery in sporadic AD or TREM2 variant carriers was not performed. From those 109 proteins identified in brain, 106 passed QC in CSF proteomic data and 17 were associated with ADAD in CSF and in the same direction (FIG. 4(A-B), and Supplementary Table 12 in FIG. 17), which represents a 6.4-fold enrichment (p=1.36×10⁻⁹) to what would be expected by chance.

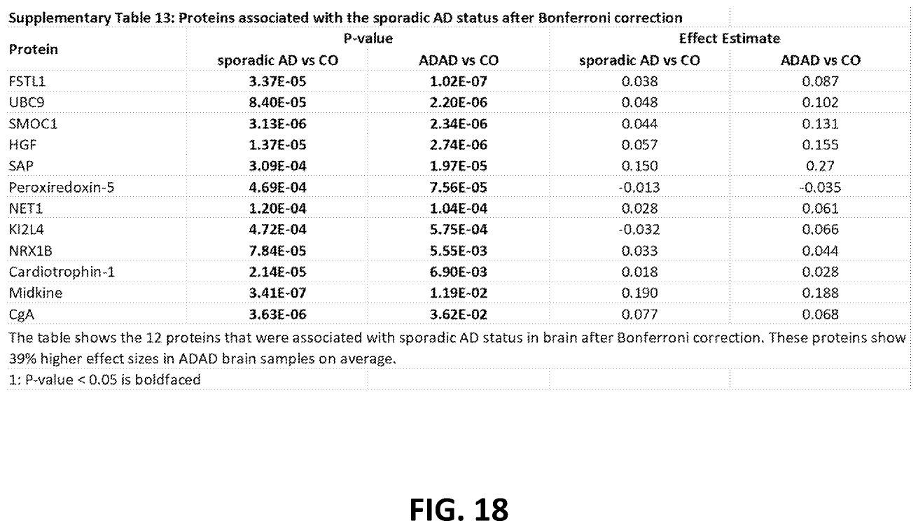

As discussed above, 12 proteins were identified to be associated with sporadic AD status in brain tissue (Supplementary Table 2 in FIG. 7(A-C)). Whether the proteins associated with sporadic AD status showed similar differential abundance in ADAD mutation carriers was also tested. It was found that most of the proteins associated with sporadic AD brains displayed even stronger effect size when comparing ADAD mutation carriers to controls (Supplementary Table 13 in FIG. 18). The proteins associated with sporadic AD status showed 39% higher effect sizes in ADAD brain samples on average (P=3.8×10⁻⁵; FIGS. 4A and 4B). For example, SMOC1 showed a significant association AD vs. control (Effect=0.04: P=3.1×10⁻⁶) but also for ADAD vs. CO (Effect=0.13; P=2.3×10⁻⁶). As presented earlier, SMOC1 has also been found to be associated in sporadic AD status in both CSF (P=8.4×10⁻²⁹) and plasma (P=0.002), suggesting that it could be used to create a new prediction model for AD, independent of Aβ and tau.

Tissue-Specific Prediction Models

The analyses identified tissue-specific proteomic signatures for sporadic AD and TREM2 risk variant carriers. Herein, the proteins that replicated in external datasets (for AD status) or across tissues (for TREM2 variant carriers and ADAD) were used to create prediction models. To assess the specificity and selectivity of these prediction models, receiver operator characteristic (ROC) curve and area under the curve (AUC) were computed using the R package pROC. Age at measurement and sex were included as covariates. Analysis was also performed by adding APOE ε4 status as a covariate. In sporadic AD cases, these prediction models were examined for both the discovery and replication datasets.

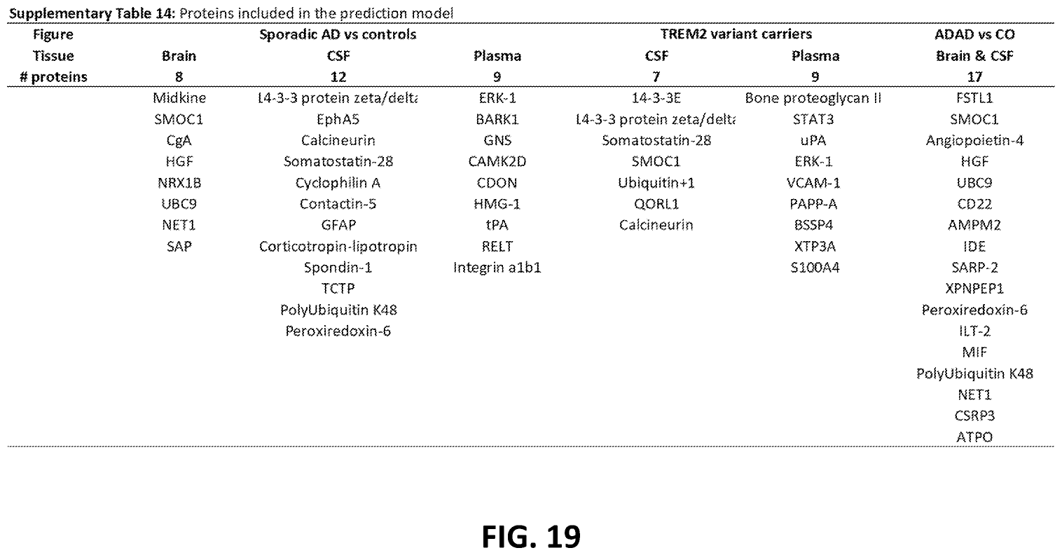

In brain tissue, the prediction model based on the 8 proteins that replicated in the analysis (Supplementary Table 1 in FIG. 6(A-D), and Supplementary Table 14 in FIG. 19) led to an AUC of 0.84 in the discovery and an AUC of 0.99 in the replication cohort (FIG. 2B). In CSF, 39 proteins were found to be associated with AD status that replicated in external datasets (Supplementary Table 3 in FIG. 8A (8A-1 through 8A-4), 8B (8B-1 through 8B-4), and 8C). A prediction model including these proteins led to an AUC of 0.90 in the replication and of 0.89 in the discovery cohort (FIG. 2B). As the number of proteins is too large to generate a prediction model that could be translated to the clinic, the stepwise model selection was performed to identify the minimum set of proteins that capture the same information as the 39 identified in the study. A panel of 12 proteins was found that provided accuracy in distinguishing clinically defined AD patients from controls almost as high as all 39 proteins and led to an AUC of 0.88 in the discovery and 0.999 in replication data. The prediction model was compared to CSF p-tau/Aβ42, known and validated biomarkers. In the dataset the CSF p-tau/Aβ42 ratio led to an AUC of 0.81, which is significantly lower than the prediction model (P=2.4×10-6). Using the same approach for plasma, the 9 proteins identified and replicated in an external dataset (Supplementary Table 4 in FIG. 9(A-B), and Supplementary Table 14 in FIG. 19) led to an AUC of 0.79 in both discovery and replication datasets, which was not statistically different from the AUC with CSF p-tau/Aβ42 ratio (AUC=0.82; P>0.05). The prediction model based on each externally replicated protein is similar between the discovery and replication data.

Prediction models were also created that could distinguish TREM2 variant carriers from non-carriers in both sporadic AD cases and controls. Therefore, the proteins that were differentially abundant between TREM2 risk variant carriers when compared not only to AD cases but also to controls were included. Due to a lack of external datasets, only those proteins that replicated across tissues were included, as explained above. In CSF, the prediction model that included 7 proteins (Supplementary Tables 8-9 in FIGS. 13(A-C) and 14(A-B), respectively) resulted in an AUC of 0.79 when comparing TREM2 risk variant carriers to controls. The same proteins showed an AUC of 0.84 for TREM2 risk variant carriers compared to AD cases (FIG. 3B). CSF p-tau/Aβ42 levels have been shown to be a very good biomarker to distinguish AD cases vs controls, but no previous studies examined how CSF p-tau/Aβ42 ratio provides prediction for TREM2 variant carriers. In this study, CSF p-tau/Aβ42 showed an AUC of 0.74 for TREM2 variant carriers vs AD cases and AUC of 0.53 for TREM2 risk variant carriers vs cognitively normal individuals. Both AUC values are significantly lower than those from the TREM2-associated prediction model with 7 proteins (P<1.6×10⁻⁵; FIG. 3B).

In plasma, the 21 proteins included in the model (Supplementary Tables 10-11 in FIGS. 15(A-C) and 16(A-B), respectively) led to an AUC of 0.93 in differentiating TREM2 risk variant carriers from controls, while the CSF p-tau/Aβ42 ratio led to a significantly lower AUC of 0.69 (P=1.1×10⁻³). Similarly, in differentiating TREM2 risk carriers from other AD cases, the same 21 proteins led to an AUC of 0.90, which is significantly higher (P=1.5×10⁻⁴) than the AUC with the CSF p-tau/Aβ42 ratio (AUC=0.63). As the number of proteins is large, a stepwise model selection was performed and a subset of 9 proteins was found that provided AUCs of 0.89 and 0.88 to discriminate TREM2 variant carriers from cognitively normal individuals and from individuals with AD dementia, respectively (FIG. 3B). The prediction models including age, sex and APOE ε4 status as covariates provided similar performance.

The 17 proteins that were found to be associated with ADAD status and in the same direction in brain and CSF (Supplementary Table 12 in FIG. 17) were also leveraged to create potential prediction models for distinguishing ADAD mutation carriers from non-carriers. In brain data, the model with these 17 proteins provided an AUC of 1, which is significantly higher than the model based on age alone (AUC=0.76; P=9.9×10⁻³). In CSF data, the same 17 proteins provided a higher AUC value than the model with age alone (AUC=0.87 vs 0.53, P<2.2×10⁻¹⁶; FIGS. 4A and 4B).

Pathway Enrichment

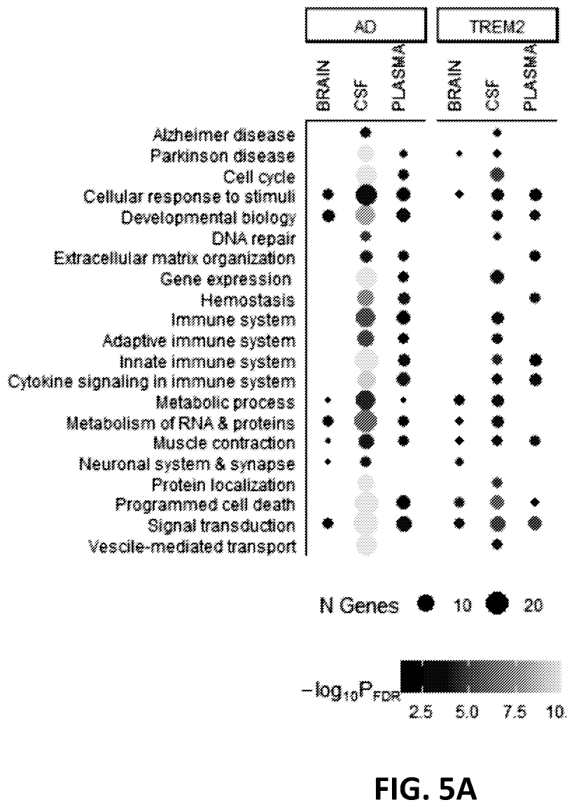

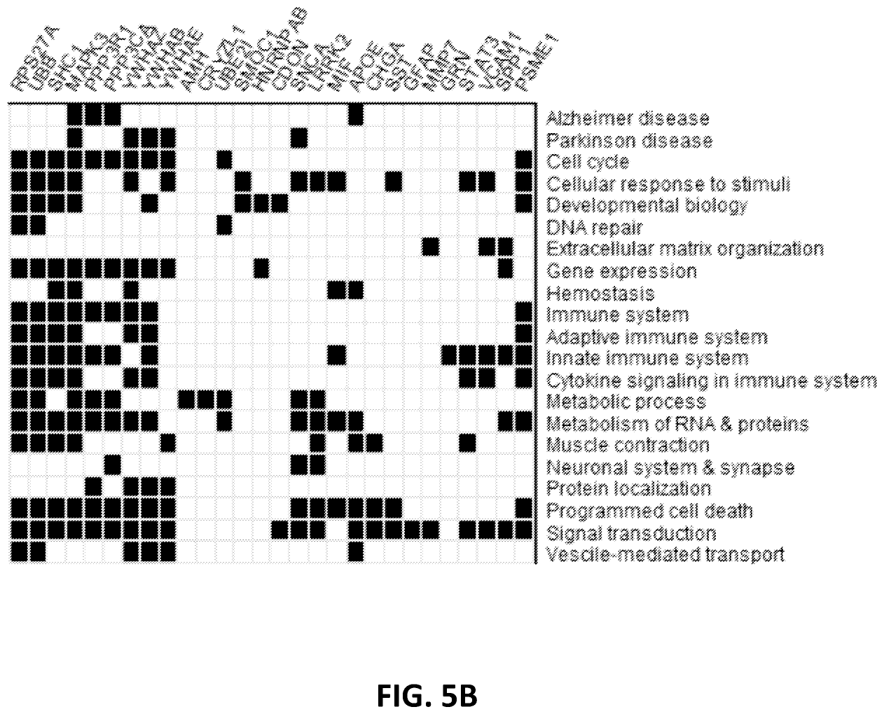

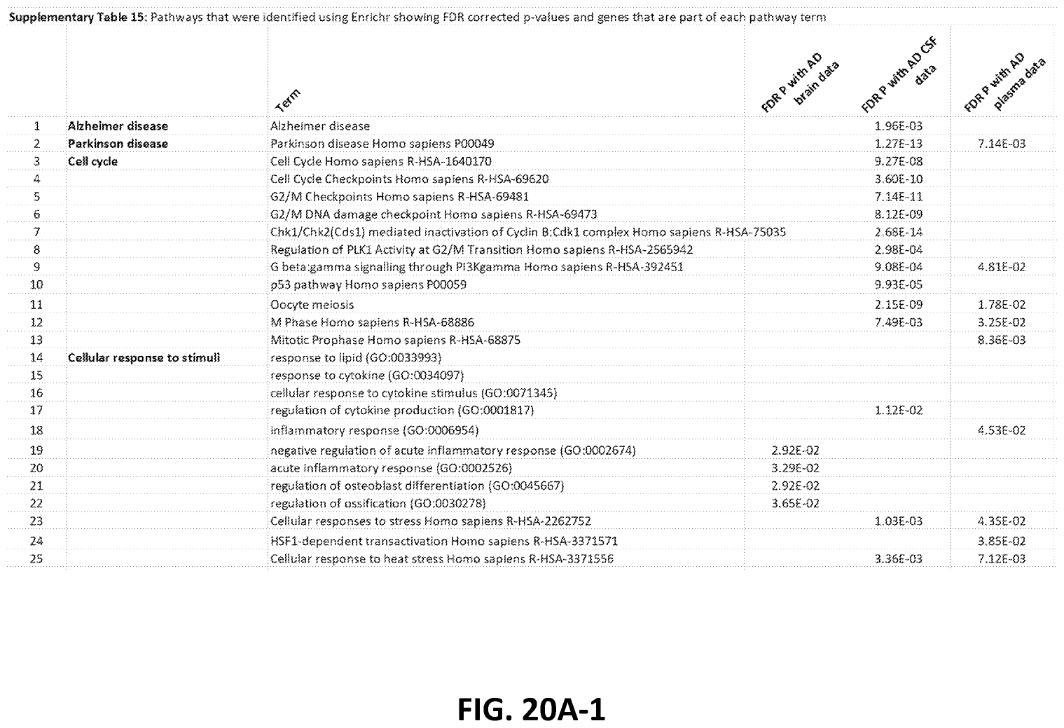

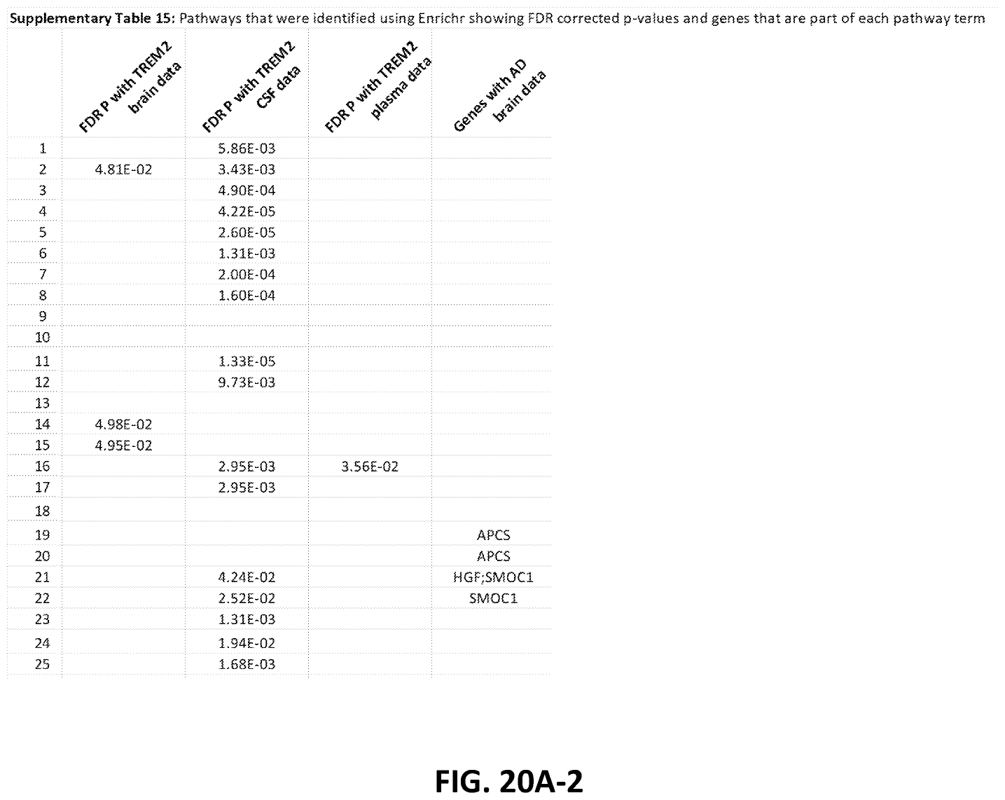

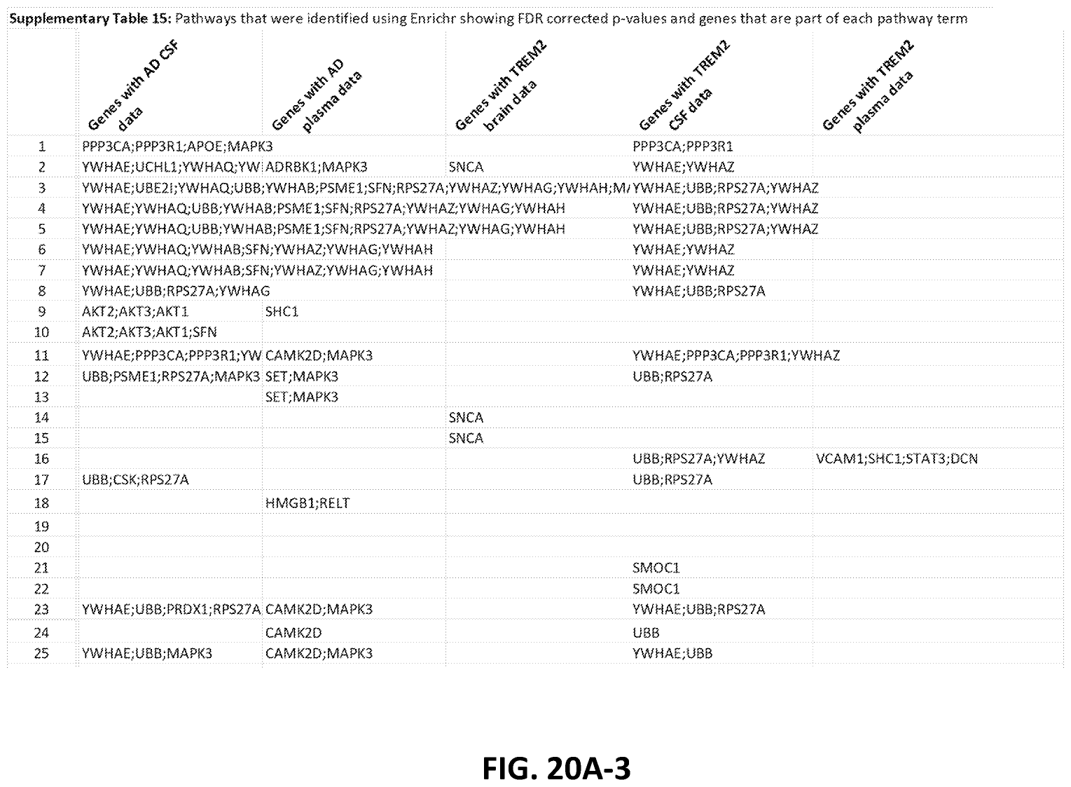

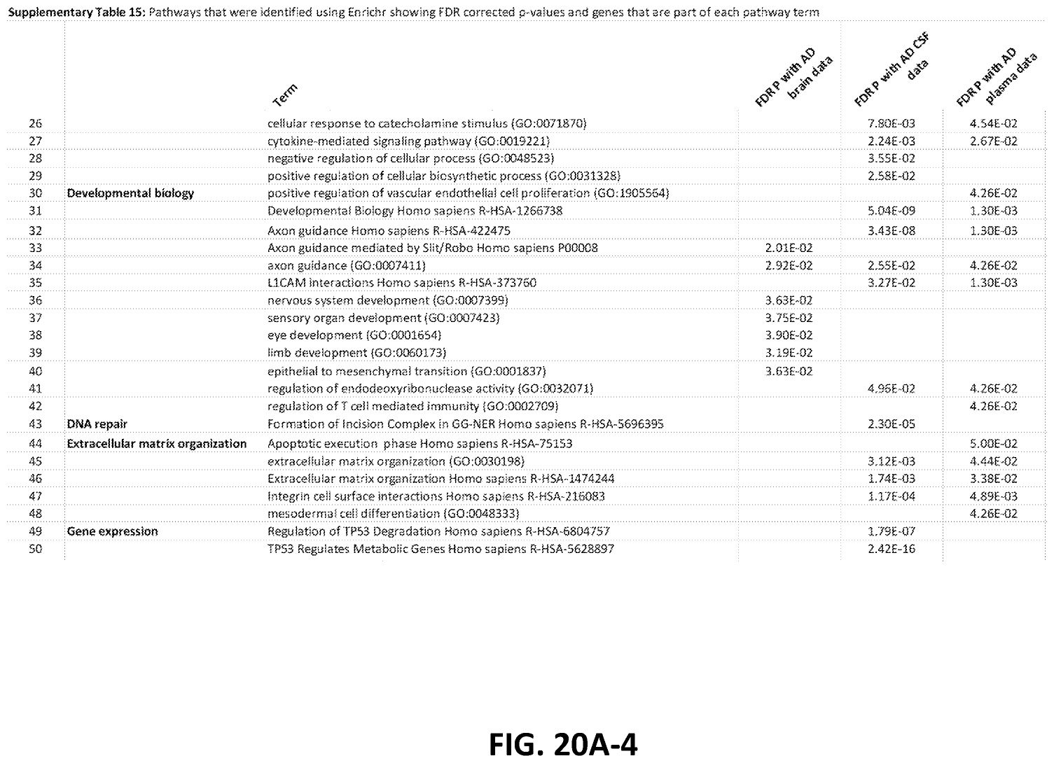

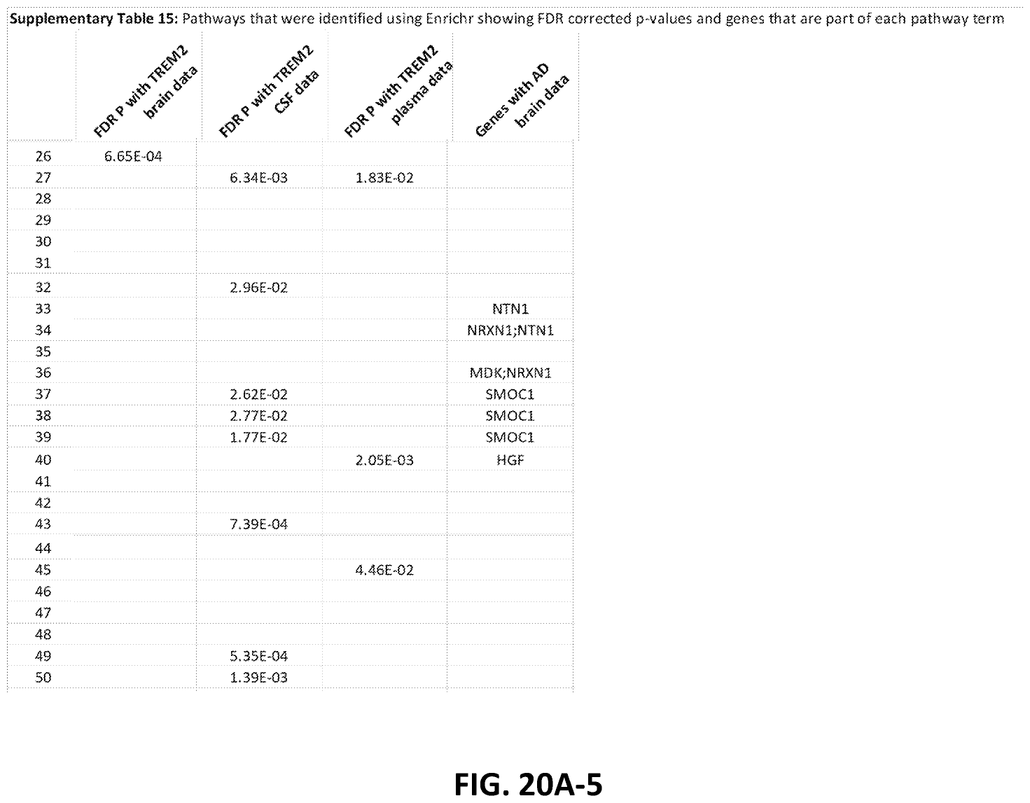

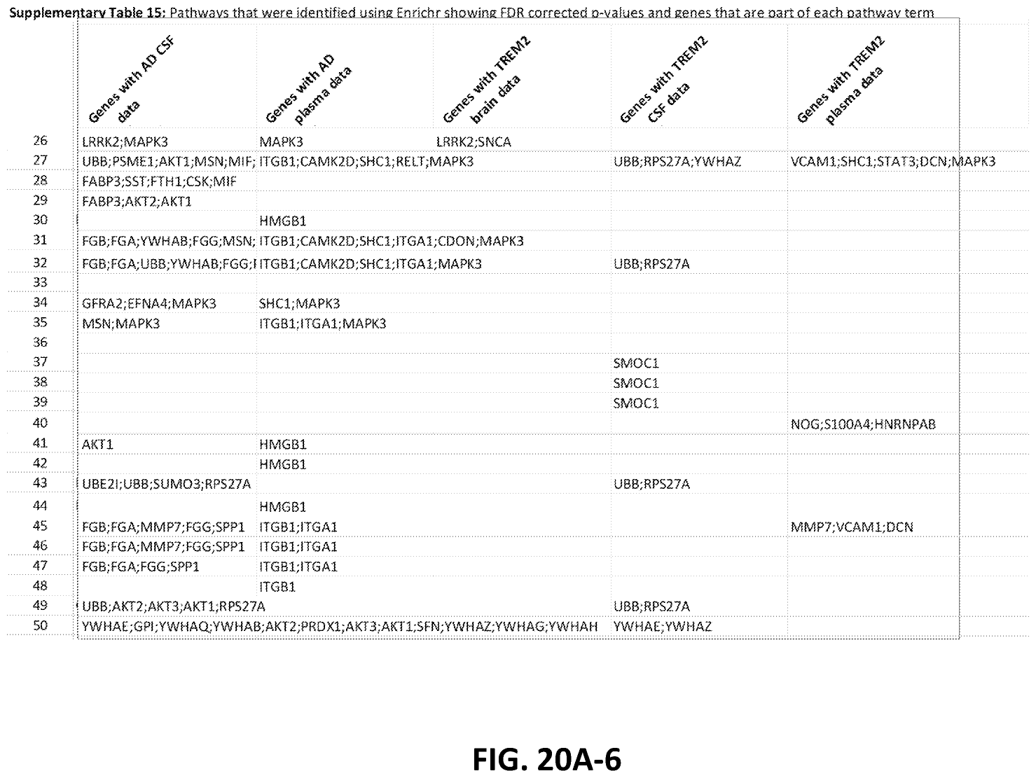

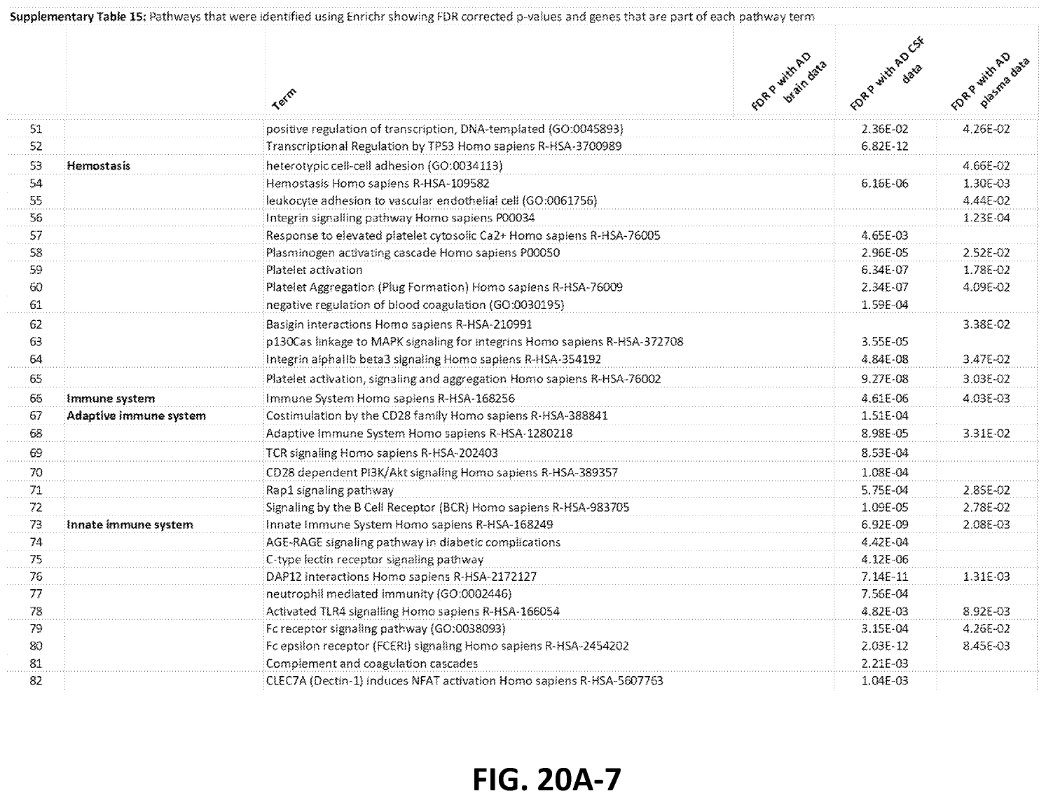

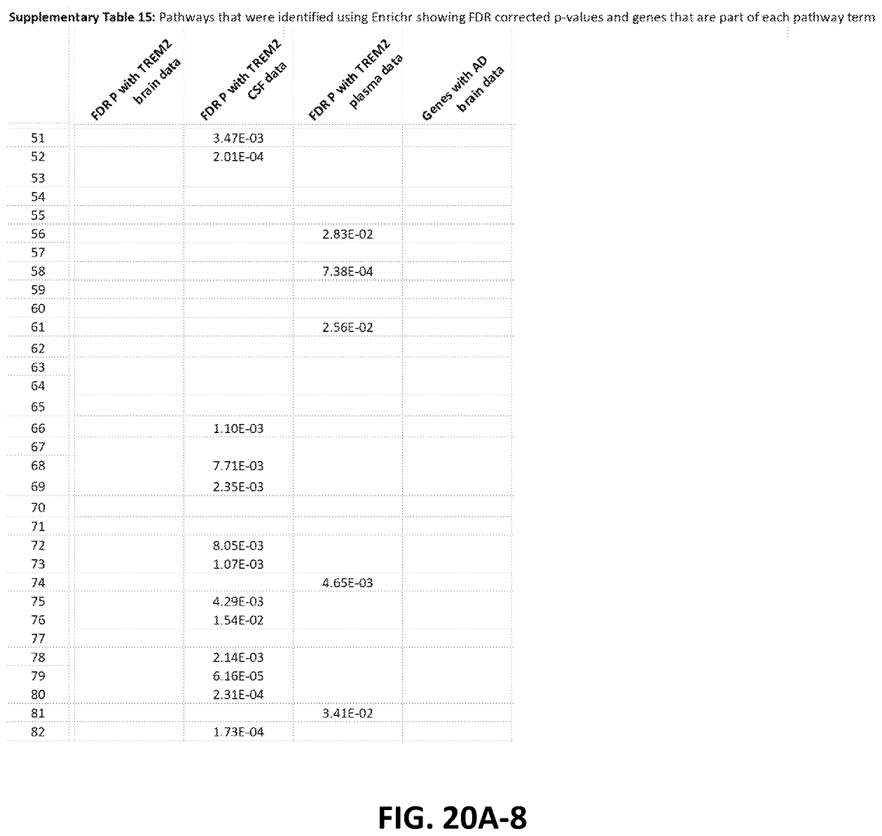

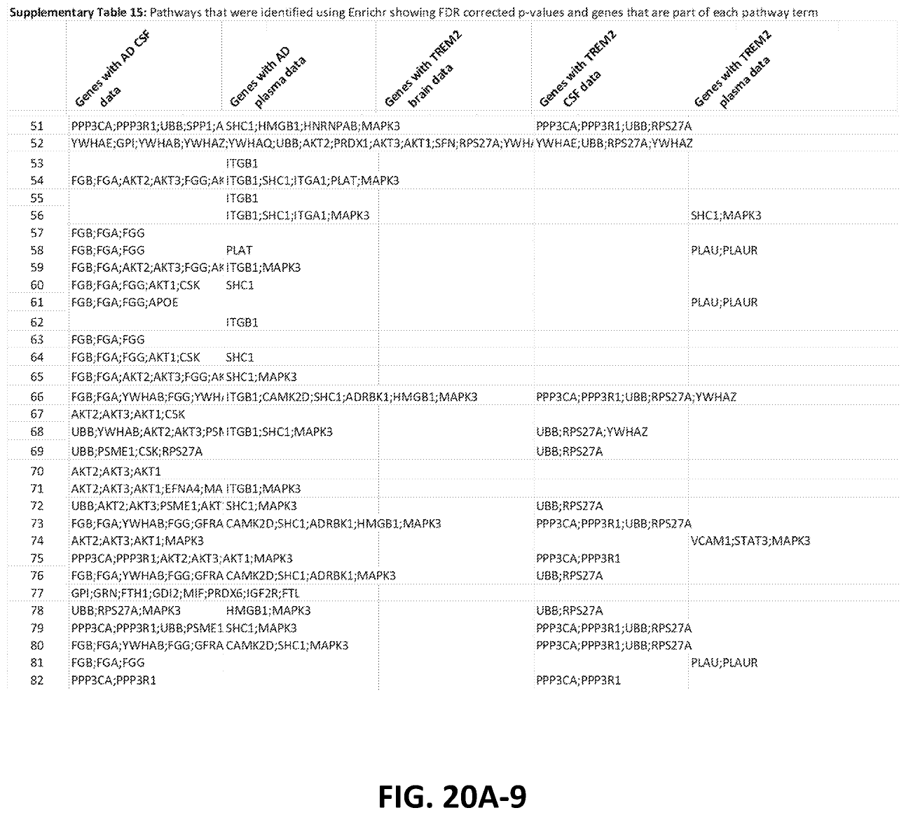

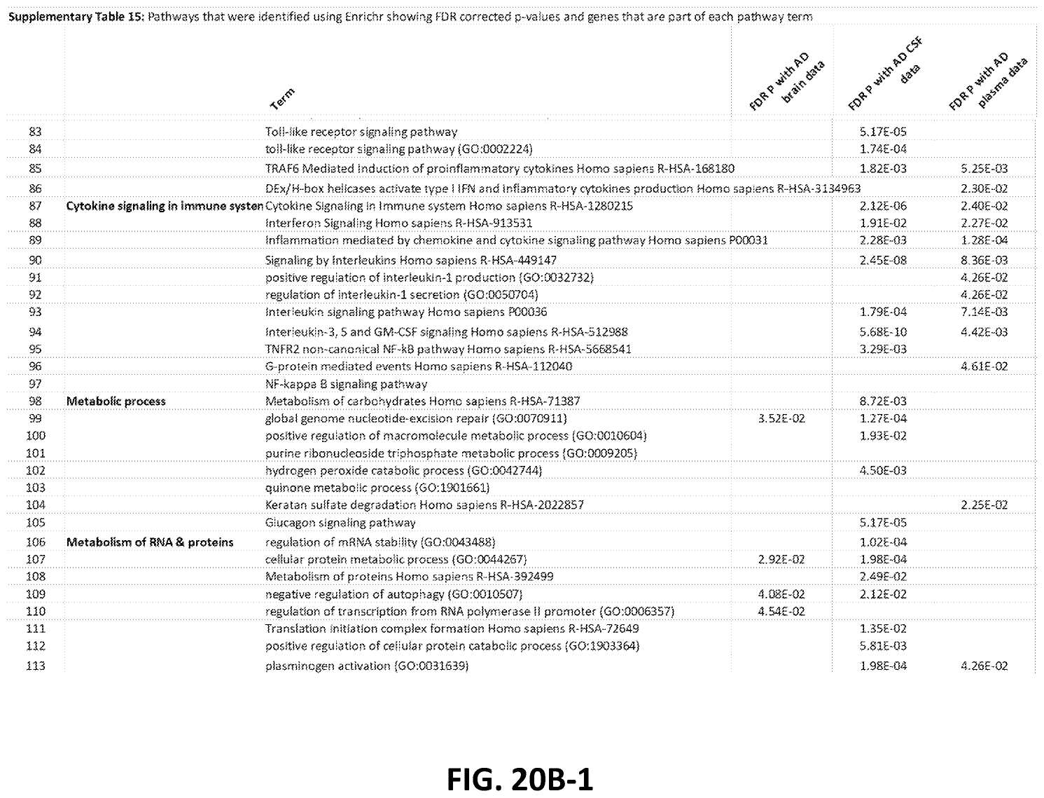

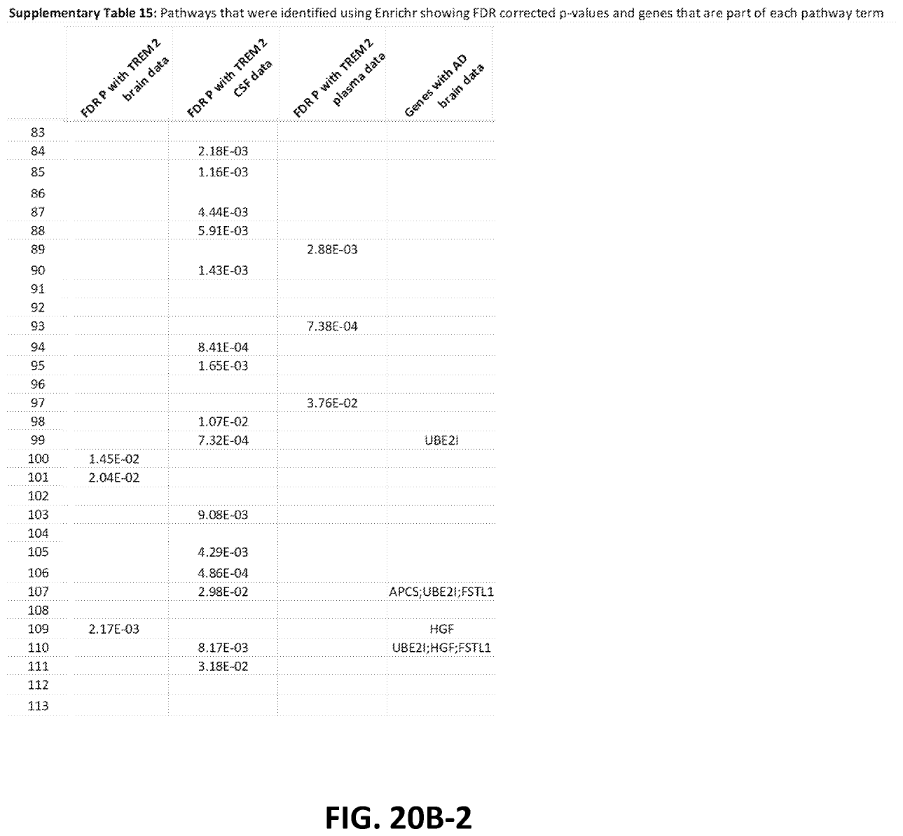

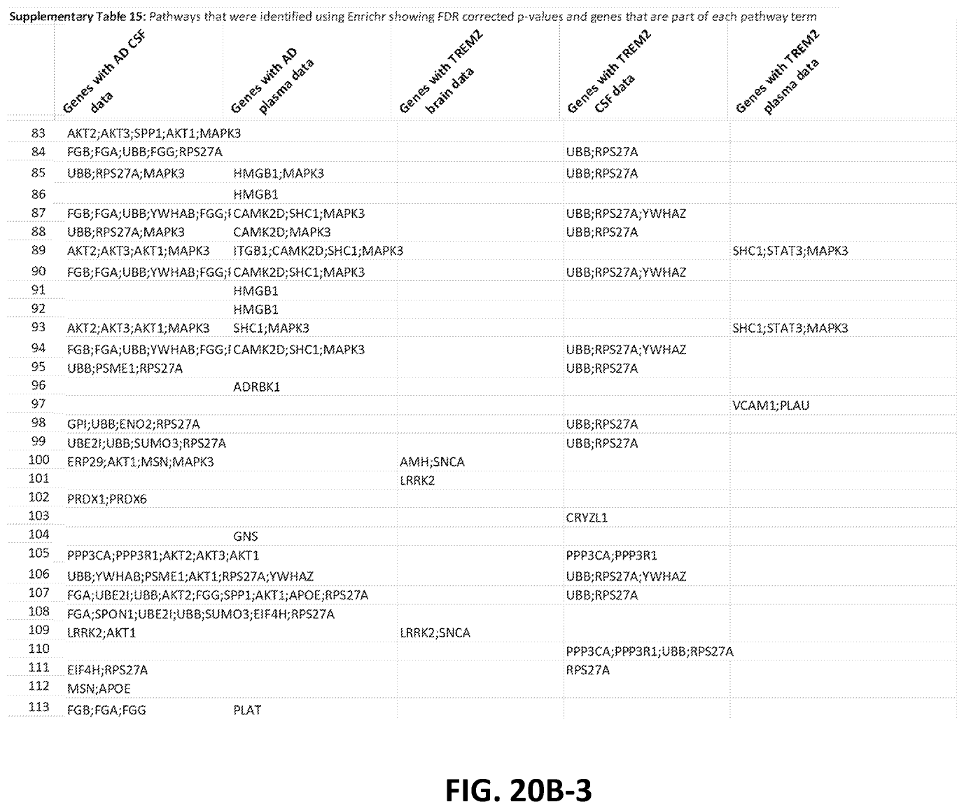

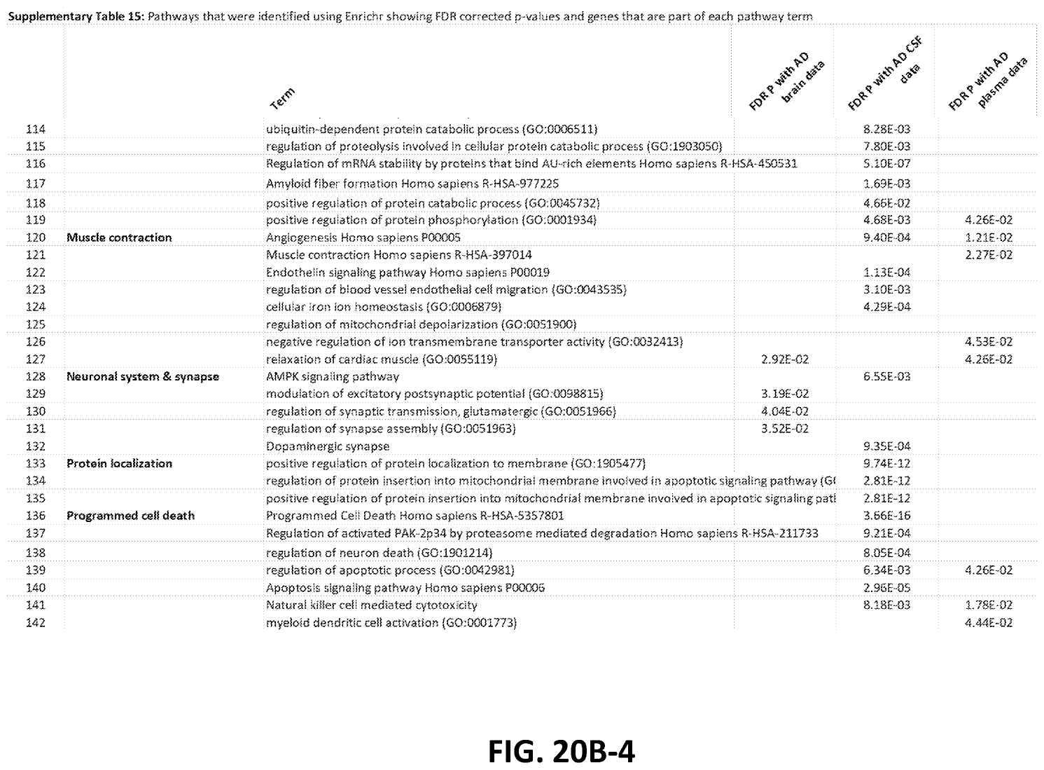

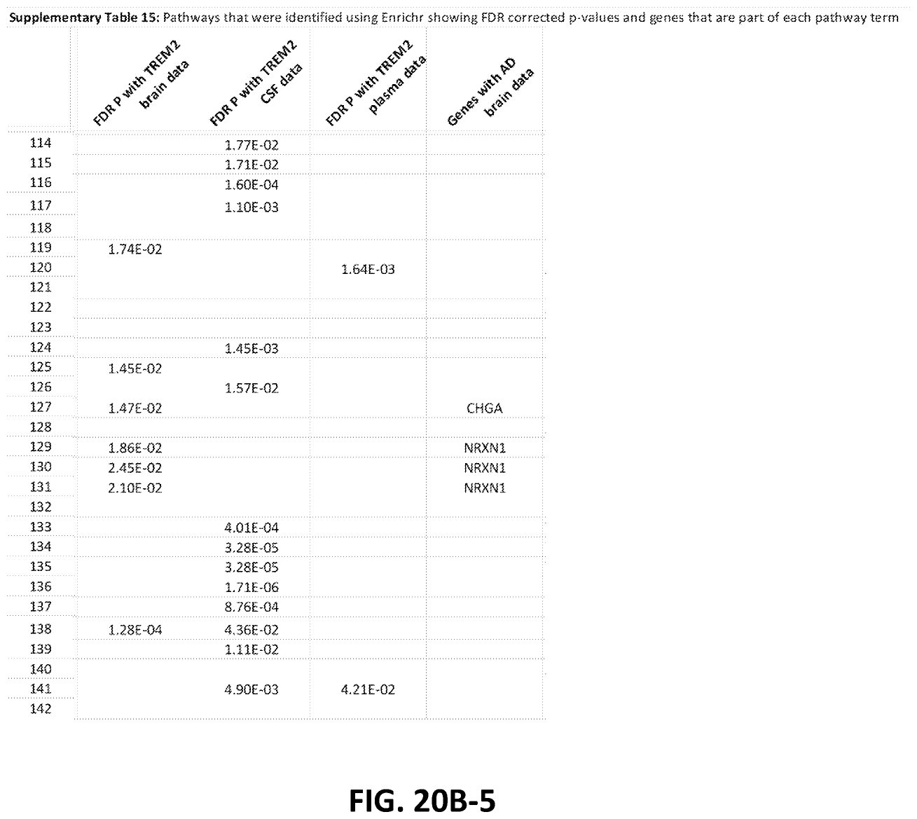

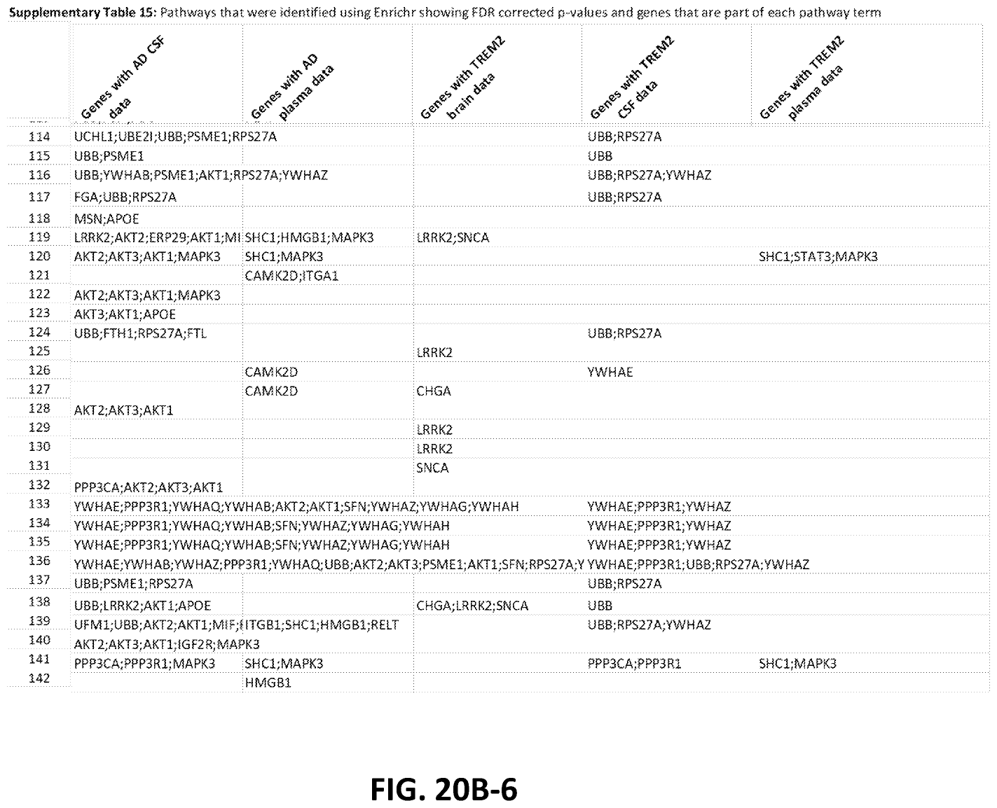

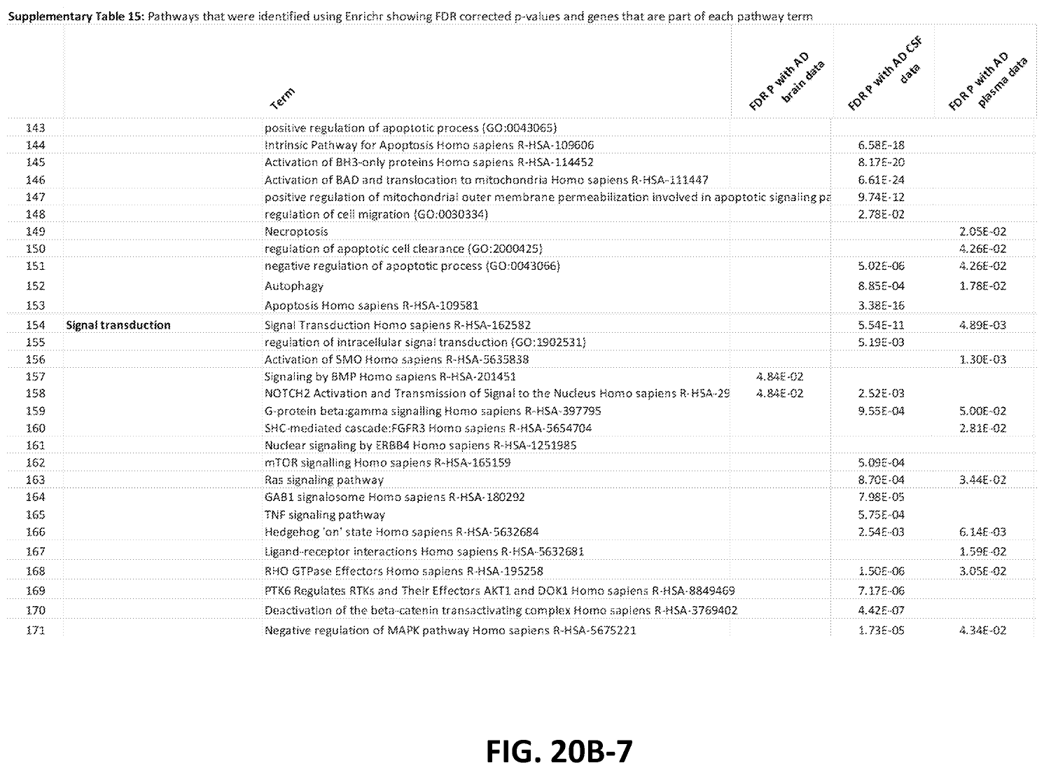

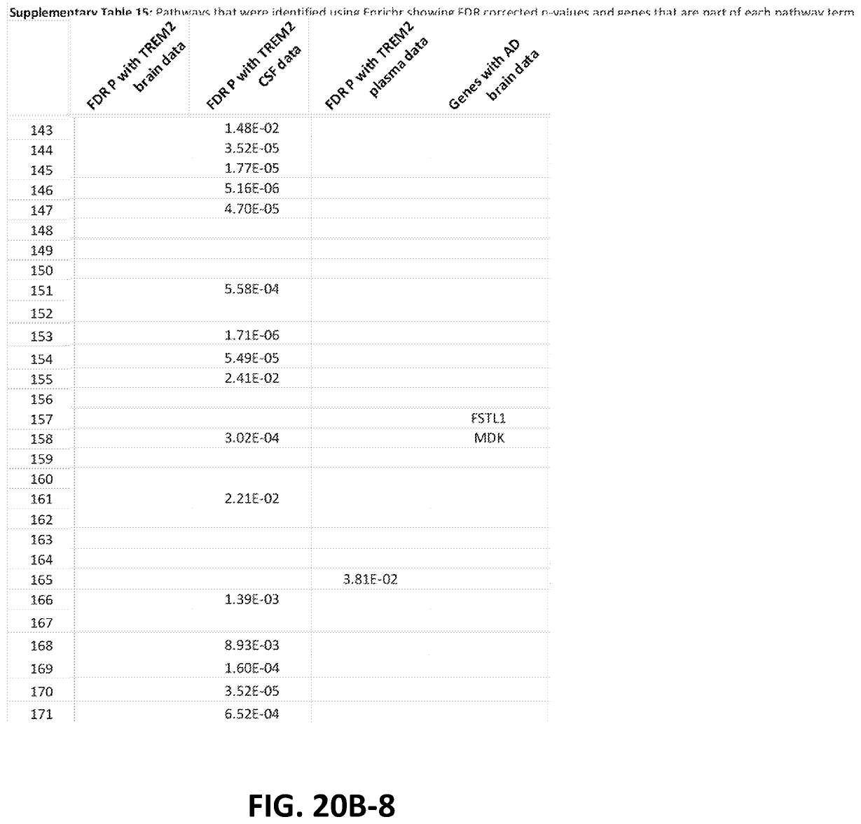

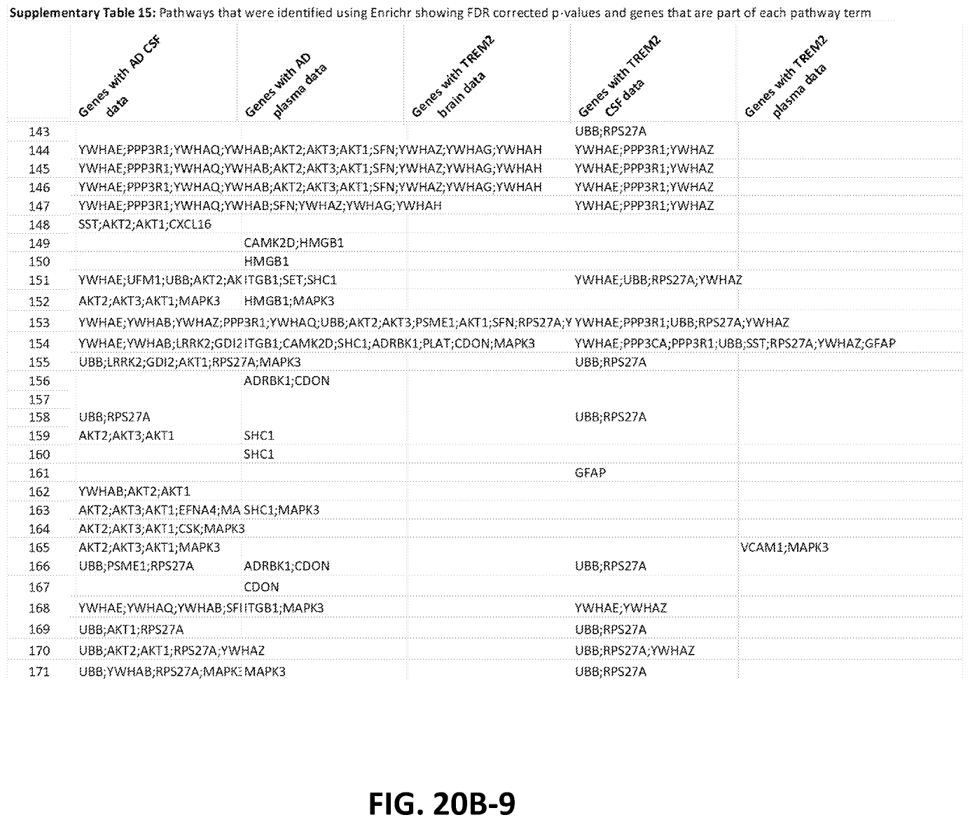

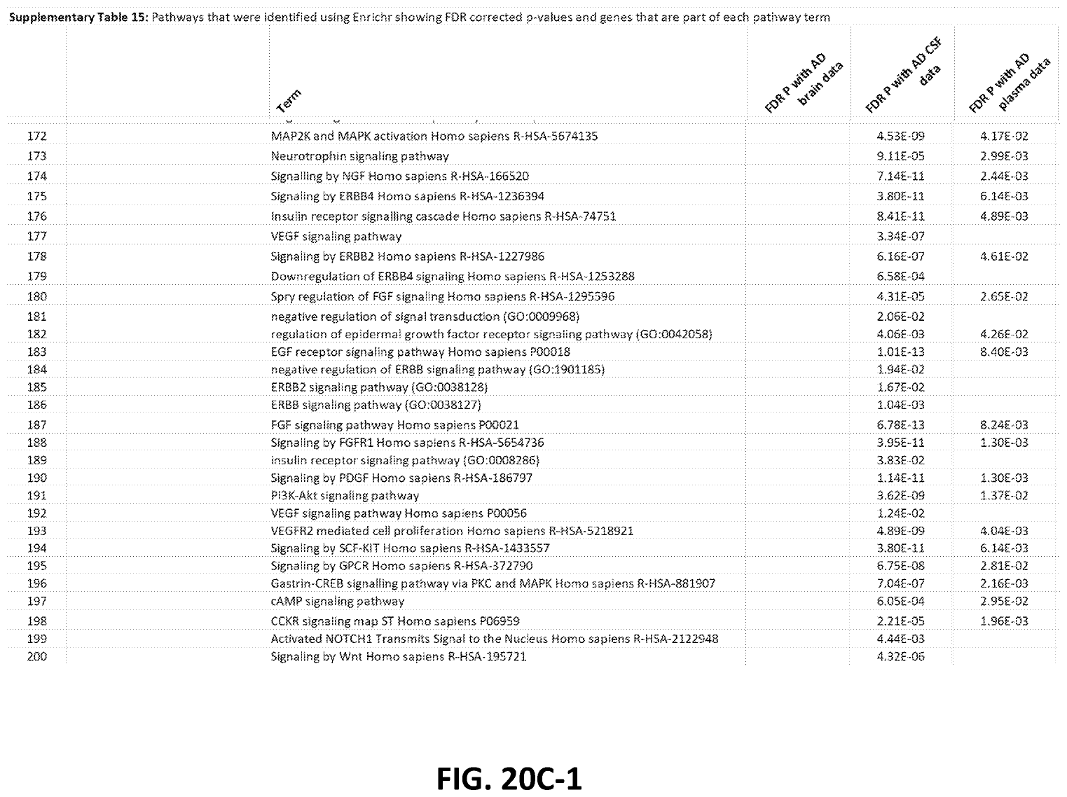

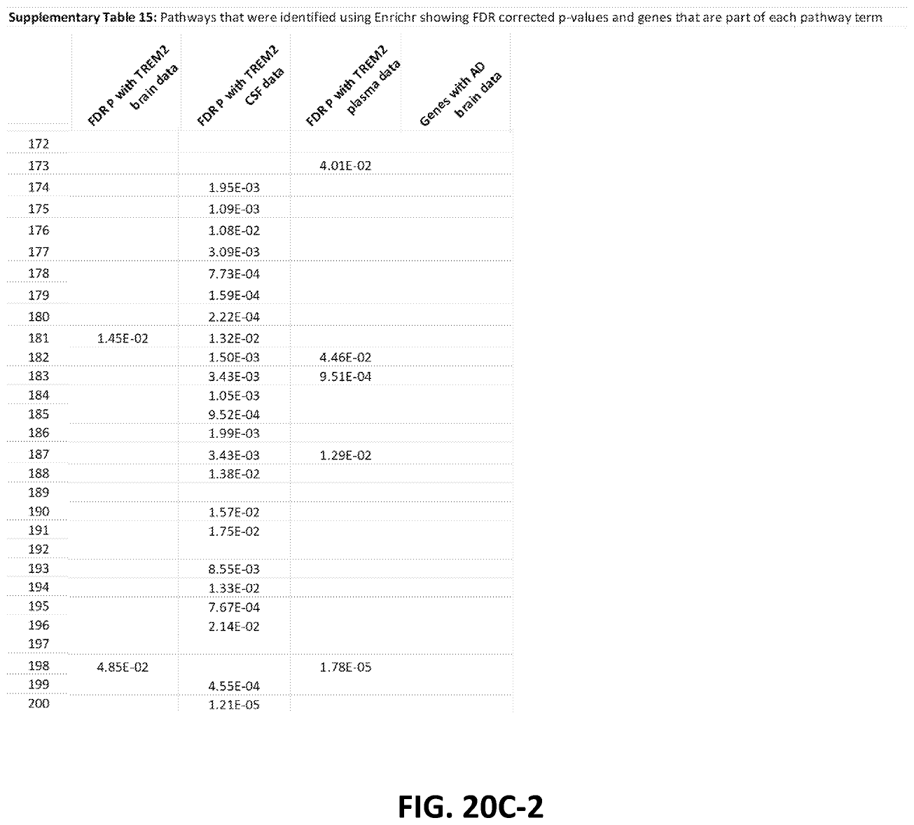

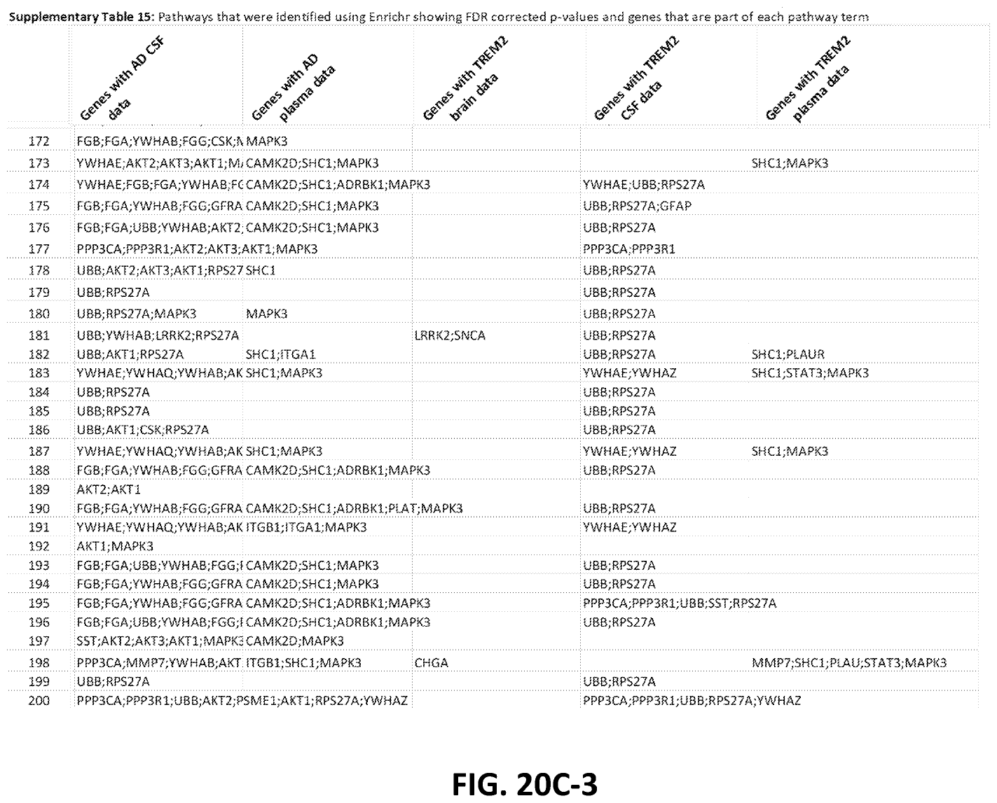

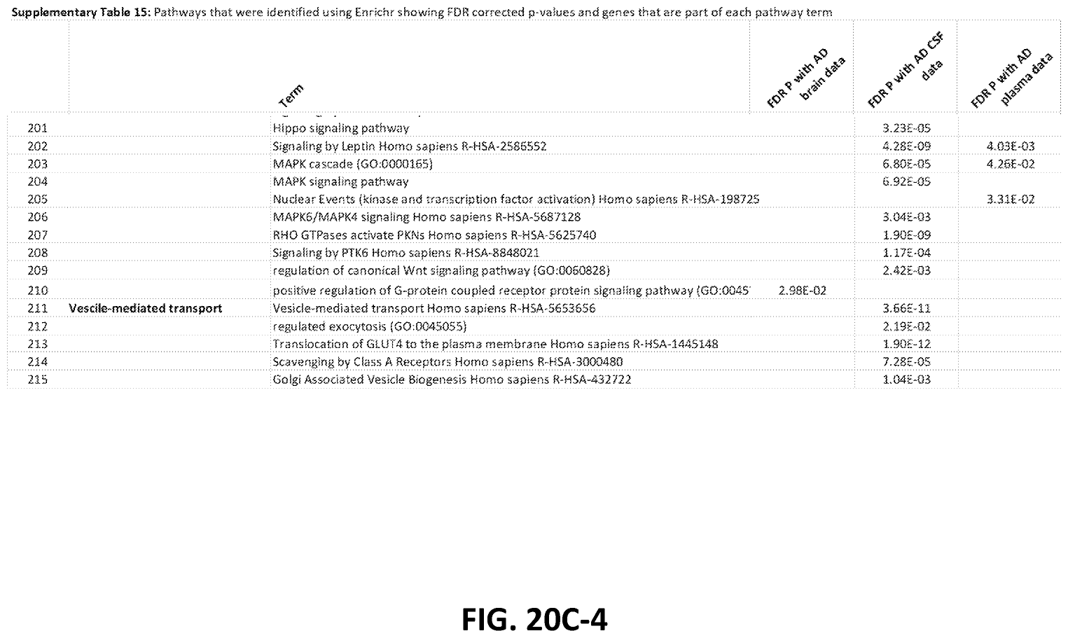

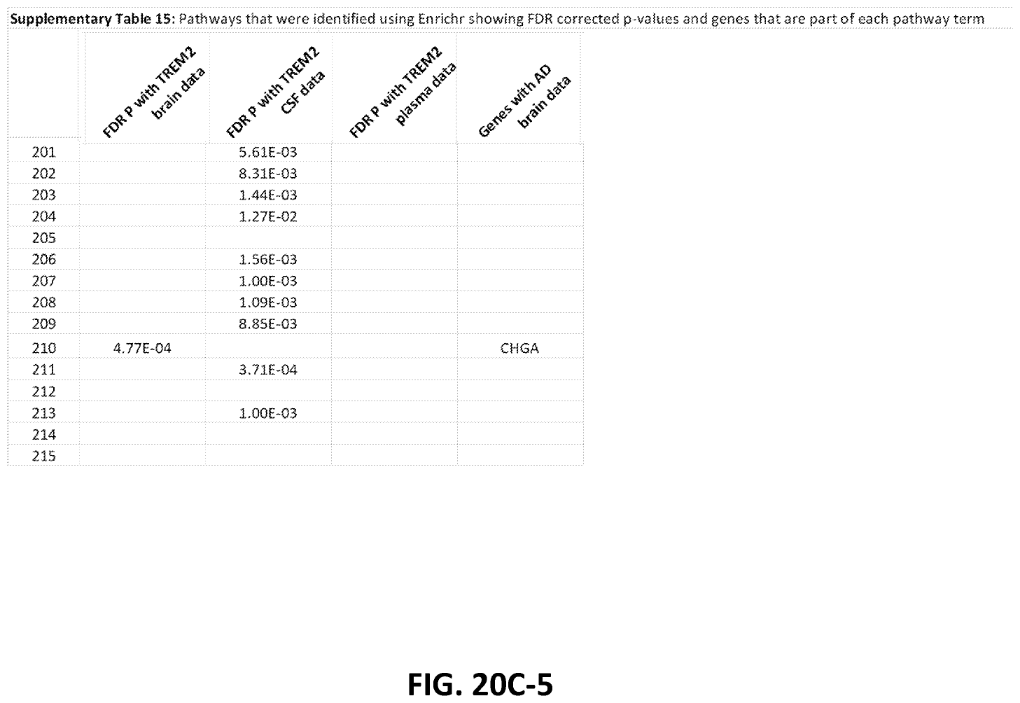

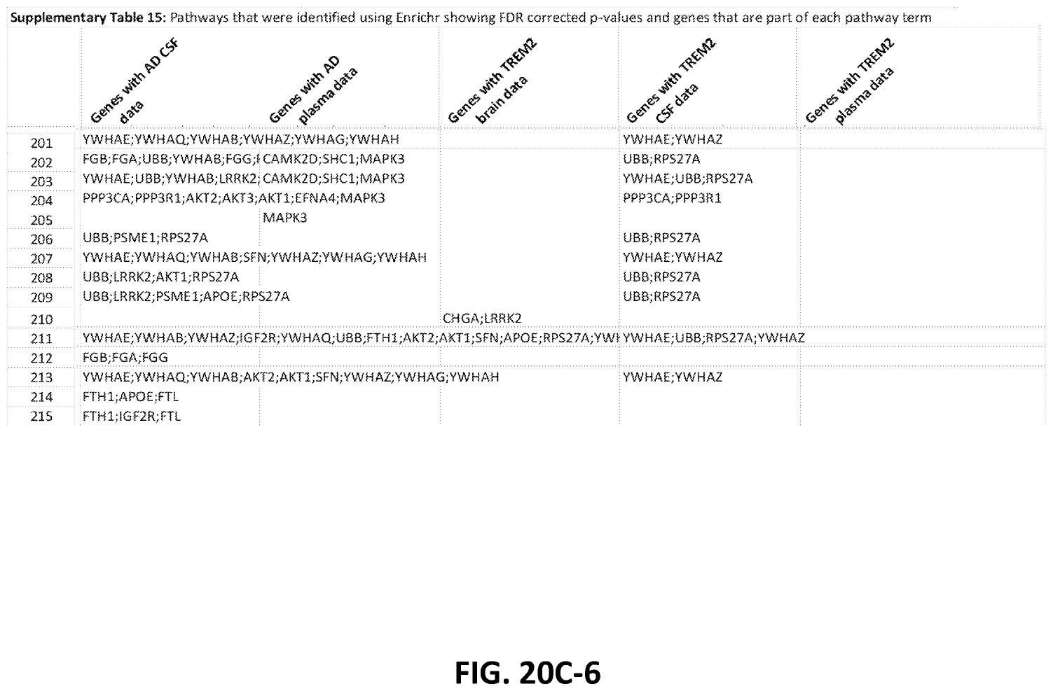

Finally, the proteins identified in the analyses were determined to be enriched in common functional pathways. Functional enrichment analysis was performed with Enrichr. As expected, the AD pathway was significant in CSF in both the sporadic AD (FDR=1.9×10⁻³) and TREM2 variant-specific analyses (FDR=5.8×10⁻³, Supplementary Table 15 in FIG. 20A (20A-1 through 20A-9), 20B (20B-1 through 20B-9), and 20C (20C-1 through 20C-6)). The proteins that are part of this pathway that were identified in the analyses include APOE, calcineurin (PPP3R1 and PPP3CA), and MAPK3 (FIG. 5(A-B)). APOE is the strongest and most common genetic risk factor for AD, and individuals with the APOE ε4 allele have lower CSF Aβ42 levels and lower Aβ42 clearance. Genetic variants in calcineurin have been associated with higher CSF p-tau levels and earlier age at onset. MAPK3 has also been reported to be involved in AD pathology, likely by affecting tau phosphorylation. In any biomarker discovery study, it is often difficult to determine whether the proteins identified are part of a causal pathway or just a product of the disease. Several facts strongly suggest that many of the proteins identified in this study are, in fact, causal. As mentioned, APOE is known to be part of the causal AD pathway, and calcineurin and MAPK3 have recently been reported as part of the causal AD pathway by pQTL and Mendelian randomization analyses.

Several proteins that are part of the Parkinson disease pathway, including α-synuclein, LRRK2, granulin, and UCHL1, were also found to be dysregulated in CSF and plasma for the sporadic AD and TREM2 analyses (FDR<3.4×10⁻³, Supplementary Table 15 in FIG. 20A (20A-1 through 20A-9), 20B (20B-1 through 20B-9), and 20C (20C-1 through 20C-6)). On autopsy, around 30% of the AD cases, including autosomal dominant AD, present with Lewy bodies, which are deposits of α-synuclein. Those reports, together with these analyses, indicate that PD pathology shares similarities with AD pathology. Similar to α-synuclein, LRRK2 also showed a strong association with autosomal dominant AD (P=7.7×10⁻⁴) and TREM2 (P=9.3×10⁻⁶). The GRN gene, which encodes the granulin protein, was initially associated with frontotemporal dementia, but recent, large GWAS have also found GRN in both AD and PD.

Granulin, implicated in wound healing as a part of the innate immune response pathway, was also found to be enriched in the proteomic analyses for sporadic AD in CSF (FDR=6.9×10⁻⁹) and plasma (FDR=2.1×10⁻³), as well as the CSF TREM2-specific analyses (FDR=1.1×10⁻³). Other dysregulated proteins identified in the analyses that are also part of this pathway include SHC1, MAPK3, ITGB1, and SPP1, among others. SPP1 has recently been implicated in microglia activation and the AD pathway. Similar to SPP1, ITBG1 is a microglia gene and has been shown to be differentially expressed in the hippocampus and peripheral blood mononuclear cells (PBMC) of AD cases, important in microglia activation, and part of the causal pathway in network analyses. Recent studies have also demonstrated that meningeal lymphatics affect microglia and AD risk. The analyses also found several endothelial-specific proteins (ERK-1, SHC1, and BCAM).

The 17 proteins that were associated with ADAD status in both brain and CSF in the same direction, were also enriched for proteins part of the Alzheimer's disease pathway (p<1×10⁻⁴) and the cellular response to chemical stimulus pathway (go:0.0070887; p=0.034), which includes, among others, MIF, a pro-inflammatory cytokine involved on involved in the innate immune response; LILRB; and CD22 also part of the immune response pathway. IDE is involved in the cellular breakdown of insulin and has been reported to be involved in the degradation and clearance of naturally secreted amyloid beta-protein by neurons and microglia.

In summary, the proteins dysregulated in the analyses are not randomly distributed across functional groups; they are enriched in specific pathways known to be implicated in AD and other pathways (PD, immune response) that may be instrumental to AD pathophysiology and may represent new therapeutic targets. Indeed, the analyses indicate that the proteins identified herein are not only dysregulated in AD but also play a causal role.

Discussion

This is the first large-scale, multi-tissue proteomic characterization of sporadic and genetically defined AD cases (TREM2 and Mendelian cases). A web portal was created to facilitate the exploration of the analyses and further investigation into individual protein abundance levels across disease status or sex. In this study, proteomic measures were obtained from Knight ADRC and DIAN cohorts and proteomic profiles were identified for sporadic AD, TREM2 variant carriers, and autosomal dominant AD cases in three tissues. These proteomic profiles replicated in independent datasets and across tissues, which were used to create tissue-specific prediction models and to identify novel causal proteins and pathways for sporadic and genetically defined AD cases. Tissue-specific prediction models were created and validated using proteins identified in CSF and plasma that were as good as, or better than, the current gold standard antibody-based biomarkers for AD risk. Having new prediction models in CSF and plasma that are independent from Aβ and tau may be relevant for clinical trials and therapies that target those molecules, as biomarkers that do not rely on the target protein may be needed. It was also demonstrated that there are common proteins associated with AD status across tissues, which has important implications for the identification and validation of AD biomarkers in future studies.

This study also identified new proteins and pathways implicated in sporadic AD and individuals with specific genetic profiles. These results highlight the need for multi-tissue proteomics to fully understand the biology of AD and create tissue-specific prediction models for individuals with specific genetic profiles, ultimately supporting its utility in generating clinically useful biomarker arrays. This study indicates that once individuals with specific genetic profiles are identified, it is possible to create customized prediction models and identify proteins implicated in disease, an instrumental step toward creating individualized, specific disease risk evaluation and treatment.

Methods and Materials

Study Participants

This study included the brain (N=360), CSF (N=717), and plasma (N=490) data from the Knight ADRC and the Dominantly Inherited Alzheimer Network (DIAN) cohorts. The recruited individuals were evaluated by Clinical Core personnel of the Knight ADRC. For brain samples, brain autopsy was performed by the Knight ADRC Neuropathology Core and AD status was determined by postmortem neuropathological analysis. Brain tissues were collected from fresh frozen human parietal lobes. Neuropathological phenotypes, including Braak tau, CERAD Aβ, α-synuclein pathology, postmortem interval (PMI), age at onset, age at death and brain weight, were obtained for all brain samples. The brain data included 24 individuals carrying autosomal dominant AD (ADAD) mutations, out of which 18 were from the DIAN cohort. Among these ADAD individuals, 19, 1, and 4 carried pathogenic mutations in PSEN1, PSEN2, and APP, respectively.

Among individuals with CSF and plasma data, AD cases corresponded to those with a diagnosis of dementia of the Alzheimer's type (DAT) using criteria equivalent to the National Institute of Neurological and Communication Disorders and Stroke-Alzheimer's Disease and Related Disorders Association for probable AD, and AD severity was determined using the Clinical Dementia Rating (CDR®) at the time of lumbar puncture (for CSF samples) or blood draw (for plasma samples). Controls received the same assessment as the cases but were non-demented (CDR=0). CSF and blood for plasma were collected in the morning after an overnight fast, aliquoted, and stored at −80° C. until assayed. CSF Aβ and tau levels were measured as published previously. The Institutional Review Board of Washington University School of Medicine in St. Louis approved the study and research was performed in accordance with the approved protocols.

Proteomic Data

For deep omics characterization in brain, CSF, and plasma tissues, the level of 1,305 proteins was quantified using a multiplexed, single-stranded DNA aptamer assay developed by SomaLogic. The assay covers a dynamic range of 108 and measures all three major categories: secreted, membrane, and intracellular proteins. The proteins cover a wide range of molecular functions and include proteins known to be relevant to human disease. Aliquots of gray matter homogenate (150 μl) of tissue were provided to the Genome Technology Access Center at Washington University in St. Louis for protein measurement. As previously published, 17 modified single-stranded DNA aptamers are used to bind specific protein targets, which are then quantified by a DNA microarray. Protein concentrations are quantified as relative fluorescent units (RFU) of intensity in this DNA microarray.

Quality control (QC) was performed at the sample and aptamer levels using control aptamers (positive and negative controls) and calibrator samples. At the sample level, hybridization controls on each plate were used to correct for systematic variability in hybridization. The median signal over all aptamers was used to correct for within-run technical variability. This median signal was assigned to different dilution sets within each tissue. For brain and CSF samples, a 20% dilution rate was used. For plasma samples, three different dilution sets (40%, 1%, and 0.005%) were used.

As described previously, additional QC was performed by identifying and removing protein/analyte outliers by applying the following four criteria using R. 1) Minimum detection filtering. The limit of detection (LOD) was computed based on negative controls. If the average expression of an analyte in a sample was found to be less than its LOD in more than 15% of total sample size, this sample was marked as an outlier and excluded. 2) Scale factor difference. Scale factor difference was calculated as the maximum value of the absolute difference between the median expression of analytes per plate and calibration scale factor. If the maximum difference was greater than 0.5, the analyte was excluded. 3) Coefficient of variation (CV). The CV for each aptamer was calculated as the standard deviation divided by the mean of the protein levels in calibrators. If the median coefficient of variation for a particular analyte was greater than 0.15, this analyte was excluded. 4) Interquartile range (IQR). If more than 15% of the log 10 transformed analyte values are located outside of either end of a 1.5-fold of IQR, this analyte was marked as an outlier and excluded. In addition, if more than 15% of the transformed analyte values in a particular sample are located outside a 1.5-fold of IQR, this sample was marked as an outlier and excluded. Analytes and samples that remained after applying these 4 criteria were used for the downstream statistical analysis.

Differential Abundance Analysis

To obtain proteomic signatures of sporadic AD status, TREM2 risk variant carriers, and autosomal dominant AD (ADAD) status, differential abundance analysis was performed by using log₁₀-transformed protein levels as an outcome in a linear regression model. In all three tissues, sporadic AD status and TREM2 variant carrier status were considered as a main predictor. In brain tissue, ADAD status was also considered. In each tissue, surrogate variable analysis (SVA) was performed while including status and age as covariates in a null hypothesis model to remove batch effects in the proteomics data (17 batches in brain, 50 batches in CSF and 27 batches in plasma data) and correct for other unmeasured heterogeneity. The number of resulting surrogate variables were 10, 32, and 14 in brain, CSF, and plasma, respectively. Age at death or at measurement (in all regression models except for ADAD-specific analysis), sex and the resulting surrogate variables were included as covariates. In ADAD analysis in brain tissue, sex was excluded from covariates as control group was older than ADAD individuals.

In addition, analyses were performed using age-at-onset (AAO) and AD neuropathology characteristics (Braak neurofibrillary tangle scores and CDR at death) for brain data, AAO and CSF pTau/Aβ42 ratio for CSF data, and AAO for plasma data, while including the same covariates. For AAO, survival analysis was performed while considering age, sex, and surrogate variables as covariates. A survival object was created using R function Surv and a Cox proportional hazards regression model was performed using the coxph function. In addition, the consistency between effect sizes of AD status and AD neuropathology measures was examined through the scatter plots. Correlation tests were performed using cor.test in R to test association between effect sizes with Pearson's product moment correlation coefficient and two-sided alternative hypothesis. In addition, Fisher's exact test was performed for the same direction.

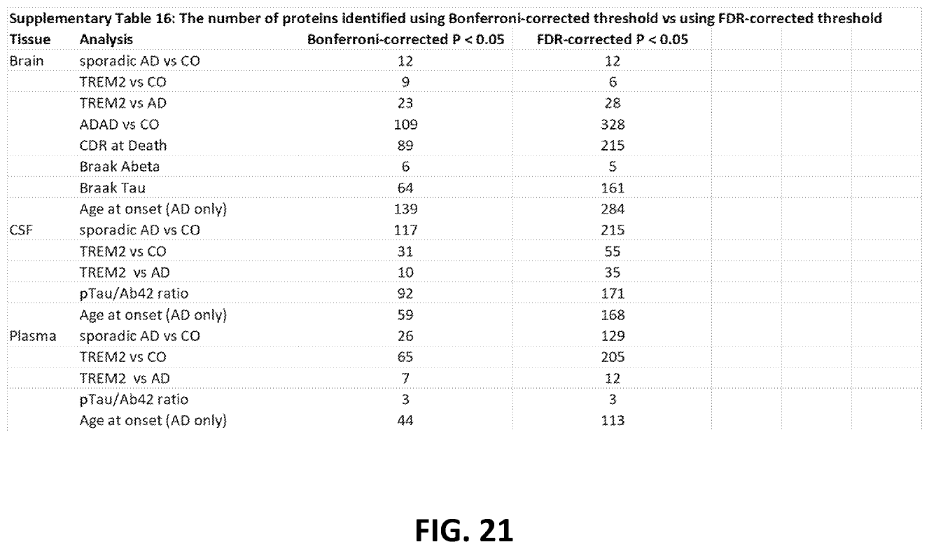

The minimum number of principal components (PCs) was obtained that cumulatively explain 95% of the variance for each tissue after QC. The number of PCs is 75, 169, and 230 in brain, CSF, and plasma data, respectively. A Bonferroni-corrected threshold was considered as 0.05 divided by this number of PCs. The thresholds corresponded to 0.67×10⁻⁴ in brain, 2.96×10⁻⁴ in CSF, and 2.21×10⁴ in plasma. When Bonferroni correction and false discovery rate (FDR) was applied, the use of these Bonferroni-corrected thresholds usually provided fewer significant results and is therefore more conservative than the use of FDR (Supplementary Table 16 in FIG. 21). Because of this, Bonferroni correction was applied.

Replication Strategies

To internally validate the identified proteins in each tissue, which proteins would be associated in the remaining tissues at the nominal significance threshold (P<0.05) were examined. In addition, to externally replicate the sporadic AD findings within the same tissue, multiple publicly available proteomic datasets were downloaded.

For brain tissue, the mass-spectrometry data that were processed and deposited by Johnson et al were downloaded for the following 6 studies: the Adult Changes In Thought (ACT), Banner Sun Health Research Institute (BANNER), Baltimore longitudinal study of aging (BLSA), Mayo Clinic (MAYO), Mount Sinai Brain Bank (MSBB), the Religious Orders Study and the Memory and Aging Project (ROSMAP). These brain proteomics data were combined from all 6 studies (resulting in a total of 10,078 proteins measured in 415 AD patients and 194 controls) and SVA was performed to account for batch effects and unmeasured heterogeneity. Then differential abundance levels of AD status were performed jointly while considering age, sex and 11 surrogate variables as covariates. In addition, to confirm that the results were not false positives due to the joint analysis merging of all 6 studies, the results presented by Johnson et al. that used ACT, BANNER, BLSA, MSBB, results by Higginbotham et al. that used DLPFC, and results by Wingo et al. that used ROSMAP were used.

For CSF tissue, multiple reaction monitoring ADNI data were obtained and differential analysis performed for 320 proteins in 188 AD patients and 75 controls while considering age, sex and 7 surrogate variables as covariates. In addition, the results based on Emory-ADRC mass-spectrometry data from Higginbotham et al. and the results based on BioFinder OLINK data from Whelan et al. were used. For plasma tissue, the cleaned SOMAscan 1.1K proteomic data from the AddNeuroMed study was downloaded. After excluding 166 individuals with mild cognitive impairment, differential abundance analysis of 320 AD patients and 194 controls was performed, while including sex, age, batch effects, and APOE status as covariates. For ADAD status, only high throughput proteomic screening was performed on brain tissue. To replicate these proteins that were associated with ADAD status in brain, analysis in CSF from 289 ADAD mutation carriers and 184 non carriers from the DIAN study was performed, while including sex, age, and batch effects as covariates.

To compute the fold-enrichment of replication to what would be expected by chance, the Binomial distribution was used. Under the null hypothesis of no enrichment, the expected number of replicated proteins is the number of available proteins for testing times the significance threshold (0.05×0.05 in across-tissue replication and 0.05×0.5 in external replication). The enrichment was computed as the ratio of the observed replications by the expected replications and the p-value based on the Binomial probability.

ADNI

Data used in the preparation of this article were obtained from the Alzheimer's Disease Neuroimaging Initiative (ADNI) database. The primary goal of ADNI has been to test whether serial magnetic resonance imaging (MRI), positron emission tomography (PET), other biological markers, and clinical and neuropsychological assessment can be combined to measure the progression of mild cognitive impairment (MCI) and early Alzheimer's disease (AD).

Prediction Models

To obtain tissue-specific prediction models, logistic regression models considering multiple proteins as main predictors and sporadic AD and TREM2 variant carrier status as an outcome were performed, while including sex, age, and with and without APOE ε4 allele status as covariates. In sporadic AD status, externally validated proteins were used for both discovery and replication datasets. In brain, the combined mass-spectrometry brain proteomics data from ACT, BANNER, BLSA, MSBB, MAYO, and ROSMAP was used for replication. In CSF, the Emory-ADRC mass-spectrometry data that were processed and deposited by Higginbotham et al. were downloaded and used for replication. In plasma, cleaned data from the AddNeuroMed SOMAscan 1.1K proteomic dataset were used for replication. In TREM2 variant carrier status modeling, proteins validated across tissues were used for discovery data.

When there were more than 10 proteins, stepwise regression analysis was also performed to reduce the number of proteins by selecting the best model by Akaike information criterion (AIC) using step function in R. Both forward and backward selection were considered and the model with fewer proteins was chosen when there were two competing models. The well accepted CSF biomarker using p-Tau/Aβ42 ratio was considered as a gold standard for comparison. Receiver operator characteristic (ROC) curves and areas under the curves (AUC) were computed using the R package pROC V1.12.1. The roc.test function within the same package was used to compare AUC values for two models (such as AUC based on the identified proteins vs. AUC based on p-Tau/Aβ42 ratio).

Pathway Enrichments

Functional enrichment analysis was performed with Enrichr.26. For sporadic AD findings, the genes that target the identified proteins and validated internally or externally (9, 42 and 14 genes in brain, CSF, and plasma, respectively) were used as an input for enrichment analysis. For TREM2 carrier status, 6, 10 and 21 genes were considered that replicated across tissues, in brain, CSF, and plasma, respectively. Among multiple gene-set libraries, KEGG, Reactome, Panther pathways and GO biological process were considered. The significance of functional enrichment was reported as the p-value of Fisher's exact test, followed by Benjamini-Hochberg adjustment for false discovery rates (FDR) in testing multiple hypotheses. Results with FDR<0.05 were considered as significant and included for creating the dot chart and tile plots to graphically display the findings.

What is claimed is:

1. A method of diagnosing or prognosing Alzheimer's disease (AD), AD severity, or AD response or monitoring disease progression or treatment in a subject comprising:
obtaining or having obtained a biological sample; and
measuring levels of proteins (see e.g., Supplementary Tables 1-16 in FIGS. 6-21).

2. The method of claim 1, wherein the proteins are associated with TREM2 risk variant status, sporadic AD, AD risk, or AD onset, AD status, age of AD onset, or ADAD mutation carrier status.

3. The method of claim 1, wherein the biological sample is cerebrospinal fluid (CSF), plasma, or brain tissue.

4. The method of claim 3, wherein the biological sample is CSF, plasma, or brain tissue and one or more of the following proteins are measured or detected:

| | | | | | ADAD |
|---|---|---|---|---|---|
| Sporadic AD vs | | | TREM2 variant | | vs CO |
| controls | | | carriers | | Brain |
| Brain | CSF | Plasma | CSF | Plasma | & CSF |
| 8 | 12 | 9 | 7 | 9 | 17 |
| Midkine | 14-3-3 | ERK-1 | 14-3-3E | Bone | FSTL1 |
| | protein | | | proteo- | |
| | zeta/delta | | | glycan | |
| | | | | II | |
| SMOC1 | EphA5 | BARK1 | 14-3-3 | STAT3 | SMOC1 |
| | | | protein | | |
| | | | zeta/delta | | |
| CgA | Calcineurin | GNS | Somato- | uPA | Angio- |
| | | | statin-28 | | poietin-4 |
| HGF | Somato- | CAMK2D | SMOC1 | ERK-1 | HGF |
| | statin-28 | | | | |
| NRX1B | Cyclophilin | CDON | Ubiquitin + | VCAM-1 | UBC9 |
| | A | | 1 | | |
| UBC9 | Contactin-5 | HMG-1 | QORL1 | PAPP-A | CD22 |
| NET1 | GFAP | tPA | Calcineurin | BSSP4 | AMPM2 |
| SAP | Cortico- | RELT | | XTP3A | IDE |
| | tropin- | | | | |
| | lipotropin | | | | |
| | Spondin-1 | Integrin | | S100A4 | SARP-2 |
| | | a1b1 | | | |
| | TCTP | | | | XPNPEP1 |
| | Poly- | | | | Peroxi- |
| | Ubiquitin | | | | redoxin-6 |
| | K48 | | | | |
| | Peroxi- | | | | ILT-2 |
| | redoxin-6 | | | | |
| | | | | | MIF |
| | | | | | Poly- |
| | | | | | Ubiquitin |
| | | | | | K48 |
| | | | | | NET1 |
| | | | | | CSRP3 |
| | | | | | ATPO |

5. The method of claim 1, wherein if the protein level is elevated compared to expected or control value the subject is at risk for AD, sporadic AD, TREM2 variant, or ADAD variant.

6. The method of claim 4, wherein elevation of at least one protein selected from the set of 9 proteins in plasma predict sporadic disease.

7. The method of claim 4, wherein elevation of at least one protein selected from the set of 12 proteins in CSF predicts sporadic disease status.

8. The method of claim 4, wherein at least one protein selected from the set of 9 proteins in plasma predicts TREM2 risk variant carrier status.

9. The method of claim 4, wherein elevation of at least one protein selected from the set of 7 proteins in CSF predicts TREM2 risk variant carrier status.

10. The method of claim 4, wherein elevation of at least one protein selected from the set of 17 proteins in brain tissue and plasma predicts autosomal dominant AD.

11. The method of claim 1, wherein detection of elevated levels of proteins indicates the subject is at risk for or has sporadic AD and/or AD-risk variants in TREM2 or pathogenic variants in APP and PSEN1/2.

12. The method of claim 1, wherein a subject has or is suspected of having AD, sporadic AD and/or AD-risk variants in TREM2 or pathogenic variants in APP and PSEN1/2.

13. The method of claim 1, wherein the protein levels are being monitored before, during, and/or after treatment.

14. The method of claim 1, wherein the protein levels are being monitored as a marker of disease progression.

15. The method of claim 4, wherein a proteomic signature for TREM2 variant carriers differentiate TREM2 variant carriers from sporadic AD cases.
